(19) United States
(12) Patent Application Publication
(10) Pub. No.: US 2008/0132525 A1
(43) Pub. Date: Jun. 5, 2008

(54) Inhibitors of DNA Methyltransferase

(75) Inventors: Amal Wahhab, Laval (CA); Jeffrey M. Besterman, Baie D'Urfe (CA); Daniel Delorme, St-Lazare (CA); Isakovic Ljubomir, Montreal (CA); David Llewellyn, Vancouver (CA); Jubrail Rahil, Montreal (CA); Oscar Saavedra, Outremont (CA); Robert Deziel, Mount-Royal (CA)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: METHYLGENE INC., St-Laurent (CA)

(21) Appl. No.: 11/566,441

(22) Filed: Dec. 4, 2006

(51) Int. Cl. A61K 31/52; A61K 31/44; A61P 35/00; C07D 471/02; C07D 473/00

(52) U.S. Cl. 514/263.4; 435/184; 514/300; 544/277; 546/113

(57) ABSTRACT

The invention relates to the inhibition of DNA methyltransferase isoforms DNMT1 and DNMT3b2. The invention provides compounds and methods for inhibiting DNMT1 and DNMT3b2.

### BACKGROUND OF THE INVENTION

1. Field of the Invention

This invention relates to inhibition of the DNA methyltransferase isoforms DNMT1 and DNMT3b2. More particularly, the invention relates to compounds and methods for the inhibition of DNMT1 and DNMT3b2.

2. Summary of the Related Art

Changes in human DNA methylation patterns are commonly found in human tumors and are implicated in development and maintenance of human cancer (A. Bird, Genes Dev., 2002, 16, 6-21; P. Siedlecki, R. G. Boy, S. Comagic, R. Schirrmacher, M. Wiessler, P. Zielenkiewicz, S. Suhai, and F. Lyko, Biochemical and Biophysical Research communications, 2003, 306, 558). DNA hypermethylation in cancer cells results in alteration of gene expression patterns and most notably the loss of expression of tumor suppressor genes (K. D. Robertson, A. P. Baylin, Nat. Rev. Genet., 2000, 1, 11-19; P. A. Jones, S. B. Baylin, Trends Genet., 2000, 16, 168-174).

DNA methyltransferase 1 (DNMT1) protein is a major contributor to DNA methyltransferase activity in human cells and is required to maintain methylation patterns in differentiated cells (M. F. Robert, S. Morin, N. Beaulieu, F. Gauthier, I. C. Chute, A. Barsalou, A. R. MacLeod, Nat. Genet. 33 (2003) 61-65). The de novo DNA methyltransferases DNMT3A and DNMT3B establish DNA methylation during early embryogenesis (M. Okano, S. Xie, E. Li, Nat. Genet., 1998, 19, 219-220; M. Okano, D. W. Bell, D. A. Haber, E. Li, Cell, 1999, 99, 247-257, T. M. Geiman, U. T. Sankpal, A. K. Robertson, Y. Chen, M. Mazumdar, J. T. Heale, J. A. Schmeising, W. Kim, K. Yokomori, Y. Zhao, and K. D. Robertson, Nucleic Acids Research, 2004, 32, 2716-2729).

Regulation of methylation of human DNA requires the activity of several DNA methyltransferases. The DNA is covalently modified by the cofactor (L)-S-Adenosyl-L-methionine (SAM) at carbon-5 of cytosine residues. This biological methylating agent is converted to its demethylated metabolite (L)-S-Adenosyl-L-homocysteine (L-SAH). In tissue the SAM and SAH levels are equivalent (F. Salvatore, R. Utili, V. Zappia, and S. K. Shapiro, Anal. Biochem. 1971, 41, 16; J. Hoffman, Anal. Biochem, 1975, 68, 522). SAH can bind to DNA methyltransferases and inhibit their catalytic reaction and is an important molecule in the regulation of biological transmethylation (T. Deguchi and J. Barchas, J. Biol. Chem., 1971, 246, 3175; A. Oliva, P. Galletti, and V. Zappia, Eurp. J. Biochem., 1980, 104, 595; P.M. Ueland and J. Saebo, Biochemistry, 1979, 18, 4130).

Inhibitors of DNMT1 and DNMT3B are expected to have value as anti-cancer agents (M. Szyf, Fontiers in Bioscience 2001, 6, 599-609; M. F. Robert, S. Morin, N. Beaulieu, F. Gauthier, I. C. Chute, A. Barsalou, A. R. MacLeod, Nat. Genet. 2003, 33, 61-65; D. S. Schrump et al, abstract 442, 16^{th} EORTC-AACR Symposium on Molecular Targets and Cancer Therapeutics, 28 Sep.-1 Oct. 2004, Geneva, Switzerland).

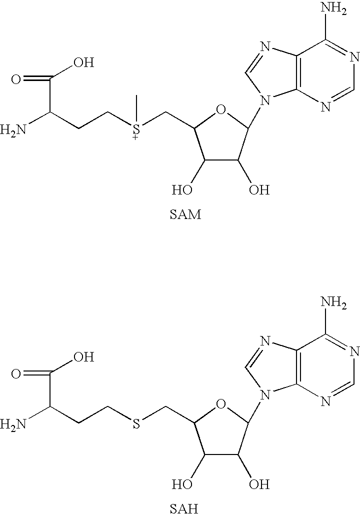

Borchardt et al. described the synthesis and biological activity of a number of SAH analogues having structural modifications in the amino acid, sugar, or base portions. However, these analogues were synthesized in an effort to develop inhibitors for O— or N-methyltransferases, specifically catechol O-methyltransferase (COMT), hydroxyindole O-methyltransferase (HIMT), indoleethylamine N-methyltransferase (INMT), phenylethanolamine N-methyltransferase (PNMT), and histamine N-methyltransferase (HMT), but not human DNA methyltransferase (DNMT) (R. T. Borchardt and Y. S. Wu, J. Med. Chem., 1974, 17, 862; R. T. Borchardt, J. A. Huber, and Y. S. Wu, J. Med. Chem., 1974, 17, 868; R. T. Borchardt and Y. S. Wu, J. Med. Chem., 1975, 18, 300; R. T. Borchardt and Y. S. Wu, J. Med. Chem., 1976, 19, 197; R. T. Borchardt, Biochem. Pharmacol., 1975, 24, 1542; R. T. Borchardt, J. A. Huber, and Y. S. Wu, J. Med. Chem., 1976, 19, 1094). Some N-6-substituted SAH analogues have been tested against protein arginine methyltransferases (Q. Lin et al, J. Am. Chem. Soc. 2001, 123, 11608-11613).

SAH is a known nonselective inhibitor of human DNA methyltransferase and many other methyltransferases. A number of simplified and less rigid analogues of SAH have been reported. Such compounds, however, were devoid of activity against human DNA methyltransferase (M. Botta, R. Saladino, G. Pedraly-Noy, and R. Nicoletti, Med. Chem. Res., 1994, 4, 323).

The 2′- and 3′-hydroxy groups of the sugar portion and the 6-amino group of the adenine moiety of SAH were found to be important structural requirements for inhibition of DNA methyltransferases. T. Borchardt, Y. S. Wu, and J. A. Huber, J. Med. Chem., 1976, 19, 1104; M. D. Houston, B. Matuszewska, and R. T. Borchardt, J. Med. Chem. 1985, 28, 478; P. A. Crooks, M. J. tribe, and R. J. Pinney, J. Pharm. Pharmacol, 1984, 36, 85.

SAM and SAH as inhibitors (in vivo) are not good drugs and they are unstable in plasma due to hydrolases and ribonucleases, they have poor absorption due to the Zwitterionic nature, are rapidly excreted, and have a short half life. A. A. Minnick and G. L. Kenyon, J. Org. Chem., 1988, 53, 4952. In addition, SAH is an endogenous inhibitor of numerous methyltransferases and as such is non-selective, making it undesirable as a drug.

Some more stable nitrogen anlogues of SAM and SAH have been reported. Chi-Deu Chang and J. K. Coward, J. Med. Chem., 1976, 19, 684; A. A. Minnick and G. Kenyon, J. Org. Chem., 1988, 53, 4952; M. Thompson, a. Makhalfia, D. P. Hornby, and G. M. Blackburn, J. Org. Chem. 1999, 64, 7467 When these analogues were evaluated as inhibitors of catechol O-methyltransferase and tRNA methylases, however, they were found to have poor activity.

Sinefungin, a natural product, is a nitrogen analogue of SAH and has been reported to inhibit human DNA methyltransferase. It is also a non-selective inhibitor with potential for toxicity (C. Barbes, J. Sanchez, M. J. Yebra, M. Robert-Gero, and C. Hardisson, FEMS Microbiology Letters, 1990, 69, 239).

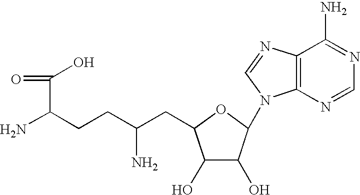

In addition, inhibition of DNMT1 with 5-azacytidine or related compounds has been reported. Such inhibitors, however, are incorporated into the DNA of the target cell and, thus, suffer from high toxicity and low specificity (D. V. Santi, A. Norment, C. E. Garrett, Proc. Natl. Acad. Sci. USA 81(1984) 6993-6997).

More recently indolyl-2-phenyl bisamidines have been reported as a new class of DNA methyltransferase inhibitors. These are known to be DNA binding agents, however, and thus may inhibit the DNMTs indirectly. S. W. Goldstein, B. L. Mylari, J. R. Perez, and E. A. Glazer, U.S. Pat. No. 6,699,862 B1, Mar. 2, 2004.

This invention provides compounds and methods for the inhibition of human C-5 (cytosine) DNA methyltransferases DNMT1 and DNMT3b2 enzymes.

### BRIEF SUMMARY OF THE INVENTION

The compounds of the invention are inhibitors of the DNA methyltransferase isoforms DNMT1 and DNMT3b2. Accordingly, the invention provides new inhibitors of DNMT1 and DNMT3b2.

In a first aspect, the invention provides compounds of formula (I) (and their pharmaceutically acceptable salts) that are useful as inhibitors of DNMT1 and/or DNMT3b2 and therefore are useful for studying the role of DNA methyltransferase in biological processes.

In a second aspect, the invention provides compositions comprising a compound that is an inhibitor of DNMT1 and/or DNMT3b2, or a pharmaceutically acceptable salt thereof, and a pharmaceutically acceptable carrier, excipient, or diluent.

In a third aspect, the invention provides a method of inhibiting DNMT1 and/or DNMT3b2 enzymes in a cell, comprising contacting a cell in which inhibition of DNMT1 or DNMT3b2 is desired with a compound of the invention.

The foregoing merely summarizes certain aspects of the invention and is not intended to be limiting in nature. These aspects and other aspects and embodiments are described more fully below.

### BRIEF DESCRIPTION OF THE DRAWINGS

FIG. 1 displays the full length DNMT1 (Swissprot accession number P26358 (SEQ ID NO.1)).

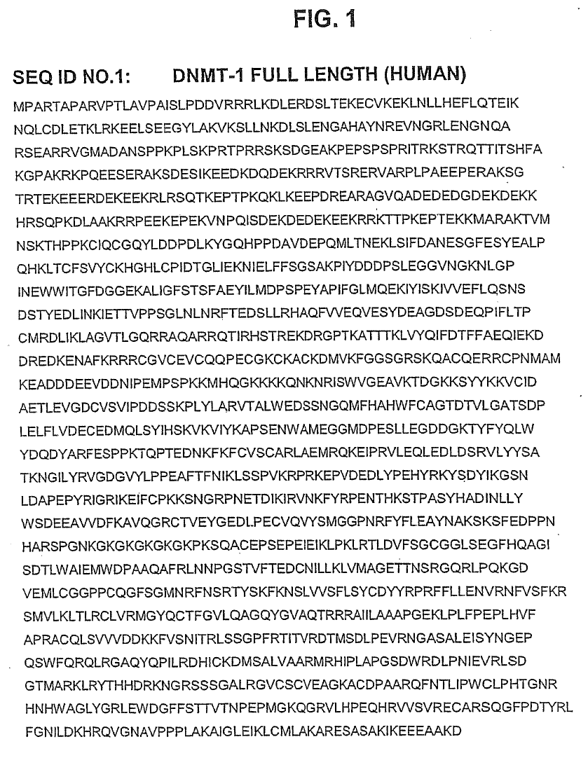

FIG. 2 displays the DNMT3 splice variant 2 of DNMT3b2 (Swissprot accession number Q9UBC3-2 (SEQ ID NO.2))

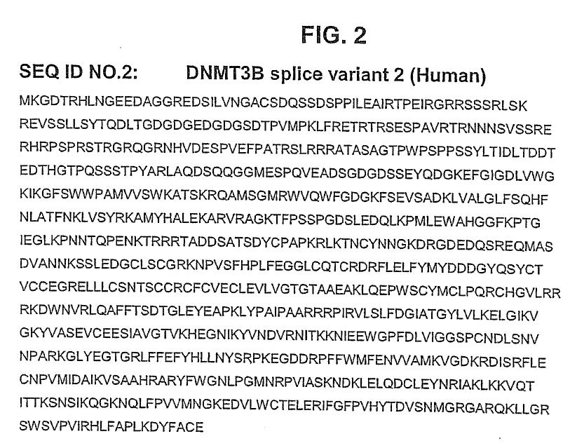

### DETAILED DESCRIPTION OF THE PREFERRED EMBODIMENTS

The invention provides compounds and methods for inhibiting DNMT1 and DNMT3b2.

The patent and scientific literature referred to herein establishes knowledge that is available to those with skill in the art. The issued patents, applications, and references that are cited herein are hereby incorporated by reference to the same extent as if each was specifically and individually indicated to be incorporated by reference. In the case of inconsistencies, the present disclosure will prevail.

For purposes of the present invention, the following definitions will be used (unless expressly stated otherwise):

The term “inhibitor of DNMT1 and DNMT3b2” is used to identify a compound having a structure as defined herein, which is capable of interacting with DNMT1, DNMT3b2 or both DNMT1 and DNMT3b2 and inhibiting the activity of DNMT1, DNMT3b2, or both DNMT1 and DNMT3b2. In some preferred embodiments, such reduction of activity is at least about 50%, more preferably at least about 75%, and still more preferably at least about 90%.

For simplicity, chemical moieties are defined and referred to throughout primarily as univalent chemical moieties (e.g., alkyl, aryl, etc.). Nevertheless, such terms are also used to convey corresponding multivalent moieties under the appropriate structural circumstances clear to those skilled in the art. For example, while an “alkyl” moiety generally refers to a monovalent radical (e.g. CH₃—CH₂—), in certain circumstances a bivalent linking moiety can be “alkyl,” in which case those skilled in the art will understand the alkyl to be a divalent radical (e.g., —CH₂—CH₂—), which is equivalent to the term “alkylene.” (Similarly, in circumstances in which a divalent moiety is required and is stated as being “aryl,” those skilled in the art will understand that the term “aryl” refers to the corresponding divalent moiety, arylene.) All atoms are understood to have their normal number of valences for bond formation (i.e., 4 for carbon, 3 for N, 2 for O, and 2, 4, or 6 for S, depending on the oxidation state of the S). On occasion a moiety may be defined, for example, as (A)ₐ-B—, wherein a is 0 or 1. In such instances, when a is 0 the moiety is B— and when a is 1 the moiety is A-B—. Also, a number of moieties disclosed herein exist in multiple tautomeric forms, all of which are intended to be encompassed by any given tautomeric structure.

The term “hydrocarbyl” refers to a straight, branched, or cyclic alkyl, alkenyl, or alkynyl, each as defined herein. A “C₀” hydrocarbyl is used to refer to a covalent bond. Thus, “C₀-C₃-hydrocarbyl” includes a covalent bond, methyl, ethyl, ethenyl, ethynyl, propyl, propenyl, propynyl, and cyclopropyl.

The term “alkyl” as employed herein refers to straight and branched chain aliphatic groups having from 1 to 12 carbon atoms, preferably 1-8 carbon atoms, and more preferably 1-6 carbon atoms, which is optionally substituted with one, two or three substituents. Preferred alkyl groups include, without limitation, methyl, ethyl, propyl, isopropyl, butyl, isobutyl, sec-butyl, tert-butyl, pentyl, and hexyl. A “C₀” alkyl (as in “C₀-C₃₋alkyl”) is a covalent bond (like “C₀” hydrocarbyl).

The term “alkenyl” as used herein means an unsaturated straight or branched chain aliphatic group with one or more carbon-carbon double bonds, having from 2 to 12 carbon atoms, preferably 2-8 carbon atoms, and more preferably 2-6 carbon atoms, which is optionally substituted with one, two or three substituents. Preferred alkenyl groups include, without limitation, ethenyl, propenyl, butenyl, pentenyl, and hexenyl.

The term “alkynyl” as used herein means an unsaturated straight or branched chain aliphatic group with one or more carbon-carbon triple bonds, having from 2 to 12 carbon atoms, preferably 2-8 carbon atoms, and more preferably 2-6 carbon atoms, which is optionally substituted with one, two or three substituents. Preferred alkynyl groups include, without limitation, ethynyl, propynyl, butynyl, pentynyl, and hexynyl.

An “alkylene,” “alkenylene,” or “alkynylene” group is an alkyl, alkenyl, or alkynyl group, as defined hereinabove, that is positioned between and serves to connect two other chemical groups. Preferred alkylene groups include, without limitation, methylene, ethylene, propylene, and butylene. Preferred alkenylene groups include, without limitation, ethenylene, propenylene, and butenylene. Preferred alkynylene groups include, without limitation, ethynylene, propynylene, and butynylene.

The term “cycloalkyl” as employed herein includes saturated and partially unsaturated cyclic hydrocarbon groups having 3 to 12 carbons, preferably 3 to 8 carbons, and more preferably 3 to 6 carbons, wherein the cycloalkyl group additionally is optionally substituted. Preferred cycloalkyl groups include, without limitation, cyclopropyl, cyclobutyl, cyclopentyl, cyclopentenyl, cyclohexyl, cyclohexenyl, cycloheptyl, and cyclooctyl.

The term “heteroalkyl” refers to an alkyl group, as defined hereinabove, wherein one or more carbon atoms in the chain are replaced by a heteroatom selected from the group consisting of O, S, NH, N-alkyl, SO, SO₂, SO₂NH, or NHSO₂.

An “aryl” group is a C₆-C₁₄ aromatic moiety comprising one to three aromatic rings, which is optionally substituted. Preferably, the aryl group is a C₆-C₁₀ aryl group. Preferred aryl groups include, without limitation, phenyl, naphthyl, anthracenyl, and fluorenyl. An “aralkyl” or “arylalkyl” group comprises an aryl group covalently linked to an alkyl group, either of which may independently be optionally substituted or unsubstituted. Preferably, the aralkyl group is (C₁-C₆)alk(C₆-C₁₀)aryl, including, without limitation, benzyl, phenethyl, and naphthylmethyl.

A “heterocyclyl” or “heterocyclic” group is a ring structure having from about 3 to about 12 atoms, wherein one or more atoms are selected from the group consisting of N, O, S, SO, and SO₂. The heterocyclic group is optionally substituted on carbon at one or more positions. The heterocyclic group is also independently optionally substituted on nitrogen with alkyl, aryl, aralkyl, alkylcarbonyl, alkylsulfonyl, arylcarbonyl, arylsulfonyl, alkoxycarbonyl, or aralkoxycarbonyl. Preferred heterocyclic groups include, without limitation, epoxy, aziridinyl, tetrahydrofuranyl, pyrrolidinyl, piperidinyl, piperazinyl, thiazolidinyl, oxazolidinyl, oxazolidinonyl, and morpholino. In certain preferred embodiments, the heterocyclic group is fused to an aryl, heteroaryl, or cycloalkyl group. Examples of such fused heterocyles include, without limitation, tetrahydroquinoline and dihydrobenzofuran. Specifically excluded from the scope of this term are compounds where an annular O or S atom is adjacent to another O or S atom.

As used herein, the term “heteroaryl” refers to groups having 5 to 14 ring atoms, preferably 5, 6, 9, or 10 ring atoms; having 6, 10, or 14 n-electrons shared in a cyclic array; and having, in addition to carbon atoms, from one to three heteroatoms per ring selected from the group consisting of N, O, and S. The term “heteroaryl” is also meant to encompass monocyclic and bicyclic groups. For example, a heteroaryl group may be pyrimidinyl, pyridinyl, benzimidazolyl, thienyl, benzothiazolyl, benzofuranyl and indolinyl. A “heteroaralkyl” or “heteroarylalkyl” group comprises a heteroaryl group covalently linked to an alkyl group, either of which is independently optionally substituted or unsubstituted. Preferred heteroalkyl groups comprise a C₁-C₆ alkyl group and a heteroaryl group having 5, 6, 9, or 10 ring atoms. Specifically excluded from the scope of this term are compounds having adjacent annular O and/or S atoms. Examples of preferred heteroaralkyl groups include pyridylmethyl, pyridylethyl, pyrrolylmethyl, pyrrolylethyl, imidazolylmethyl, imidazolylethyl, thiazolylmethyl, and thiazolylethyl. Specifically excluded from the scope of this term are compounds having adjacent annular O and/or S atoms.

For simplicity, reference to a “Cₙ-Cₘ” heterocyclyl or “Cₙ-Cₘ” heteroaryl means a heterocyclyl or heteroaryl having from “n” to “m” annular atoms, where “n” and “m” are integers. Thus, for example, a C₅-C₆-heterocyclyl is a 5- or 6-membered ring having at least one heteroatom, and includes pyrrolidinyl (C₅) and piperidinyl (C₆); C₆-hetoaryl includes, for example, pyridyl and pyrimidyl.

An “arylene,” “heteroarylene,” or “heterocyclylene” group is an aryl, heteroaryl, or heterocyclyl group, as defined hereinabove, that is positioned between and serves to connect two other chemical groups.

Preferred heterocyclyls and heteroaryls include, but are not limited to, acridinyl, azocinyl, benzimidazolyl, benzofuranyl, benzothiophenyl, benzoxazolyl, benzthiazolyl, benztriazolyl, pyridotriazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,3,4-triazolyl, and xanthenyl.

As employed herein, when a moiety (e.g., cycloalkyl, hydrocarbyl, aryl, heteroaryl, heterocyclic, urea, etc.) is described as “optionally substituted” it is meant that the group optionally has from one to four, preferably from one to three, more preferably one or two, non-hydrogen substituents. Suitable substituents include, without limitation, halo, hydroxy, oxo (e.g., an annular —CH— substituted with oxo is —C(O)—) nitro, halohydrocarbyl, hydrocarbyl, aryl, aralkyl, alkoxy, aryloxy, amino, acylamino, alkylcarbamoyl, arylcarbamoyl, aminoalkyl, acyl, carboxy, hydroxyalkyl, alkanesulfonyl, arenesulfonyl, alkanesulfonamido, arenesulfonamido, aralkylsulfonamido, alkylcarbonyl, acyloxy, cyano, and ureido groups. Preferred substituents, which are themselves not further substituted (unless expressly stated otherwise) are:
(a) halo, hydroxy, cyano, oxo, carboxy, formyl, nitro, amino, amidino, guanidino,
(b) C₁-C₅ alkyl or alkenyl or arylalkyl imino, carbamoyl, azido, carboxamido, mercapto, hydroxy, hydroxyalkyl, alkylaryl, arylalkyl, C₁-C₈ alkyl, C₁-C₈ alkenyl, C₁-C₈ alkoxy, C₁-C₈ alkoxycarbonyl, aryloxycarbonyl, C₂-C₈ acyl, C₂-C₈ acylamino, C₁-C₈ alkylthio, arylalkylthio, arylthio, C₁-C₈ alkylsulfinyl, arylalkylsulfinyl, arylsulfinyl, C₁-C₈ alkylsulfonyl, arylalkylsulfonyl, arylsulfonyl, C₀-C₆ N.alkyl carbamoyl, C₂-C₁₅ N,N-dialkylcarbamoyl, C₃-C₇ cycloalkyl, aroyl, aryloxy, arylalkyl ether, aryl, aryl fused to a cycloalkyl or heterocycle or another aryl ring, C₃-C₇ heterocycle, C₅-C₁₄ heteroaryl, or any of these rings fused or spiro-fused to a cycloalkyl, heterocyclyl, or aryl, wherein each of the foregoing is further optionally substituted with one more moieties listed in (a), above; and
(c) —(CH₂)ₛ—NR³⁰R³¹, wherein s is from 0 (in which case the nitrogen is directly bonded to the moiety that is substituted) to 6, and R³⁰ and R³¹ are each independently hydrogen, cyano, oxo, carboxamido, amidino, C₁-C₈ hydroxyalkyl, C₁-C₃ alkylaryl, aryl-C₁-C₃ alkyl, C₁-C₈ alkyl, C₁-C₈ alkenyl, C₁-C₈ alkoxy, C₁-C₈ alkoxycarbonyl, aryloxycarbonyl, aryl-C₁-C₃ alkoxycarbonyl, C₂-C₈ acyl, C₁-C₈ alkylsulfonyl, arylalkylsulfonyl, arylsulfonyl, aroyl, aryl, cycloalkyl, heterocyclyl, or heteroaryl, wherein each of the foregoing is further optionally substituted with one more moieties listed in (a), above; or
   R³⁰ and R³¹ taken together with the N to which they are attached form a heterocyclyl or heteroaryl, each of which is optionally substituted with from 1 to 3 substituents from (a), above.

A “halohydrocarbyl” is a hydrocarbyl moiety in which from one to all hydrogens have been replaced with one or more halo.

The term “halogen” or “halo” as employed herein refers to chlorine, bromine, fluorine, or iodine. As herein employed, the term “acyl” refers to an alkylcarbonyl or arylcarbonyl substituent. The term “acylamino” refers to an amide group attached at the nitrogen atom (i.e., R—CO—NH—). The term “carbamoyl” refers to an amide group attached at the carbonyl carbon atom (i.e., NH₂—CO—). The nitrogen atom of an acylamino or carbamoyl substituent is additionally substituted. The term “sulfonamido” refers to a sulfonamide substituent attached by either the sulfur or the nitrogen atom. The term “amino” is meant to include NH₂, alkylamino, arylamino, and cyclic amino groups. The term “ureido” as employed herein refers to a substituted or unsubstituted urea moiety.

The term “radical” as used herein means a chemical moiety comprising one or more unpaired electrons.

A moiety that is substituted is one in which one or more hydrogens have been independently replaced with another chemical substituent. As a non-limiting example, substituted phenyls include 2-flurophenyl, 3,4-dichlorophenyl, 3-chloro-4-fluoro-phenyl, 2-fluoro-3-propylphenyl. As another non-limiting example, substituted n-octyls include 2,4-dimethyl-5-ethyl-octyl and 3-cyclopentyl-octyl. Included within this definition are methylenes (—CH₂—) substituted with oxygen to form carbonyl —CO—).

An “unsubstituted” moiety as defined above (e.g., unsubstituted cycloalkyl, unsubstituted heteroaryl, etc.) means that moiety as defined above that does not have any of the optional substituents for which the definition of the moiety (above) otherwise provides. Thus, for example, while an “aryl” includes phenyl and phenyl substituted with a halo, “unsubstituted aryl” does not include phenyl substituted with a halo.

Throughout the specification, preferred embodiments of one or more chemical substituents are identified. Also preferred are combinations of preferred embodiments. For example, paragraph [0052] describes preferred embodiments of L₁ in the compounds of formula (II-A) and paragraph [0053] describes preferred embodiments of R₂ in the compounds of formula (II-A). Thus, also contemplated as within the scope of the invention are compounds of formula (A) in which L₁ and R₂ are as described in paragraph [0052] and R₁ is as described in paragraph [0053]. Furthermore, compounds excluded from any one particular genus of compounds (e.g., through a proviso clause) are intended to be excluded from the scope of the invention entirely, including from other disclosed genera, unless expressly stated to the contrary.

### Compounds

In the first aspect, the invention comprises compounds of formula (I), that are inhibitors DNMT1 and DNMT3b2:

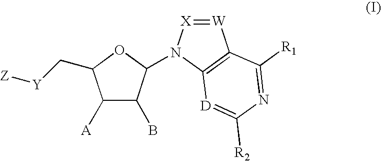

and pharmaceutically acceptable salts and complexes thereof, wherein
R₁ is H or NR₃P₄;
   R₃ and R₄ independently represent H, C₁-C₆ alkyl, C₂-C₆ alkenyl, C₂-C₆ alkynyl, cycloalkyl, heterocyclyl, aryl, heteroaryl, —C₁-C₆-alkyl-cycloalkyl, —C₁-C₆-alkyl-heterocyclyl, —C₁-C₆-alkyl-aryl, —C₁-C₆-alkyl-heteroaryl, —C₁-C₆alkoxy-aryl or —(CH₂)₁₋₆-T, wherein C₁-C₆ alkyl, C₂-C₆ alkenyl, C₂-C₆ alkynyl, cycloalkyl, heterocyclyl, aryl and heteroaryl, at each occurrence, are optionally substituted; or
   R₃ and R₄ taken together with the nitrogen to which they are attached form a C₅-C₉ heterocyclyl ring or a heteroaryl ring, wherein said ring is optionally substituted;
   T is NH—C(═O)—R₁₄, —NH—SO₂—R₁₅, or —S—(CH₂)₁₋₃—R₁₄,
   R₁₄ is C₁-C₆ alkyl, aryl or heteroaryl and R₁₅ is aryl, wherein C₁-C₆ alkyl, aryl, and heteroaryl, at each occurrence, are optionally substituted;
R₂ is H, halo, CF₃, C₁-C₆ alkyl, C₂-C₆ alkenyl, C₂-C₆ alkynyl, C₁-C₆ alkoxy, —NH—C₁-C₆ alkyl, or —S—C₁-C₆ alkyl, wherein C₁-C₆ alkyl, C₂-C₆ alkenyl, and C₂-C₆ alkynyl are each optionally substituted;
A and B independently are F, Cl, —OH, H, —NHR, or —OR;
   R at each occurrence is independently benzyl or C₁-C₄ alkyl, wherein benzyl and C₁-C₄ alkyl are optionally substituted;
W is CH, N, CR, or C-halogen;
X is CH, N, C—C₁-C₆-alkyl, or C-halogen;
D is CH, or N;
Y is —S—, —O—, N(R₁₆)—, —CH═CH—, —S—CH₂—, —O—CH₂—,
   where R₁₆ is H, C₁-C₆ alkyl, —C₁-C₆-alkyl-aryl, —C₁-C₆-alkyl-heteroaryl, or —C₂-C₆ alkenyl-aryl, wherein C₁-C₆ alkyl, aryl, and heteroaryl, at each occurrence, are optionally substituted;
Z is -L-C(H)(NH₂)—COOR₇, -L-NR₁₉R₂₀, or heterocyclyl, wherein heterocyclyl is optionally substituted;
   L is a bond or is —(CR₁₇R₁₈)₁₋₆;
      each R₁₇ and R₁₈ independently is H or C₁-C₆-alkyl, wherein C₁-C₆-alkyl is optionally substituted;
   R₁₉ and R₂₀ independently are H, C₁-C₆-alkyl, heteroaryl, or H₂N—C(═NH)—CH₂—, wherein C₁-C₆-alkyl and heteroaryl are optionally substituted; and
   R₇ is H or C₁-C₆-alkyl.

In a preferred embodiment of the compounds according to paragraph [0047], the compounds are represented by formula (II)

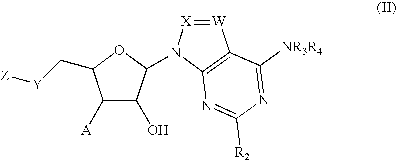

and pharmaceutically acceptable salts and complexes thereof, wherein
A is H, halogen, or OH;
R₂ is H, halo, C₁-C₆ alkyl, C₂-C₆ alkenyl or —S—C₁-C₆ alkyl, wherein C₁-C₆ alkyl and C₂-C₆ alkenyl, at each occurrence, are optionally substituted;
R₃ and R₄ independently represent H, C₁-C₆ alkyl, C₂-C₆ alkenyl, cycloalkyl, heterocyclyl, aryl, heteroaryl, —C₁-C₆-alkyl-cycloalkyl, —C₁-C₆-alkyl-heterocyclyl, —C₁-C₆-alkyl-aryl, —C₁-C₆-alkyl-heteroaryl, —C₁-C₆alkoxy-aryl or —(CH₂)₁₋₆-T, wherein C₁-C₆ alkyl, C₂-C₆ alkenyl, cycloalkyl, heterocyclyl, aryl and heteroaryl, at each occurrence, are optionally substituted; or
R₃ and R₄ taken together with the nitrogen to which they are attached form a C₅-C₉ heterocyclyl ring or a heteroaryl ring, wherein said ring is optionally substituted;
   T is NH—C(═O)—R₁₄, —NH—SO₂—R₁₅, or —S—(CH₂)₁₋₃—R₁₄,
   R₁₄ is C₁-C₆ alkyl, aryl or heteroaryl and R₁₅ is aryl, wherein C₁-C₆ alkyl, aryl, and heteroaryl, at each occurrence, are optionally substituted;
W and X are independently CH or N;
Y is S, O, or N(R₁₆)—,
   R₁₆ is H, C₁-C₆ alkyl, —C₁-C₆-alkyl-aryl, —C₁-C₆-alkyl-heteroaryl, or —C₂-C₆ alkenyl-aryl, wherein C₁-C₆ alkyl, aryl, and heteroaryl, at each occurrence, are optionally substituted;
Z is -L-C(H)(NH₂)—COOR₇, -L-NR₁₉R₂₀, or heterocyclyl, wherein heterocyclyl is optionally substituted;
   L is a bond or is —(CR₁₇R₁₈)₁₋₆—;
      R₁₇ and R₁₈ independently are H or C₁-C₆-alkyl, wherein C₁-C₆-alkyl is optionally substituted;
   R₁₉ and R₂₀ independently are H, C₁-C₅-alkyl, heteroaryl, or H₂N—C(═NH)—CH₂—, wherein C₁-C₆-alkyl and heteroaryl are optionally substituted; and
   R₇ is H or C₁-C₆-alkyl.

In a preferred embodiment of the compounds according to paragraph [0048], the compounds are of formula II-A:

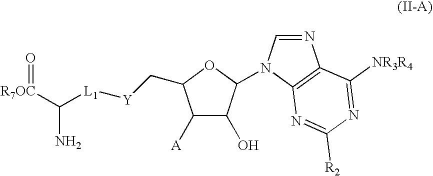

and pharmaceutically acceptable salts and complexes thereof, wherein
A is H, halogen, or OH;
R₂ is H, halo, C₁-C₆ alkyl, C₂-C₆ alkenyl or —S—C₁-C₆ alkyl, wherein C₁-C₆ alkyl and C₂-C₆ alkenyl, at each occurrence, are optionally substituted;
R₃ and R₄ independently represent H, C₁-C₆ alkyl, C₂-C₆ alkenyl, cycloalkyl, heterocyclyl, aryl, heteroaryl, —C₁-C₆-alkyl-cycloalkyl, —C₁-C₆-alkyl-heterocyclyl, —C₁-C₆-alkyl-aryl, —C₁-C₆-alkyl-heteroaryl, —C₁-C₆alkoxy-aryl or —(CH₂)₁₋₆-T, wherein C₁-C₆ alkyl, C₂-C₆ alkenyl, cycloalkyl, heterocyclyl, aryl and heteroaryl, at each occurrence, are optionally substituted; or
R₃ and R₄ taken together with the nitrogen to which they are attached form a C₅-C₉ heterocyclyl ring or a heteroaryl ring, wherein said ring is optionally substituted;
   T is NH—C(═O)—R₁₄, —NH—SO₂—R₁₅, or —S—(CH₂)₁₋₃—R₁₄, R₁₄ is C₁-C₆ alkyl, aryl or heteroaryl and R₁₅ is aryl, wherein C₁-C₆ alkyl, aryl, and heteroaryl, at each occurrence, are optionally substituted;
R₇ is H or C₁-C₆-alkyl;
Y is S, O, or —N(R₁₆)—,
   R₁ is H, C₁-C₆ alkyl, —C₁-C₆-alkyl-aryl, —C₁-C₆-alkyl-heteroaryl, or —C₂-C₆ alkenyl-aryl, wherein C₁-C₆ alkyl, aryl, and heteroaryl, at each occurrence, are optionally substituted; and
L₁ is —(CR₁₇R₁₈)₁₋₆—; and
   R₁₇ and R₁₈ independently are H or C₁-C₆-alkyl, wherein C₁-C₆-alkyl is optionally substituted.

In a preferred embodiment of the compounds according to paragraph [0049], A is OH.

In a preferred embodiment of the compounds according to paragraph [0049]-[00500, R₇ is H.

In a preferred embodiment of the compounds according to paragraphs [0049]-[0051], L₁ is —CH₂CH₂—.

In a preferred embodiment of the compounds according to paragraphs [0049]-[0052], R₂ is H, halogen, C₁-C₃ alkyl, —S—C₁-C₂ alkyl, or C₂-C₃ alkenyl.

In a preferred embodiment of the compounds according to paragraphs [0049]-[0053], R₂ is H or halogen.

In a preferred embodiment of the compounds according to paragraphs [0049]-[0054], Y is S.

In a preferred embodiment of the compounds according to paragraphs [0049]-[0055], R₃ and R₄ are independently H, C₁-C₆ alkyl, C₂-C₆ alkenyl, cycloalkyl, heterocyclyl, aryl, heteroaryl, —C₁-C₆-alkyl-cycloalkyl, —C₁-C₆-alkyl-aryl, —C₁-C₆-alkyl-heteroaryl, —C₁-C₆alkoxy-aryl or —(CH₂)₁₋₆-T, wherein C₁-C₆ alkyl, C₂-C₆ alkenyl, cycloalkyl, heterocyclyl, aryl and heteroaryl, at each occurrence, are optionally substituted, or R₃ and R₄ taken together with the nitrogen to which they are attached form a C₅-C₉ heterocyclyl ring or a heteroaryl ring, wherein said ring is optionally substituted.

In a preferred embodiment of the compounds according to paragraph [0056], R₃ and R₄ are both H.

In a preferred embodiment of the compounds according to paragraph [0056], R₃ is H and R₄ is C₁-C₆ alkyl optionally substituted with 1, 2, or 3 groups independently selected from OH, CO₂H, NH₂, N(C₁-C₃ alkyl)₂, C₁-C₃ alkoxy, and phenyl.

In a preferred embodiment of the compounds according to paragraph [0056], both R₃ and R₄ are C₁-C₆ alkyl, wherein C₁-C₆ alkyl is independently optionally substituted with 1, 2, or 3 groups independently selected from OH, CO₂H, NH₂, N(C₁-C₃ alkyl)₂, C₁-C₃ alkoxy, and phenyl.

In a preferred embodiment of the compounds according to paragraph [0056], R₃ is —C₁-C₃-alkyl-aryl and R₄ is C₁-C₆ alkyl, wherein C₁-C₆ alkyl is optionally substituted with 1, 2, or 3 groups independently selected from OH, CO₂H, NH₂, N(C₁-C₃ alkyl)₂, C₁-C₃ alkoxy, and phenyl.

In a preferred embodiment of the compounds according to paragraph [0056], R₃ is H and R₄ is C₃-C₈-cycloalkyl. Preferably, cycloalkyl is cyclopropyl, cyclohexyl, or cyclooctanyl.

In a preferred embodiment of the compounds according to paragraph [0056], R₃ is H and R₄ is aryl, wherein aryl is optionally substituted. Preferably aryl is phenyl, naphthyl, or fluorenyl. Preferably, aryl is unsubstituted or is substituted with 1, 2, or 3 groups independently selected from OCH₃, NH₂, NHCH₃, NO₂, C₁-C₃ alkyl, halogen, CF₃, CN, OH, NH₂SO₂—, and phenyl.

In a preferred embodiment of the compounds according to paragraph [0056], R₃ is H and R₄ is heterocyclyl. Preferably, heterocyclyl is pyrrolidinonyl.

In a preferred embodiment of the compounds according to paragraph [0056], R₃ is H and R₄ is —C₁-C₆-alkyl-heteroaryl, wherein heteroaryl and C₁-C₆ alkyl are optionally substituted. Preferably, heteroaryl is imidazolyl, indolyl, thiophenyl, pyridinyl, or dihydroindenyl. Preferably, heteroaryl is unsubstituted or is substituted with 1, 2, or 3 groups independently selected from methoxy and phenyl-C₁-C₃-alkoxy-.

In a preferred embodiment of the compounds according to paragraph [0056], R₃ is H and R₄ is —C₁-C₆-alkyl-aryl, wherein aryl and C₁-C₆ alkyl are optionally substituted. Preferably, aryl is phenyl, naphthyl, or fluorenyl. Preferably, aryl is unsubstituted or is substituted with 1, 2, or 3 groups independently selected from OCH₃, NH₂, NHCH₃, NO₂, C₁-C₃ alkyl, halogen, CF₃, CN, OH, NH₂SO₂—, or phenyl.

In a preferred embodiment of the compounds according to paragraph [0065], aryl is phenyl, optionally substituted with 1, 2, or 3 groups independently selected from OCH₃, NH₂, NHCH₃, NO₂, C₁-C₃ alkyl, halogen, CF₃, CN, OH, NH₂SO₂—, or phenyl.

In a preferred embodiment of the compounds according to paragraph [0056], R₃ is H and R₄ is —(CH₂)₁₋₆-T, wherein T is NH—C(═O)—R₁₄, —NH—SO₂—R₁₅, or —S—(CH₂)₁₋₃-R₁₄, R₁₄ is C₁-C₆ alkyl, aryl or heteroaryl and R₁₅ is aryl, wherein C₁-C₆ alkyl, aryl, and heteroaryl, at each occurrence, are optionally substituted.

In a preferred embodiment of the compounds according to paragraph [0067], T is selected from —NH—CO-phenyl, NH—SO₂-naphthyl, —S—CH₂-phenyl, —NH—CO-methyl, and —NH—CO-furanyl.

In a preferred embodiment of the compounds according to paragraph [0056], R₃ and R₄ taken together with the nitrogen to which they are attached form a C₅-C₉ heterocyclyl ring or a heteroaryl ring, wherein said ring is optionally substituted.

In a preferred embodiment of the compounds according to paragraph [0069], R₃ and R₄ taken together with the nitrogen to which they are attached form a C₄-C₈ heterocyclyl ring, wherein said ring is optionally substituted.

In a preferred embodiment of the compounds according to paragraph [0069]-[0070], R₃ and R₄ taken together with the nitrogen to which they are attached form a pyrrolidinyl, azetidinyl, or piperidinyl ring.

In a preferred embodiment of the compounds according to paragraphs [0049]-[0054], Y is —N(R₁₆)—.

In a preferred embodiment of the compounds according to paragraph [0072], R₁₆ is H.

In a preferred embodiment of the compounds according to paragraph [0072], R₁₆ is C₁-C₆-alkyl. Preferably, C₁-C₆-alkyl is unsubstituted or is substituted with NO₂.

In a preferred embodiment of the compounds according to paragraph [0072], R₁₆ is aryl, wherein aryl is optionally substituted.

In a preferred embodiment of the compounds according to paragraph [0072], R₁₆ is —C₁-C₆-alkyl-aryl, wherein C₁-C₆-alkyl and aryl are optionally substituted.

In a preferred embodiment of the compounds according to paragraph [0072], R₁₆ is —C₂-C₆ alkenyl-aryl, wherein aryl is optionally substituted.

In a preferred embodiment of the compounds according to paragraphs [0075]-[0077], aryl is phenyl, wherein phenyl is unsubstituted or is substituted with 1, 2, or 3 groups independently selected from NO₂, C₁-C₃-alkoxy, CN, or CF₃.

In a preferred embodiment of the compounds according to paragraph [0072], R₁₆ is —C₁-C₆-alkyl-heteroaryl, wherein C₁-C₆-alkyl and heteroaryl are optionally substituted.

In a preferred embodiment of the compounds according to paragraph [0079], heteroaryl is pyridinyl.

In a preferred embodiment of the compounds according to paragraphs [0072]-[0080], R₃ and R₄ are both H.

In a preferred embodiment of the compounds according to paragraphs [0072]-[0080], R₃ is H, and R₄ is C₁-C₆-alkyl.

In a preferred embodiment of the compounds according to paragraphs [0049]-[0054], Y is oxygen.

In a preferred embodiment of the compounds according to paragraph [0048], the compounds are of formula II-B:

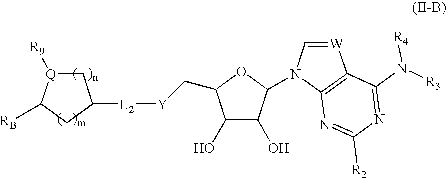

and pharmaceutically acceptable salts and complexes thereof, wherein
m is 0 or 1;
n is 1 or 2;
L₂ is a bond or is —CH₂—;
R₂ is H or halogen;
R₃ is H, C₁-C₆ alkyl, or —C₁-C₆-alkyl-aryl, wherein C₁-C₆ alkyl and aryl, at each occurrence, are optionally substituted;
P4 is H or C₁-C₆ alkyl, wherein C₁-C₆ alkyl is optionally substituted;
R₈ is H, —CO₂H, or CO₂CH₃;
R₉ is absent, H or C₁-C₆ alkyl, wherein C₁-C₆ alkyl is optionally substituted;
W is N or CH;
Y is S or O; and
Q is N, CH or O, provided that when Q is O, R₉ is absent.

In a preferred embodiment of the compounds according to paragraph [0084], R₃ and R₄ are both H.

In a preferred embodiment of the compounds according to paragraph [0084], R₃ is —C₁-C₆-alkyl-aryl and R₄ is H, wherein aryl is optionally substituted.

In a preferred embodiment of the compounds according to paragraph [0086], aryl is phenyl. Preferably, phenyl is unsubstituted or is substituted with phenyl.

In a preferred embodiment of the compounds according to paragraphs [0084]-[0087],

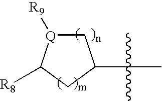

represents a pyrrolidinyl, piperidinyl, or azetidinyl ring, wherein said ring is unsubstituted or is substituted with COOH or COOCH₃.

In a preferred embodiment of the compounds according to paragraph [0048], the compounds are of formula II-C;

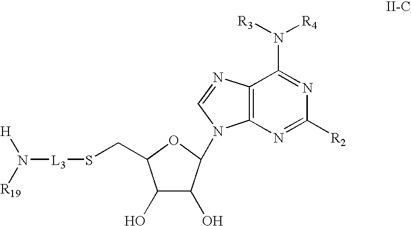

and pharmaceutically acceptable salts and complexes thereof, wherein
R₂ is H, or halogen;
R₃ and R₄ independently represent H, C₁-C₆ alkyl, C₂-C₆ alkenyl, C₂-C₆ alkynyl, cycloalkyl, aryl or (—C₁-C₆-alkyl)-aryl, wherein C₁-C₆ alkyl, C₂-C₆ alkenyl, C₂-C₆ alkynyl, cycloalkyl, aryl and (—C₁-C₆-alkyl)-aryl are each optionally substituted; or
   R₃ and R₄ taken together with the nitrogen to which they are attached form a C₅-C₉-heterocyclyl ring or a heteroaryl ring, wherein said ring is optionally substituted;
L₃ is a bond or is —(CR₁₇R₁₈)₁₋₆—;
   R₁₇ and R₁₈ at each occurrence are independently H or C₁-C₆-alkyl, wherein C₁-C₆-alkyl is optionally substituted;
R₁₉ is H, C₁-C₆-alkyl, heteroaryl, or H₂N—C(═NH)—CH₂—, wherein C₁-C₆-alkyl and heteroaryl are optionally substituted.

In a preferred embodiment of the compounds according to paragraph [0089], L₃ is —CHR₁₇CHR₁₈—, wherein R₁₇ and R₁₈ independently are H, or C₁-C₆ alkyl, and where C₁-C₆ alkyl at each occurrence is optionally substituted. Preferably, C₁-C₆ alkyl is unsubstituted or is substituted with NH₂.

In a preferred embodiment of the compounds according to paragraph [0089], L₃ is —CH₂CH₂CH₂—.

In a preferred embodiment of the compounds according to paragraphs [0089]-[0091], R₁₉ is H.

In a preferred embodiment of the compounds according to paragraphs [0089]-[0091], R₁₉ is C₁-C₆ alkyl, wherein C₁-C₆ alkyl is optionally substituted. Preferably, C₁-C₆ alkyl is unsubstituted or is substituted with NH₂.

In a preferred embodiment of the compounds according to paragraphs [0089]-[0091], R₁₉ is H₂N—C(═NH)—CH₂—.

In a preferred embodiment of the compounds according to paragraphs [0089]-[0091], R₁₉ is heteroaryl.

In a preferred embodiment of the compounds according to paragraph [0095], heteroaryl is pyrimidin-2(1H)-one. Preferably, pyrimidin-2(1H)-one is unsubstituted or is substituted with amine.

In a preferred embodiment, the compounds listed in Table 1 are excluded from the compounds of paragraphs [0047]-[0096]:

TABLE 1

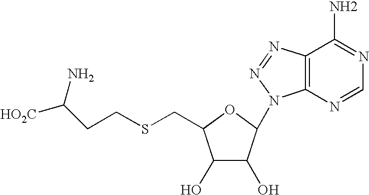

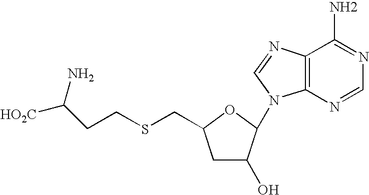

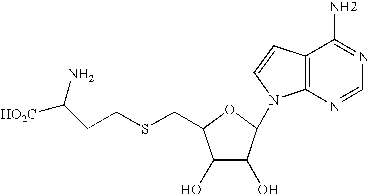

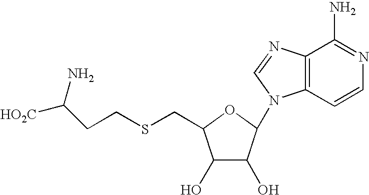

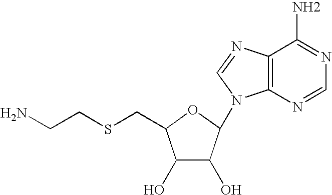

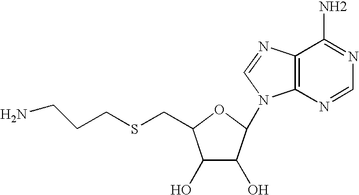

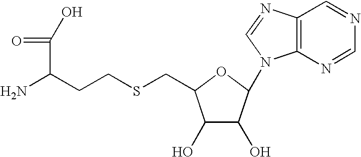

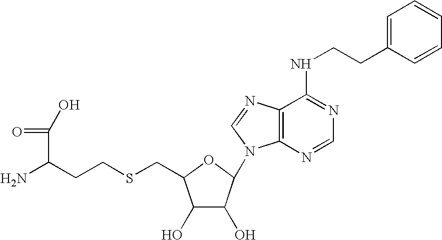

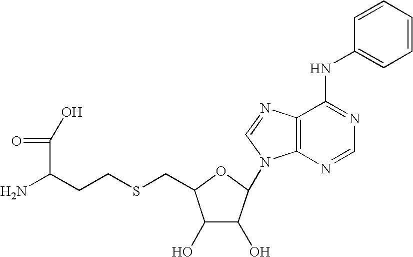

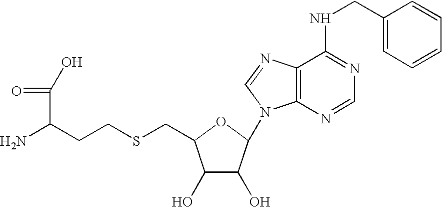

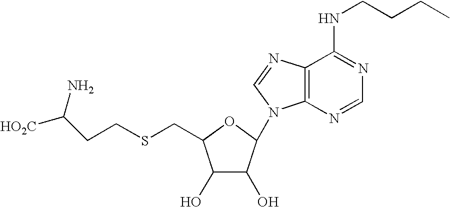

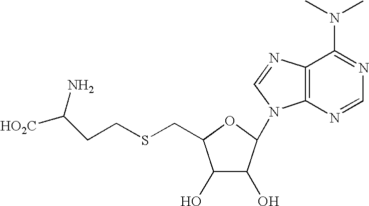

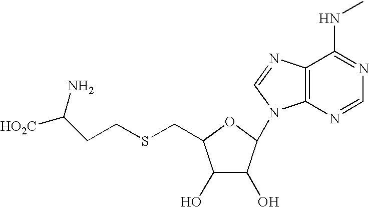

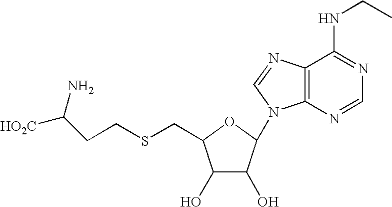

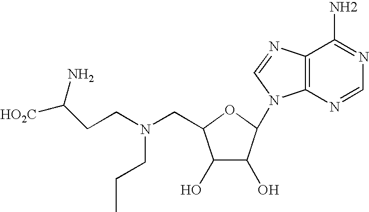

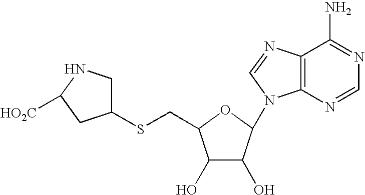

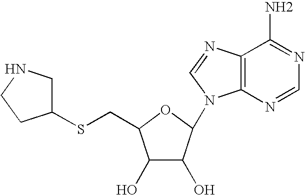

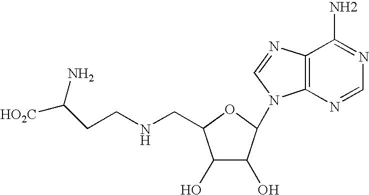

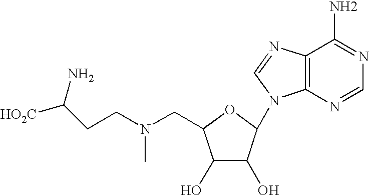

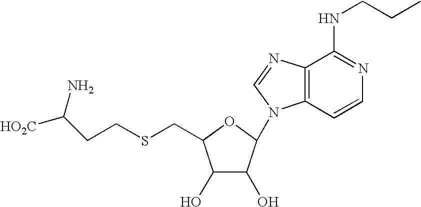

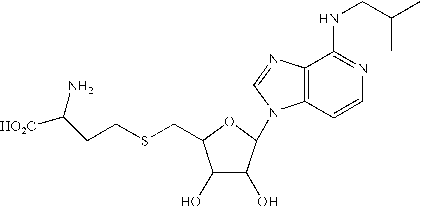

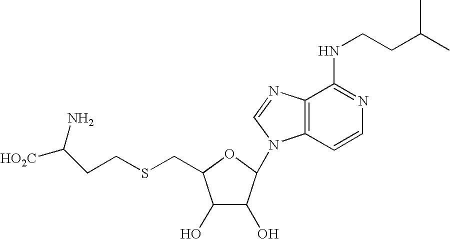

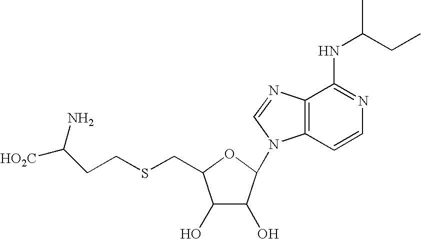

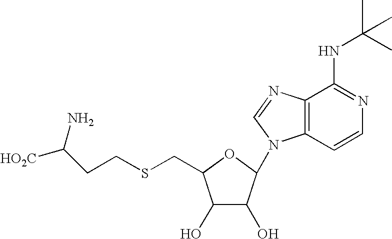

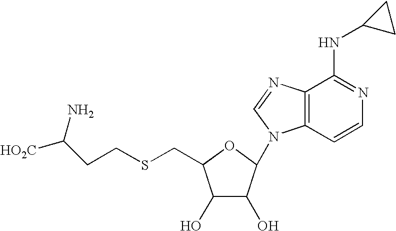

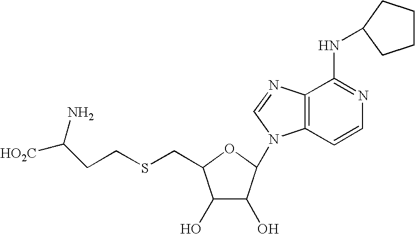

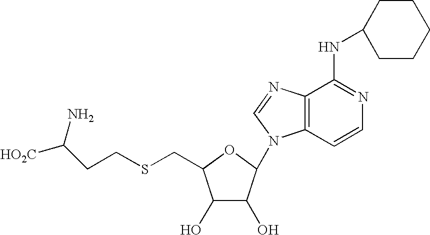

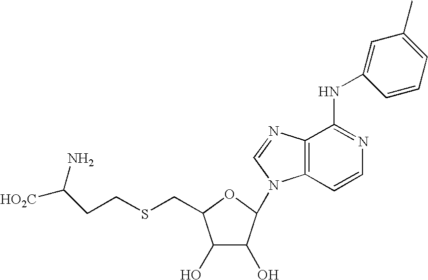

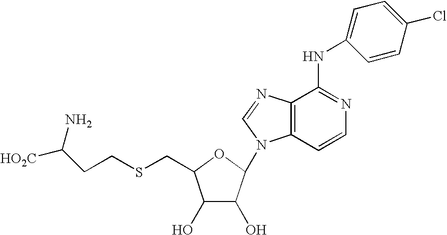

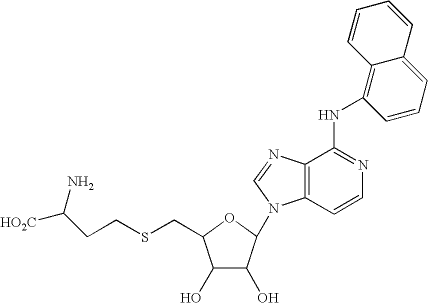

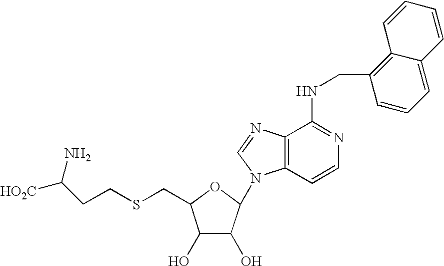

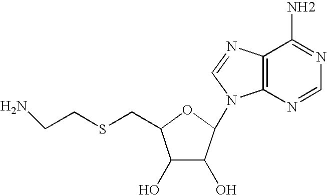

### Pharmaceutical Compositions

In a second aspect, the invention provides a composition comprising a compound according to any one of paragraphs [0047]-[0096] or as depicted in any of the examples and tables herein together with a pharmaceutically acceptable excipient, diluent, or carrier.

Compounds of the invention may be formulated by any method well known in the art.

As used herein, the term “pharmaceutically acceptable” means a non-toxic material that is compatible with a biological system in a cell, cell culture, or tissue sample and that does not interfere with the effectiveness of the biological activity of the active ingredient(s). Thus, compositions according to the invention may contain, in addition to the inhibitor, diluents, fillers, salts, buffers, stabilizers, solubilizers, and other materials well known in the art. The preparation of pharmaceutically acceptable formulations is described in, e.g., Remington's Pharmaceutical Sciences, 18th Edition, ed. A. Gennaro, Mack Publishing Co., Easton, Pa., 1990.

As used herein, the term pharmaceutically acceptable salts refers to salts that retain the desired biological activity of the above-identified compounds and exhibit minimal or no undesired toxicological effects. Examples of such salts include, but are not limited to acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, polyglutamic acid, naphthalenesulfonic acid, naphthalenedisulfonic acid, and polygalacturonic acid. The compounds can also be administered as pharmaceutically acceptable quaternary salts known by those skilled in the art, which specifically include the quaternary ammonium salt of the formula —NR+Z-, wherein R is hydrogen, alkyl, or benzyl, and Z is a counterion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate). As used herein, the term “salt” is also meant to encompass complexes, such as with an alkaline metal or an alkaline earth metal.

The active compound is included in the pharmaceutically acceptable carrier or diluent in an amount sufficient to deliver to an enzyme-inhibiting effective amount without causing serious toxic effects to the cell.

### Methods of Inhibiting DNMT1 and/or DNMT3b2 Enzymes

The third aspect of the invention provides a method of inhibiting DNMT1 and/or DNMT3b2 enzymes, the method comprising contacting the enzyme(s) with a compound according to any one of paragraphs [0047]-[0097], or as depicted in any of the tables herein, or with a composition according to paragraph [0098]-[0102]. Inhibition of DNMT1 and/or DNMT3b2 enzymes can be in a cell or a multicellular organism. If in a multicellular organism, the method according to this aspect of the invention comprises administering to the organism an effective DNMT1- and/or DNMT3b2-inhibiting amount of a compound according to any one of paragraphs [0047]-[0097] or as depicted in any of the tables herein, or a composition according to paragraph [0098]-[0102]. Preferably the organism is a mammal, more preferably a primate, most preferably a human.

Preferred compounds according to the invention include those described in the examples below. Compounds were named using Chemdraw Ultra version 6.0.2 or version 8.0.3, which are available through Cambridgesoft.com, 100 Cambridge Park Drive, Cambridge, Mass. 02140, Namepro version 5.09, which is available from ACD labs, 90 Adelaide Street West, Toronto, Ontario, M5H, 3V9, Canada, or were derived therefrom.

### Synthetic Schemes and Experimental Procedures

The compounds of the invention can be prepared according to the reaction schemes or the examples illustrated below utilizing methods known to one of ordinary skill in the art. These schemes serve to exemplify some procedures that can be used to make the compounds of the invention. One skilled in the art will recognize that other general synthetic procedures may be used. The compounds of the invention can be prepared from starting components that are commercially available. Any kind of substitutions can be made to the starting components to obtain the compounds of the invention according to procedures that are well known to those skilled in the art.

Compounds belonging to this class were prepared according to scheme 1a as detailed for example 1.

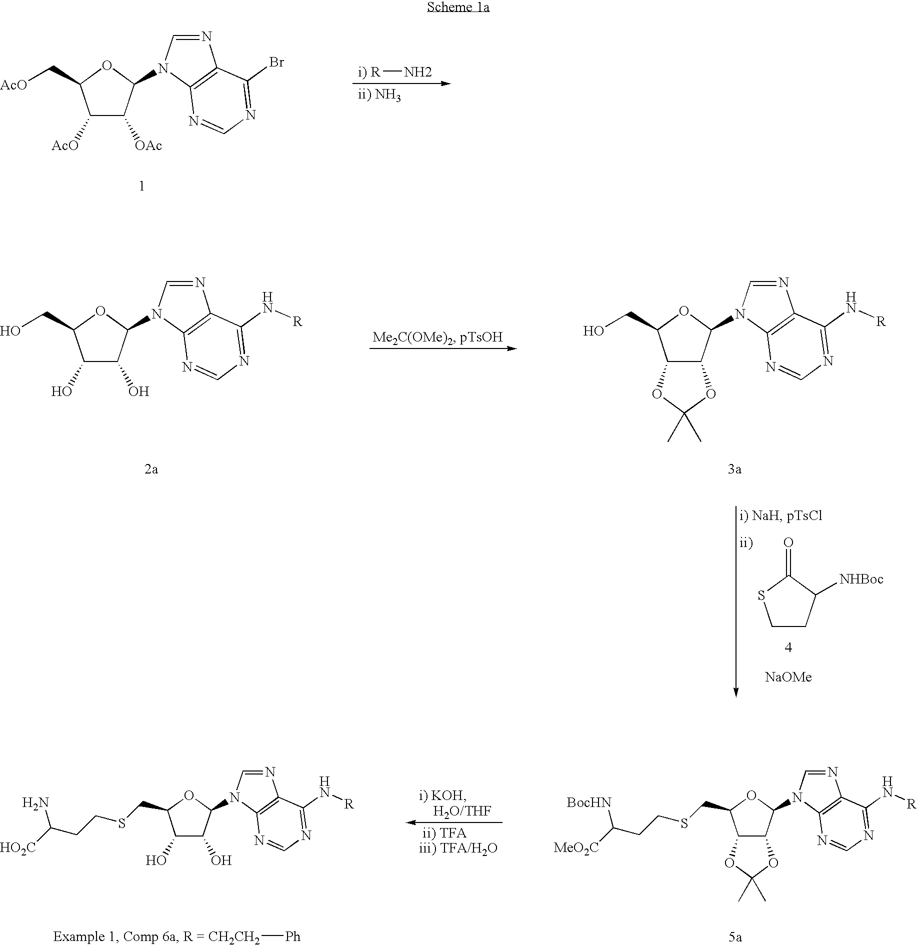

### EXAMPLE 1

### 2-AMINO-4-(((2S,3S,4R,5R)-3,4-DIHYDROXY-5-(6-(PHENETHYLAMINO)-9H-PURIN-9-YL)-TETRAHYDROFURAN-2-YL)METHYLTHIO)BUTANOIC ACID 6a

### Step1: (2R,3S,4R,5R)-2-(hydroxymethyl)-5-(6-(phenethylamino)-9H-purin-9-yl)-tetrahydrofuran-3,4-diol 2a

A solution of 2a (220 mg, 0.48 mmol, prepared according to the method of E. A. Veliz, et al., Tet. Lett. 2000, 41, 1695) and phenylethyl amine (182 μL, 175 mg, 1.45 mmol) in DME (5 mL) was stirred for 4 hours at room temperature. The reaction mixture was then diluted in EtOAc (15 mL) and washed with NaCl sat solution (15 mL), dried with Na₂SO₄, filtered and concentrated in vacuo. The crude product was stirred in saturated methanolic solution of ammonia at room temperature for overnight. The mixture was concentrated in vacuo and purified by flash chromatography using 5% MeOH in DCM as the eluent giving the title compound 2a in 94% yield as white solid (168 mg). MS: calc 371; found 372 (MH⁺).

### Step 2: ((3aR,4R,6R,6aR)-2,2-dimethyl-6-(6-(phenethylamino)-9H-purin-9-yl)-tetrahydrofuro[3,4-d][1,3]dioxol-4-yl)methanol 3a

A solution of (2R,3S,4R,5R)-2-(hydroxymethyl)-5-(6-(phenethylamino)-9H-purin-9-yl)-tetrahydrofuran-3,4-diol 2a (168 mg, 0.45 mmol), 2,2-dimethoxypropane (300 μL, 250 mg, 2.4 mmol) and pTsOH (100 mg, 0.52 mmol) in acetone (5 mL) was stirred at room temperature for 3 hrs. The reaction was quenched by the addition of Et₃N (200 μL) and concentrated in vacuo. The crude product was purified by flash chromatography using the gradient eluent from 70-100% EtOAc in hexanes giving the title compound 3a in 81% yield (149 mg) white solid MS: calo 411; found 412 (MH⁺).

### Step 3: methyl 2-(tert-butoxycarbonylamino)-4-(((3aS,4S,6R,6aR)-2,2-dimethyl-6-(6-(phenethylamino)-9H-purin-9-yl)-tetrahydrofuro[3,4-d][1,3]dioxol-4-yl)methylthio)butanoate 5a

NaH (30 mg 60% mineral oil suspension, 0.72 mmol) was added to a solution of ((3aR,4R,6R,6aR)-2,2-dimethyl-6-(6-(phenethylamino)-9H-purin-9-yl)-tetrahydrofuro[3,4-d][1,3]dioxol-4-yl)methanol 3a (149 mg, 0.36 mmol) in THF (4 mL) at 0° C. and stirred for 15 min. pTsCl (76 mg, 0.4 mmol) was then added and the reaction mixture was allowed to stir for 1 hour at 0°C. It was diluted in EtOAc (15 mL) and washed sequentially with water (10 mL) and NaCl sat solution (10 mL). The organic layer was dried with Na₂SO₄, filtered and concentrated in vacuo. The crude product was dissolved in dry MeOH and treated with a solution prepared by mixing tert-butyl 2-oxo-tetrahydrothiophen-3-ylcarbamate 4 (114 mg, 0.52 mmol) and NaOMe (1.05 mL 0.5M solution, 0.52 mmol) for 15 min at room temperature. The resultant reaction mixture was heated at reflux for 4 hours. It was allowed to cool to room temperature and concentrated in vacuo. The mixture was diluted in EtOAc (10 mL) and washed with water (5 mL) and sat'd NaCl solution (5 mL). The organic layer was dried with Na₂SO₄, filtered and concentrated in vacuo. The crude product was purified by flash chromatography using the gradient from 50-70% EtOAc in hexanes as the eluent giving the title compound 5a in 43% yield (97 mg) white solid, MS: calc 642; found 643 (MH⁺).

### Step 4: 2-amino-4-(((2S,3S,4R,5R)-3,4-dihydroxy-5-(6-(phenethylamino)-9H-purin-9-yl)-tetrahydrofuran-2-yl)methylthio)butanoic acid 6a

A solution of methyl 2-(tert-butoxycarbonylamino)-4-(((3aS,4S,6R,6aR)-2,2-dimethyl-6-(6-(phenethylamino)-9H-purin-9-yl)-tetrahydrofuro[3,4-d][1,3]dioxol-4-yl)methylthio)butanoate 5a (97 mg, 0.15 mmol) and KOH (40 mg, 0.71 mmol) in 1:1 mixture of THF and water (3 mL) was stirred at room temperature for 2 hours. The reaction mixture was then concentrated in vacuo and diluted in water (5 mL). It was washed with Et₂O (5 mL) and acidified to pH=2 by addition of citric acid. The aqueous phase was then extracted with EtOAc (3×5 mL) and the combined organic layer was dried with Na₂SO₄, filtered and concentrated in vacuo.

The crude product above was treated with 2:1 mixture of TFA/water (3 mL) containing trace amount of BHT. The reaction mixture was stirred at room temperature for 45 min and then it was treated with 5 drops of water. It was allowed to stir for another 1 hour before it was concentrated in vacuo. The title compound 6a was obtained in 40% yield (29 mg) after purification by flash chromatography using the gradient from 30% MeOH/DCM to 60:30:10 CHCl₃/MeOH/NH₄OH. ¹H NMR (DMSO-d₆)) δ8.26 (s, 1H), 8.16 (s, 1H), 7.79 (br.s, 1H), 7.0-7.2 (m, 5H), 5.80 (d, 1H, J=5.6 Hz), 4.64 (m, 1H), 4.08 (m, 1H), 3.94 (m, 1H), 3.61 (br.s, 2H), 2H assumed under H₂O at 3.33, 2.83 (t, 2H, J=7.5 Hz), 2.71 (m, 1H), 2.54 (m, 2H), 1.90 (m, 1H), 1.73 (m, 1H). MS: calc 488; found 489 (MH⁺).

Examples 2-9, compounds 5b-5i, Table 2, were prepared in a manner similar to example 1, scheme 1a, utilizing the appropriate amine.

Alternatively, compounds belonging to this class were prepared as illustrated in scheme 1b, example 10, compound 12a.

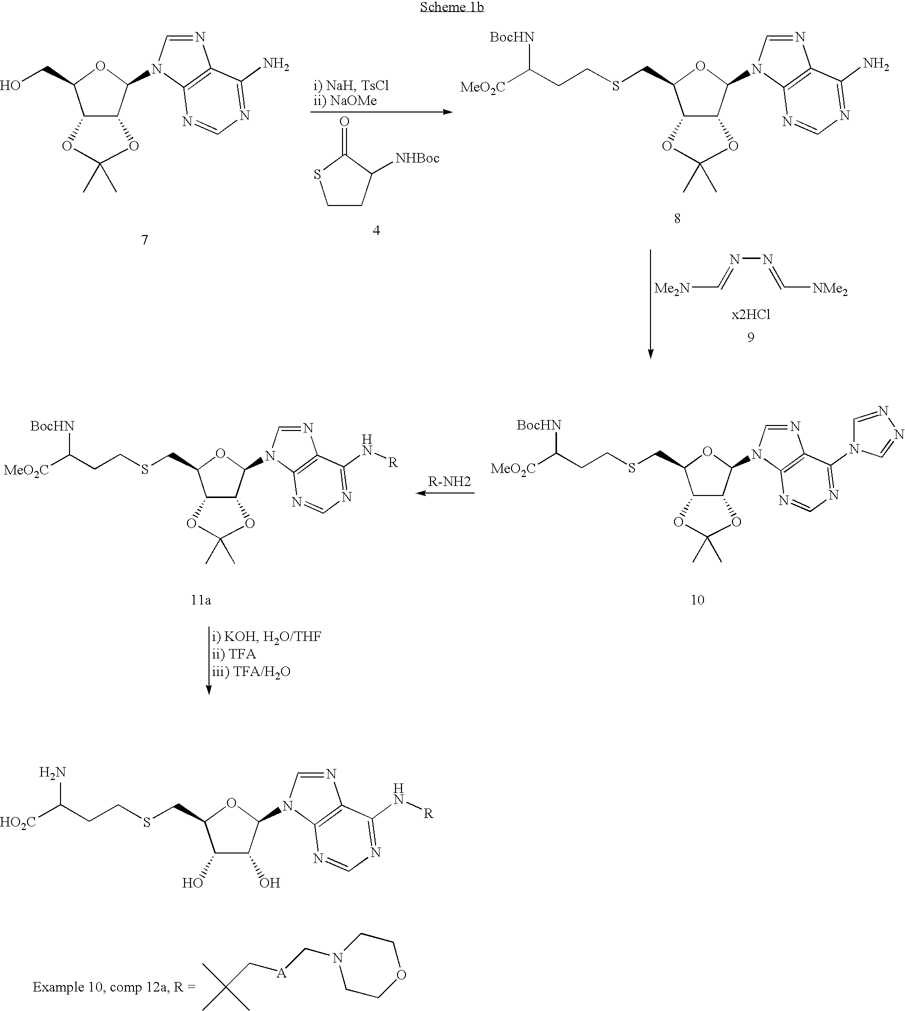

### EXAMPLE 10

### 2-amino-4-(((2S,3S,4R,5R)-3,4-dihydroxy-5-(6-(3-morpholinopropylamino)-9H-purin-9-yl)-tetrahydrofuran-2-yl)methylthio)butanoic acid 12a

### Step 1: methyl 4-(((3aS,4S,6R,6aR)-6-(6-amino-9H-purin-9-yl)-2,2-dimethyl-tetrahydrofuro[3,4-d][1,3]dioxol-4-yl)methylthio)-2-(tert-butoxycarbonylamino)butanoate 8

NaH (310 mg 60% mineral oil suspension, 7.74 mmol) was added to a solution of ((3aR,4R,6R,6aR)-6-(6-amino-9H-purin-9-yl)-2,2-dimethyl-tetrahydrofuro[3,4-d][1,3]dioxol-4-yl)methanol 7 (1.19 g, 3.87 mmol) in THF (4 mL) at 0° C. and stirred for 15 min. pTsCl (810 mg, 4.25 mmol) was then added and the reaction mixture was allowed to stir for 1 hour at 0° C. It was diluted in EtOAc (15 mL) and washed sequentially with water (10 mL) and NaCl sat solution (10 mL). The organic layer was dried with Na₂SO₄, filtered and concentrated in vacuo, dissolved in MeOH and added to a pre-formed solution of thiolactone 4 (1.26 g, 5.81 mmol, 1.5 equiv.) in dry MeOH (5 mL) was treated with 0.5 M solution of NaOMe (11.6 mL, 5.81 mmol, 1.5 equiv.) at room temperature and stirred for 15 min. The mixture was refluxed for 2 hours, and then it was cooled down and concentrated. The residue was diluted in EtOAc (20 mL), washed with water (20 mL) and sat'd NaCl solution (20 mL), dried with Na₂SO₄, filtered and concentrated in vacuo. The crude product was purified by flash chromatography using 5% MeOH in DCM as the eluent to afford the title compound 8 in 59% yield as white solid (1.27 g)

MS: calc 538; found 539 (MH⁺)

### Step 2: methyl 4-(((3aS,4S,6R,6aR)-6-(6-(4H-1,2,4-triazol-4-yl)-9H-purin-9-yl)-2,2-dimethyl-tetrahydrofuro[3,4-d][1,3]dioxol-4-yl)methylthio)-2-(tert-butoxycarbonylamino)butanoate 10

The dihydrochloride salt 9 (398 mg, 1.85, 5 equiv.) was added to a solution of 8 (134 mg, 0.37 mmol) in dry pyridine (4 mL) and the reaction mixture was refluxed for 16 h. It was then cooled to room temperature and concentrated in under reduced pressure. Crude material was purified by flash chromatography using EtOAc as the eluent to afford the title compound 10 in 55% yield (80 mg). MS: calc 590; found 591 (MH⁺)

### Step 3: methyl 2-(tert-butoxycarbonylamino)-4-(((3aS,4S,6R,6aR)-2,2-dimethyl-6-(6-(3-morpholinopropylamino)-9H-purin-9-yl)-tetrahydrofuro[3,4-d][1,3]dioxol-4-yl)methylthio)butanoate 11a

A solution of 10 (45 mg, 0.085 mmol, 1 equiv.) and 3-morpholinopropan-1-amine (37 μL, 36 mg, 0.25 mmol, 3 equiv.) in dry DMF (7 mL) was stirred at room temperature for 2 days. The reaction mixture was concentrated, diluted in EtOAc (20 mL) and washed with sat'd NaCl solution (10 mL). Organic phase was dried with Na₂SO₄, filtered and concentrated. The crude material was purified by flash chromatography using EtOAc as the eluent to give the title compound 11a in 30% yield (15 mg). MS: calc 665; found 666 (MH⁺)

### Step 4: 2-amino-4-(((2S,3S,4R,5R)-3,4-dihydroxy-5-(6-(3-morpholinopropylamino-9H-purin-9-yl)-tetrahydrofuran-2-yl)methylthio)butanoic acid 12a

The deprotection was carried out similar to example 1, scheme 1a, step 4 described earlier to give the title compound as white solid. ¹H NMR (D₂O) δ8.24 (s, 1H), 8.17 (s, 1H), 6.01 (d, 1H, J=5.3 Hz), 4.38 (m, 1H), 4.29 (m, 1H), 3.78 (m, 6H), 3.58 (m, 2H), 2.9-3.1 (m, 2H), 2.5-2.8 (m, 8H), 1.8-2.2 (m, 4H)

MS: calc 511; found 512 (MH⁺).

Examples 11-19, compounds 12b-12j, Table 2, were prepared similar to example 10, scheme 1b, by reacting compound 10 with the appropriate commercial amine.

TABLE 2

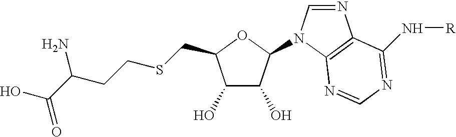

| Ex | Cpd | R | Name | Characterization | Scheme |
|---|---|---|---|---|---|
| 2 | 6b | 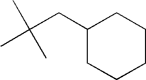 | 2-amino-4-(((2S,3S,4R,5R)-5-(6-(cyclo-hexylmethylami-no)-9H-purin-9-yl)-3,4-di-hydroxy-tetra-hydrofuran-2-yl)methyl-thio)butanoicacid | ¹H NMR(DMSO-d₆) δ8.30(s, 1H),8.16(s, 1H), 7.82(br.s, 1H), 5.84(d,1H, J=5.9Hz), 4.69(br.s, 1H), 4.13(d,1H, J=7.2Hz), 3.98(m, 1H), 2.87(m,1H), 2.75(m, 1H),2.59(m, 2H), 2.47(s,1H), 1.95(m, 1H), 1.79(m, 1H),0.9–1.7(m, 13H)MS: calc 480, found 481(MH⁺) | 1a |
| 3 | 6c | 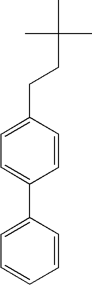 | 2-amino-4-(((2S,3S,4R,5R)-5-(6-(2-(bi-phenyl-4-yl)ethyl-amino)-9H-pur-in-9-yl)-3,4-di-hydroxy-tetrahydrofuran-2-yl)methyl-thio)butanoicacid | ¹H NMR(DMSO-d₆) δ8.38(s, 1H),8.27(s, 1H), 7.93(br.s, 1H), 7.63(d,2H, J=8.4Hz), 7.58(d, 2H, J=8.0Hz),7.44(t, 2H, J=7.4Hz), 7.35(m,3H),5.91(d, 1H, J=5.7Hz), 4.75(t, 1H,J=5.3Hz), 4.20(m, 1H), 4.05(m, 1H),2.9–3.0(m, 4H), 2.83(m, 2H), 2.64(t,2H, J=7.6Hz), 2.02(m, 1H), 1.84(m,1H). MS: calc 564; found 565(MH⁺) | 1a |
| 4 | 6d | 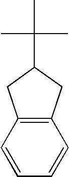 | 2-amino-4-(((2S,3S,4R,5R)-5-(6-(2,3-di-hydro-1H-in-den-2-ylamino)-9H-pur-rin-9-yl)-3,4-di-hydroxy-tetrahydrofuran-2-yl)methyl-thio)butanoicacid | ¹H NMR(DMSO-d₆) δ8.34(s, 1H),8.29(s, 1H), 8.01(br.s, 1H), 7.15(m,2H), 7.05(m, 2H), 5.87(d, 1H, J=5.6Hz),4.88(br.s, 1H), 4.08(m,1H), 3.95(m,1H), 2.8–3.0(m, 6H),2.73(m, 2H), 2.54(m, 2H), 1.90(m,1H), 1.71(m, 1H). MS: calc 500; found501(MH⁺) | 1a |
| 5 | 6e | 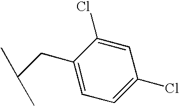 | 2-amino-4-(((2S,3S,4R,5R)-5-(6-(2,4-di-chlorobenzylamino)-9H-pur-in-9-yl)-3,4-di-hydroxy-tetra-hydrofuran-2-yl)methyl-thio)butanoicacid | ¹H NMR(DMSO-d₆) δ8.49(s, 1H),8.27(s, 1H), 7.65(m, 2H), 5.97(d, 1H,J=5.2Hz), 4.22(m, 1H), 4.09(m, 1H),3.39(m, 1H), 3.22(s, 1H), 2.96(m,1H),2.87(m, 1H), 2.68(m, 1H), 2.56(s,1H), 2.46(m, 2H), 2.04(m, 1H),1.9(m, 1H). MS: calc 542; found 543(MH⁺) | 1a |
| 6 | 6f | 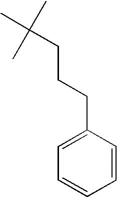 | 2-amino-4-(((2S,3S,4R,5R)-3,4-di-hydroxy-5-(6-(3-phenyl-propylamino)-9H-pur-in-9-yl)-tetra-hydrofuran-2-yl)methyl-thio)butanoicacid | ¹H NMR(DMSO-d₆) δ8.32(s, 1H),8.18(br.s, 1H), 7.89(br.s, 1H), 7.09–7.23(m,5H), 5.86(d, 1H, J=5.9Hz),4.70(m, 1H), 4.14(m, 1H), 4.05(m,1H),3.3–3.5(m, 3H), 2.90(m, 1H),2.77(m, 1H), 2.60(t, 4H, 7.6Hz), 1.98(m,1H), 1.86(m, 3H). MS: calc 502;found 503(MH⁺) | 1a |
| 7 | 6g | 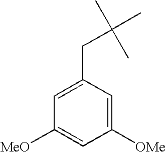 | 2-amino-4-(((2S,3S,4R,5R)-5-(6-(3,5-di-methoxybenzylamino)-9H-pur-in-9-yl)-3,4-di-hydroxy-tetra-hydrofuran-2-yl)methyl-thio)butanoicacid | ¹H NMR(DMSO-d₆) δ8.41(s, 1H),8.25(s, 1H), 6.48(s, 2H), 6.36(s, 1H),5.90(d, 1H, J=5.7Hz), 4.65(m, 1H),4.21(m, 1H), 4.03(m, 1H), 3.76(s,6H),3.01(m, 1H), 2.91(m, 1H), 2.6–2.7(, 2H),2.46(s, 2H), 2.04(m, 1H),1.95(m, 1H). MS: calc 534; found 535(MH⁺) | 1a |
| 8 | 6h | 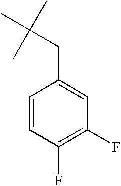 | 2-amino-4-(((2S,3S,4R,5R)-5-(6-(3,4-di-fluorobenzylamino)-9H-pur-in-9-yl)-3,4-di-hydroxy-tetra-hydrofuran-2-yl)methyl-thio)butanoicacid | ¹H NMR(DMSO-d₆) δ8.38(s, 1H),8.19(s, 1H), 7.30(m, 2H), 7.14(m,1H), 5.88(d, 1H, J=5.9Hz), 4.71(m,1H), 4.64(br.s, 1H), 4.15(m, 1H), 4.01(m,1H),2.92(m, 1H), 2.78(m, 1H),2.61(m, 2H), 1.97(m, 1H), 1.82(m,1H). MS: calc 510; found 511(MH⁺) | 1a |
| 9 | 6i | 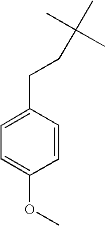 | 2-amino-4-(((2S,3S,4R,5R)-3,4-di-hydroxy-5-(6-(4-meth-oxyphenethylami-no)-9H-purin-9-yl)-tetra-hydrofuran-2-yl)methyl-thio)butanoicacid | ¹H NMR(D₂O) δ8.16(s, 1H), 8.05(s,1H), 7.04(d, 2H, J=8.4Hz), 6.69(d,2H, J=8.2Hz), 5.95(d, 1H, J=4.6Hz),4.34(m, 1H), 4.28(m, 1H),3.77(m,2H), 3.7(m, 1H), 3.68(s, 3H), 3.00(m,1H), 2.94(m, 1H), 2.82(m, 2H), 2.66(m,2H), 2.56(m, 1H), 2.0–2.2(m, 2H).MS: calc 518; found 519(MH⁺) | 1a |
| 11 | 12b | 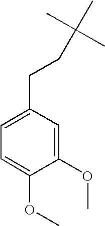 | 2-amino-4-(((2S,3S,4R,5R)-5-(6-(3,4-di-methoxyphenethyla-mino)-9H-purin-9-yl)-3,4-di-hydroxy-tetra-hydrofuran-2-yl)methyl-thio)butanoicacid | ¹H NMR(D₂O) δ8.18(s, 1H), 8.00(s,1H), 6.74(m, 1H), 6.71(m, 2H), 5.95(d,1H, J=5.6Hz), 4.34(m, 1H), 4.28(m,1H), 3.77(m, 2H), 3.7(m,1H),3.68(s, 3H), 3.59(s, 3H), 3.00(m,1H), 2.94(m, 1H), 2.82(m, 2H), 2.66(m,2H), 2.56(m, 1H), 2.0–2.2(m, 2H)MS: calc 548; found 549(MH⁺) | 1b |
| 12 | 12c | 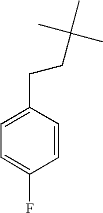 | 2-amino-4-(((2S,3S,4R,5R)-5-(6-(4-fluoro-phenethylamino)-9H-pur-in-9-yl)-3,4-di-hydroxy-tetrahydrofuran-2-yl)methyl-thio)butanoicacid | ¹H NMR(D₂O) δ8.40(s, 1H), 8.21(br.s,1H), 7.72(br.s, 2H), 7.46(d, 2H,J=8.6Hz), 6.07(d, 1H, J=4.9Hz), 4.41(m,1H), 4.32(m, 1H), 3.90(m,2H),3.16(m, 2H), 3.01(m, 3H), 2.84(d,1H, 7.0Hz), 2.70(m, 2H), 2.0–2.3(m,2H)MS: calc 506; found)507(MH⁺) | 1b |
| 13 | 12d | 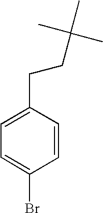 | 2-amino-4-(((2S,3S,4R,5R)-5-(6-(4-bromo-phenethylamino)-9H-pur-in-9-yl)-3,4-di-hydroxy-tetrahydrofuran-2-yl)methyl-thio)butanoicacid | ¹H NMR(D₂O) δ8.38(s, 1H), 8.21(br.s,1H), 7.34(br.s, 2H), 7.14(br.s,2H), 6.06(d, 1H, J=4.9Hz), 4.41(m,1H), 4.31(m, 1H), 3.89(m, 2H),2.9–3.1(m,5H), 2.81(d, 1H, J=15.6Hz),2.68(m, 2H), 2.0–2.2(m, 2H)MS: calc 566; found 567(MH⁺) | 1b |
| 14 | 12e | 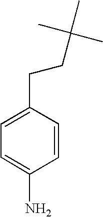 | 2-amino-4-(((2S,3S,4R,5R)-5-(6-(4-amino-phenethylamino)-9H-pur-in-9-yl)-3,4-di-hydroxy-tetrahydrofuran-2-yl)methyl-thio)butanoicacid | ¹H NMR(DMSO-d₆) 8.33(s, 1H), 8.23(s,1H), 7.78(br.s, 1H), 6.87(d, 2H,J=7.6Hz), 6.47(d, 2H, J=7.7Hz), 5.88(d,1H, J=5.9Hz), 4.73(m,1H), 4.15(m,1H), 4.01(m, 1H), 3.58(m, 2H),2.94(m, 1H), 2.81(m, 2H), 2.71(m,2H), 2.62(m, 2H), 1.98(m, 1H), 1.79(m,1H)MS: calc 503 found 504(MH⁺) | 1b |
| 15 | 12f | 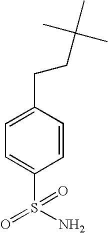 | 2-amino-4-(((2S,3S,4R,5R)-3,4-di-hydroxy-5-(6-(4-sulfa-moylphenethyla-mino)-9H-purin-9-yl)-teshe trahydrofuran-2-yl)meth-ylthio)butanoicacid | ¹H NMR(D₂O) δ8.19(s, 1H), 8.03(s,1H), 7.61(d, 2H, J=8.2Hz), 7.34(d,2H, J=8.2Hz), 5.96(d, 1H, J=5.1Hz),4.36(m, 1H), 4.28(m, 1H),3.7–3.9(m,3H), 3.0–3.1(m, 4H), 2.94(m, 1H),2.69(m, 2H), 2.0–2.2(m, 2H)MS: calc 567; found 568(MH⁺) | 1b |
| 16 | 12g | 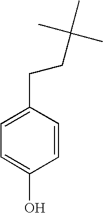 | 2-amino-4-(((2S,3S,4R,5R)-3,4-di-hydroxy-5-(6-(4-hydroxy-phenethylami-no)-9H-purin-9-yl)-tetra-hydrofuran-2-yl)meth-ylthio)butanoicacid | ¹H NMR(D₂O) δ8.15(s, 1H), 8.05(s,1H), 7.01(d, 2H, J=8.0Hz), 6.65(d,2H, J=7.9Hz), 5.96(d, 1H, J=4.9Hz),4.34(m, 1H), 4.28(m, 1H),3.69(m,3H), 3.33(s, 1H), 3.00(m, 1H), 2.92(m,1H), 2.80(m, 2H), 2.63(m, 2H),1.9–2.2(m, 2H)MS: calc 504; found 505(MH⁺) | 1b |
| 17 | 12h | 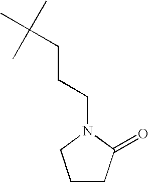 | 2-amino-4-(((2S,3S,4R,5R)-3,4-di-hydroxy-5-(6-(3-(2-oxo-pyrrolidin-1-yl)propyl-amino)-9H-pur-in-9-yl)-tetra-hydrofuran-2-yl)meth-ylthio)butanoicacid | ¹H NMR(DMSO-d₆) 8.34(s, 1H), 8.21(s,1H), 5.88(d, 1H, J=5.7Hz), 4.73(m,1H), 4.16(m, 1H), 4.02(m, 1H),3.2–3.5(m,7H), 2.94(m, 1H), 2.78(m,1H), 2.62(m, 2H), 2.23(m, 2H), 1.7–2.1(m,6H)MS: calc 509; found 510(MH⁺) | 1b |
| 18 | 12i | 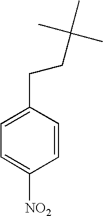 | 2-amino-4-(((2S,3S,4R,5R)-3,4-di-hydroxy-5-(6-(4-nitro-phenethylamino)-9H-pur-in-9-yl)-tetra-hydrofuran-2-yl)methyl-thio)butanoicacid | ¹H NMR(DMSO-d₆) 8.27(s, 1H), 8.16(s,1H), 8.05(d, 2H, J=8.6Hz), 7.87(br.s,1H), 7.44(d, 2H, J=8.4Hz), 5.81(d,1H, J=4.8Hz), 4.65(m, 1H),4.07(m,1H), 3.96(m, 1H), 3.69(m, 2H),1H assumed under H₂O at 3.33, 3.00(m,2H), 2.86(m, 1H), 2.73(m, 1H),2.55(m, 2H), 1.93(m, 1H), 1.74(m,1H)MS: calc 533; found 534(MH⁺) | 1b |
| 19 | 12j | 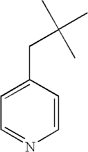 | 2-amino-4-(((2S,3S,4R,5R)-3,4-di-hydroxy-5-(6-(py-ridin-4-yl-methylamino)-9H-pur-in-9-yl)-tetra-hydrofuran-2-yl)methyl-thio)butanoicacid | ¹H NMR(DMSO-d₆) 8.1–8.5(m, 3H),8.17(s, 1H), 7.27(m, 2H), 5.88(d, 1H,J=5.5Hz), 4.71(m, 2H), 4.12(m, 1H),4.0(m, 1H), 2Hassumed under H₂O at3.33, 2.93(m, 1H), 2.79(m, 1H), 2.61(m,2H), 1.98(m, 1H), 1.84(m, 1H)MS: (calc) 475; (found) 476(M + H¹) | 1b |

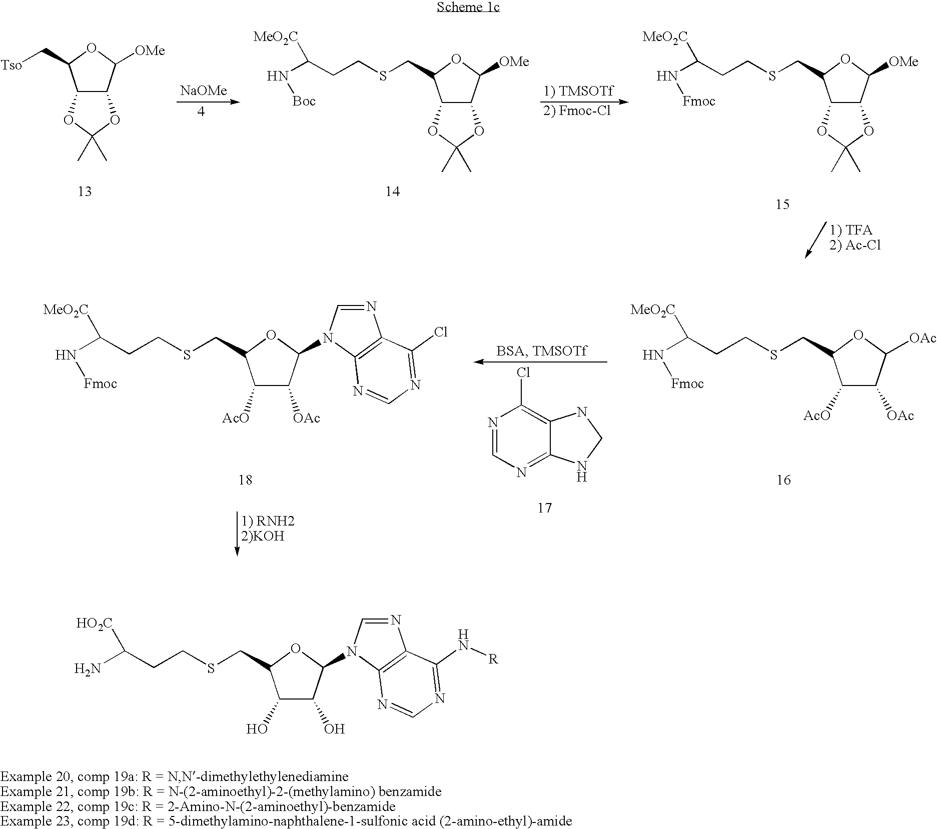

### EXAMPLE 20

### 2-amino-4-(((2S,3S,4R,5R)-5-(6-(2-(dimethylamino)ethylamino)-9H-purin-9-yl)-3,4-dihydroxy-tetrahydrofuran-2-yl)methylthio)butanoic acid 19a

### Step 1: methyl 2-(tert-butoxycarbonylamino)-4-(((3aS,4S,6R,6aR)-6-methoxy-2,2-dimethyl-tetrahydrofuro[3,4-d][1,3]dioxol-4-yl)methylthio)butanoate 14

A solution of N-tert-butoxycarbonyl-DL-homocysteine thiolactone 4 (9.1 g, 41.9 mmol) in NaOMe (0.5 M in methanol, 84 ml, 42 mmol) was stirred under a nitrogen atmosphere for 10 minutes. Methyl 2,3-O-isopropylidene-5-O-p-tolylsulfonyl-β-D-ribofuranoside 13 (10.0 g, 27.9 mmol) was then added and the mixture was reflux for 3 hours. After the reaction had cooled to room temperature, the solvent was evaporated and the crude material was added to ethyl acetate (200 ml). The ethyl acetate solution was washed with saturated NaHCO₃ (2×100 ml), 5% HCl (2×100 ml), and brine (100 ml). The ethyl acetate solution was then dried with MgSO₄, filtered and evaporated to give the crude product. The title compound 14 was obtained in 83% yield (10.1 g) after purification by flash chromatography using 25% ethyl acetate and 75% hexanes. MS:calc 435.53; found 458 (M+Na⁺)

### Step 2: methyl 2-(((9H-fluoren-9-yl)methoxy)carbonylamino)-4-(((3aS,4S,6R,6aR)-6-methoxy-2,2-dimethyl-tetrahydrofuro[3,4-d]1,3]dioxol-4-yl)methylthio)butanoate 15

A solution of 14 (11.0 g, 25.3 mmol) in dry 1,2-dichloroethane (50 ml) was placed under a nitrogen atmosphere and cooled to 0° C. TMSOTf (4.6 ml, 25.4 mmol) was then added and the reaction was stirred for 1.5 hours at 0° C. The reaction was then quenched with saturated NaHCO₃ (100 ml) and 9-fluorenylmethyl chloroformate (6.5 g, 25.2 mmol) was added slowly as a gas evolved. The bi-phasic mixture was stirred vigoursouly for 5 hours and then diluted with CH₂Cl₂ (50 ml). The organic layer was separated, washed with saturated NaHCO₃ (2×50 ml), 10% HCl (2×50 ml), and brine (50 ml). The organic layer was then dried with MgSO₄, filtered and evaporated and crude material was purified by flash chromatography using 30% ethyl acetate and 70% hexanes to give 15 in 85.8% yield (12.1 g) as a clear oil. MS: calc 557.66; found 580 (M+Na⁺)

### Step 3: (3R,4S,5S)-5-((3-(((9H-fluoren-9-yl)methoxy)carbonylamino-4-methoxy-4-oxobutylthio)methyl)-tetrahydrofuran-2,3,4-triyl triacetate 16

A solution of 15 (10 g, 17.9 mmol) in trifluoroacetic acid (20 ml) and H₂O (20 ml) was stirred at room temperature for 5 hours. H₂O (100 ml) was then added slowly and a white solid precipitated from the solution. After this mixture had stirred for 0.5 hours, the precipitate was filtered and dried under high vacuum. The precipitate was added to a solution of dry CH₂Cl₂ (100 ml) and dry pyridine (20 ml) under a nitrogen atmosphere and cooled to 0° C. Acetyl chloride (8.9 ml, 0.125 mol) was added slowly to this solution via syringe over a period of one hour and the reaction mixture was stirred overnight as it warmed to room temperature. The reaction was then quenched slowly with saturated NaHCO₃. The quenched reaction was stirred for an additional 0.5 hours and then the organic phase was separated, washed with saturated NaHCO₃ (2×100 ml) and brine (100 ml), dried with MgSO₄, filtered and evaporated and the crude material was purified by flash chromatography using 30% ethyl acetate and 70% hexanes to give the title compound 16 in 77.2% yield (8.7 g) as a mixture of α and β isomers (1:1.7 molar ratio respectively by ¹H NMR). MS: calc 629.67; found 652 (M+Na⁺)

### Step 4: (2S,3S,4R,5R)-2-((3-(((9H-fluoren-9-yl)methoxy)carbonylamino)-4-methoxy-4-oxobutylthio)methyl)-5-(6-chloro-9H-purin-9-yl)-tetrahydrofuran-3,4-diyl diacetate 18

A solution of 6-chloropurine 17 (368 mg, 2.38 mmol) and N,O-bistrimethylsilylacetomide (588 μl, 2.38 mmol) in dry acetonitrile (10 ml) was stirred for ½ hour at room temperature under an N₂ atmosphere. 16 (1.0 g, 1.59 mmol), in dry acetonitrile (10 ml), was then added followed by TMSOTf (287 μl, 1.59 mmol) and the mixture was refluxed for ½ hour. After the reaction had cooled to room temperature, it was poured into saturated NaHCO₃ (100 ml) and extracted into ethyl acetate. The organic phase was then washed With brine (50 ml), dried with Na₂SO₄, filtered and evaporated and the crude material was purified by flash chromatography using 95% CH₂CO₂ and 5% acetone to give the title compound 18 in 78.2% yield (900 mg).

### Step 5: 2-amino-4-(((2S,3S,4R,5R)-5-(6-(2-(dimethylamino)ethylamino)-9H-purin-9-yl)-3,4-dihydroxy-tetrahydrofuran-2-yl)methylthio)butanoic acid 19a

A solution of 18 (75 mg, 0.104 mmol) and N,N′-dimethylethylenediamine (36.5 mg, 0.414 mmol) in 3 ml of ethanol was refluxed for 2 days. The solvent was then evaporated and THF (0.5 ml) was added followed by 1 N KOH 1(0.5 ml). After stirring for 3 hours, the solvent was evaporated and the product was purified by flash chromatography using 60% CHCl₃, 30% methanol and 10% NH₄OH. The title compound 19a was obtained in 31.7% yield after trituration with CH₃CN (15 mg). ¹H NMR (DMSO-d₆/D₂O) δ ppm: 8.32 (d, 1H, J=2.4 Hz), 8.20 (s, 1H), 5.86 (d, 1H, J=5.2 Hz), 4.72 (m, 1H), 4.12-4.14 (m, 1H), 4.00 (m, 1H), 3.59 (bs, 2H), 3.16-3.23 (m, 1H), 2.75-2.92 (m, 2H), 2.54-2.61 (m, 4H), 2.28 (s, 6H), 1.91 (m, 1H), 1.76 (m, 1H). MS calc 455, found 456 (MH⁺)

### EXAMPLE 21

### 2-amino-4-(((2S,3S,4R,5R)-3,4-dihydroxy-5-(6-(2-(2-(methylamino)benzamido)ethylamino)-9H-purin-9-yl)-tetrahydrofuran-2-yl)methylthio)butanoic acid 19b

The title compound was prepared similar to example 20, in step 5 replacing N,N′-dimethylethylenediamine with N-(2-aminoethyl)-2-(methylamino)benzamide (prepared according to the method of Fassa, A. A.; Refat, H. M.; Zaki, M. E.;A.; Monir, E.; Synth. Commun. 2001, 31, 3537-3545). Compound 19b was obtained after flash chromatography using 60% CHCl₃, 30% methanol and 10% NH₄PH in 56.6% yield (33 mg). ¹H NMR (DMSO-d₆/D₂O) δ ppm:1.80 (m, 1H), 1.97 (m, 1H), 2.59 (t, 2H, J=7.6 Hz), 2.72-2.93 (m, 2H), 2.72 (s, 3H), 3.28 (m, 1H), 2H assumed under D₂O, 3.62 (m, 2H), 3.99 (m, 1H), 4.10-4.16 (m, 1H), 4.71 (dd, 1H, J=5.2, 5.2 Hz), 5.86 (d, 1H, J=5.6 Hz), 6.50 (m, 1H), 6.57 (d, 1H, J=7.6 Hz), 7.23 (m, 1H), 7.46 (d, 1H, J=7.6 Hz), 8.21 (s, 1H), 8.33 (s, 1H), 8.38 (bs, 1H). MS calc 560, found 561 (MH⁺).

### EXAMPLE 22

### 2-amino-4-(((2S,3S,4R,5R)-5-(6-(2-(2-aminobenzamido)ethylamino)-9H-purin-9-yl)-3,4-dihydroxy-tetrahydrofuran-2-yl)methylthio)butanoic acid 19c

The title compound was prepared similar to example 20, in step 5 replacing N,N′-dimethylethylenediamine with 2-amino-N-(2-aminoethyl)-benzamide. Compound 19c was obtained in 67.6% yield (37 mg) after flash chromatography using 60% CHCl₃, 30% methanol and 10% NH₄OH followed by recrystallisation from water and acetonitrile. ¹H NMR (DMSO-d₆/D₂O) δ ppm: 8.32 (s, 1H), 8.30 (bs, 1H), 8.21 (s, 1H), 7.41 (d, 1H, J=7.2 Hz), 7.09 (m, 1H), 6.64 (dd, 1H, J=1.2, 8.4 Hz), 6.46 (m, 1H), 5.86 (d, 1H, J=6.0 Hz), 4.71 (dd, 1H, J=5.2, 5.6 Hz), 4.10-4.17 (m, 1H), 4.00 (m, 1H), 3.62 (m, 2H), 3.45 (m, 2H), 3.27 (m, 1H), 2.75-2.95 (m, 2H), 2.59 (t, 2H, J=7.6 Hz), 1.97 (m, 1H), 1.81 (m, 1H). MS 546, found 547 (MH⁺).

### EXAMPLE 23

### 2-amino-4-(((2S,3S,4R,5R)-5-(6-(2-(5-(dimethylamino)naphthalene-1-sulfonamido)ethylamino)-9H-purin-9-yl)-3,4-dihydroxy-tetrahydrofuran-2-yl)methylthio)butanoic acid 19d

### Step 1: Tert-butyl 2-(1-(dimethylamino)naphthalene-5-sulfonamido)ethylcarbamate 20

A solution of tert-butyl-N(2-aminoethyl)carbamate (1.0 g, 6.24 mmol) and dansyl chloride (1.6 g, 5.93 mmol) was added to a mixture of acetonitrile (20 ml) and saturated NaHCO₃ (10 ml) according to the method of Corrie, John E. T. (J. Chem. Soc. Perkin. Trans. I, 1994, 20, 2975-2982). After the reaction was stirred for 30 minutes, ethyl acetate (100 ml) was then added and the organic phase was washed with saturated NaHCO₃ (2×50 ml) and brine (50 ml). The organic phase was then dried with MgSO₄, filtered and evaporated to give a crude oil which was purified by flash chromatography using 30% ethyl acetate and 70% hexanes to give the title compound 20 in 84.9% yield (1.98 g).

### Step 2: 5-dimethylamino-naphthalene-1-sulfonic acid (2-amino-ethyl)-amide 21

The protected amine 20 (1.0 g, 2.54 mmol) was added to a solution of CH₂Cl₂ (5 ml) and TFA (3 ml). The solution was stirred for 1 hour and then the solvent was evaporated. The crude material was then dissolved in water (30 ml) and neutralized with NaHCO₃, The aqueous phase was then extracted with ethyl acetate (4×50 ml) and the organic phases where combined, washed with brine (50 ml), dried with Na₂SO₄, filtered and evaporated. Precipitation from methylene chloride using hexanes gave title compound 21 in 47% yield (350 mg) as white solid.

### Step 3: 2-amino-4-(((2S,3S,4R,5R)-5-(6-(2-(5-(dimethylamino)naphthalene-1-sulfonamido)ethylamino)-9H-purin-9-yl)-3,4-dihydroxy-tetrahydrofuran-2-yl)methylthio)butanoic acid 19d

Amine 21 (91.5 mg, 0.312 mmol) was used in place of N,N′-dimethylethylenediamine and reacted with compound 18 (75 mg, 0.104 mmol) as described in example 20, step 5. After the reaction was refluxed overnight, 1.0 ml of ethanol was added before the reaction was refluxed for an additional 24 hours. The title compound 19d was obtained in 46.5% yield (32 mg) after flash chromatography using 60% CHCl₃, 30% methanol and 10% NH₄OH followed by recrystallization from CH₃CN. ¹H NMR (DMSO-d₆/D₂O) δ ppm: 8.37 (d, 1H, J=8.4 Hz), 8.30 (d, 1H, J=1.6 Hz), 8.18 (d, 1H, J=8.8 Hz), 8.11 (s, 1H), 8.05 (m, 1H), 7.53 (m, 1H), 7.46 (m, 1H), 7.15 (d, 1H, J=7.2 Hz), 5.85 (d, 1H, J=6.0 Hz), 4.70 (dd, 1H, J=5.2, 5.6 Hz), 4.10-4.16 (m, 1H), 4.00 (m, 1H), 2H assumed under D₂O, 3.26 (m, 1H), 2.87-2.98 (m, 4H), 2.78 (s, 6H), 2.59 (t, 2H, J=7.6 Hz), 1.98 (m, 1H), 1.81 (m, 1H). MS calc 660, found 661 (MH⁺).

Compounds belonging to this class, were prepared as illustrated in scheme 2, and detailed for example 24 (method A) and example 25 (method B) starting from the common intermediate 23

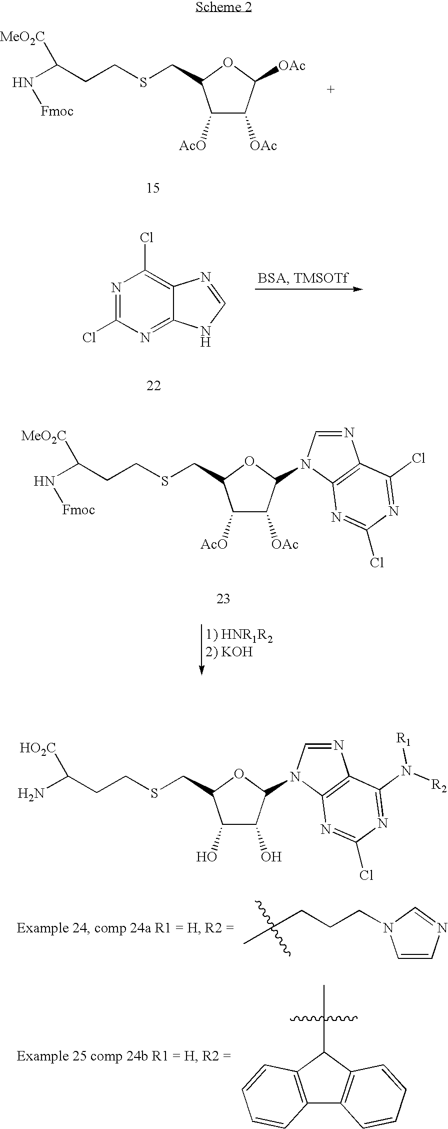

### Synthesis of the Common Intermediate (2S,3S,4R,5R)-2-((3-(((9H-fluoren-9-yl)methoxy)carbonylamino)-4-methoxy-4-oxobutylthio)methyl)-5-(2,6-dichloro-9H-purin-9-yl)-tetrahydrofuran-3,4-diyl diacetate 23

A solution of 2,6-dichloropurine 22 (164 mg, 0.868 mmol) and N,O-bistrimethylsilylacetomide (392 μl, 1.59 mmol) in dry CH₃CN (10 ml) was stirred under a nitrogen atmosphere for 0.5 hours. 15 (500 mg, 0.79 mmol), in dry CH₃CN (3 ml), and TMSOTf (144 μl, 0.794 mmol) were then added and the reaction was refluxed for 0.5 hours. After the reaction had cooled to room temperature, it was poured into 80 ml of saturated NaHCO₃ and extraced with ethyl acetate (2×80 ml). The organic layers were combined, washed with saturated NaHCO₃ (80 ml) and brine (80 mL), dried with Na₂SO₄, filtrated and evaporated. Purification of the crude product by flash chromatography using 95% CH₂Cl₂ and 5% acetone gave the title compound 23 in 81.8% yield (490 mg). ¹H NMR (DMSO-d₆) δ ppm: 8.93 (d, 1H, J=6.0 Hz), 7.86 (d, 2H, J=7.6), 7.76 (d, 1H, J=8.0 Hz), 7.67 (d, 2H, J=7.2 Hz), 7.39 (dd, 2H, J=7.2, 7.6 Hz), 7.30 (dd, 2H, J=7.2, 7.6 Hz), 6.27 (m, 1H), 5.94 (m, 1H), 5.55 (m, 1H), 4.35 (m, 1H), 4.29 (m, 2H), 4.21 (m, 1H), 4.10 (m, 1H), 3,59 (s, 3H), 3.03 (m, 2H), 2.56 (m, 2H), 2.12 (s, 3H), 2.02 (s, 3H), 1.89 (m, 2H). MS calc 757.14 (100%), found 780 (M+Na^{+).}

### EXAMPLE 24

### 4-(((2S,3S,4R,5R)-5-(6-(3-(1H-imidazol-1-yl)propylamino)-2-chloro-9H-purin-9-yl)-3,4-dihydroxy-tetrahydrofuran-2-yl)methylthio)-2-aminobutanoic acid 24a

### Method A

A solution of 23 (100 mg, 0.132 mmol) and 3-(1H-imidazol-1-yl)propan-1-amine (50 mg, 0.4 mmol) in dry 1,2-dichloroethane (3 ml) was stirred overnight at room temperature. The solvent was evaporated and THF (0.5 ml) was added followed by 1.0 M KOH (1.3 ml, 1.3 mmol). The solution was stirred for 3 to 5 hours and the solvent was removed to give the crude product which was purified by flash chromatograp using 70% CHCl₃, 25% methanol and 5% NH₄OH followed by recrystallized from a mixture of water and acetonitrile giving the title compound 24a in 75% yield as white solid (39 mg). ¹H NMR (DMSO-d₆/D₂O) δ ppm: 8.40 (s, 1H), 7.69 (s, 1H), 7.23 (s, 1H), 6.88 (s, 1H), 5.82 (d, 1H, J=5.6 Hz), 4.64 (m, 1H), 4.12 (m, 1H), 4.03 (m, 3H), 3.38 (m, 2H) and 3.80, 3.27 (m, 1H), 2.87 (m, 1H), 2.79 (m, 1H), 2.58 (m, 2H), 2.00 (m, 3H), 1.81 (m, 1H). MS calc 526, found 527 (MH⁺).

### EXAMPLE 25

### 4-(((2S,3S,4R,5R)-5-(6-(9H-fluoren-9-ylamino)-2-chloro-9H-purin-9-yl)-3,4-dihydroxy-tetrahydrofuran-2-yl)methylthio)-2-aminobutanoic acid 24b

### Method B

A solution of 23 (75 mg, 0.0989 mmol), triethylamine (83 μL, 6 eq) and 9-aminofluorene hydrochloride (64.6 mg, 0.3 mmol) in a mixture of 1,2-dichloroethane (0.75 ml) and ethanol (0.75 ml) was heated to 50° C.-60° C. overnight. The reaction was then cooled to room temperature and the solvent was evaporated. THF (0.5 ml) was added followed by 1.0 M KOH (1.5 ml). The solution was stirred for 3 hours and the solvent was removed to give the crude product which was purified by flash chromatography using 60% CHCl₃, 30% methanol and 10% NH₄OH followed by trituration with CH₃CN to give the title compound 24b in 57.7% yield(25 mg). ¹H NMR (DMSO-d₆/D₂O, 400 MHz): δ ppm: 8.38 (s, 1H), 7.84 (d, 2H, J=7.2 Hz), 7.25-7.48 (m, 4H), 7.25-7.31 (m, 2H), 6.50 (m, 1H), 5.84 (d, 1H, J=6.0 Hz), 4.65 (m, 1H), 4.10-4.16 (m, 1H), 4.05 (m, 1H), 3.22-3.37 (m, 1H), 2.77-2.96 (m, 2H), 2.62 (t, 2H, J=7.6 Hz), 1.99 (m, 1H), 1.79 (m, 1H). MS calc 582, found 583 (MH⁺).

Examples 26-42, compounds 24c-24s, Table 3, were prepared according to scheme 2, utilizing either method A or method B.

Alternatively, compounds belonging to this class were prepared according to scheme 2a, as detailed for example 43.

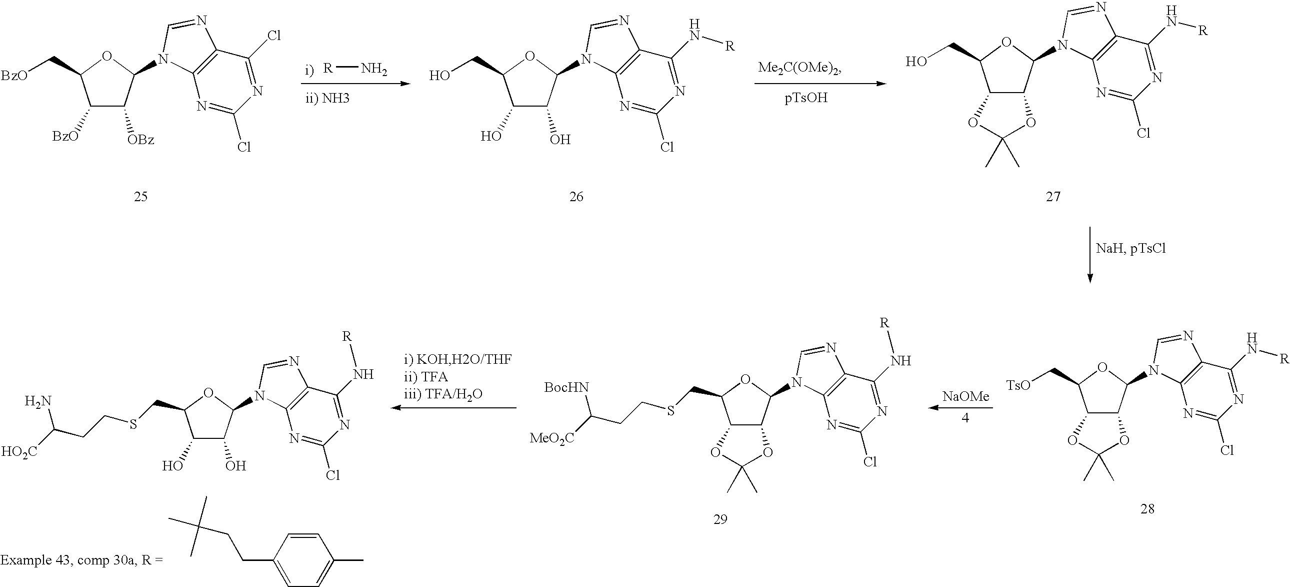

### EXAMPLE 43

### 2-Amino-4-(((2S,3S,4R,5R)-5-(6-(2-(biphenyl-4-yl)ethylamino)-2-chloro-9H-purin-9-yl)-3,4-dihydroxy-tetrahydrofuran-2-yl)methylthio)butanoic acid 30a

### Step 1: (2R,3R.4S,5R)-2-(6-(2-(biphenyl-4-yl)ethylamino)-2-chloro-9H-purin-9-yl)-5(hydroxymethyl)-tetrahydrofuran-3,4-diol 26

A solution of dichloride 25 (863 mg, 1.36 mmol, prepared according to the method of Andrzejewska, Mariola; Kaminski, Jaroslaw; Kazimierczuk, Zygmunt Nucleosides Nucleotides 21, 1, 2002, 73-78) and biphenylethyl amine (803 mg, 4.08 mmol) in DME (8 mL) was stirred for 4 hours at room temperature. The reaction mixture was then diluted in EtOAc (15 mL) and washed with brine (15 mL), dried with Na₂SO₄, filtered and concentrated in vacuo. The crude product was stirred in saturated methanolic solution of ammonia at 70° C. for overnight. The mixture was concentrated in vacuo and purified by flash chromatography using 10% MeOH in DCM to give the title compound in 67% yield (412 mg).

MS: calc 481; found 482 (MH⁺)

### Step 2: ((3aR,4R,6R,6aR)-6-(6-(2-(biphenyl-4-yl)ethylamino)-2-chloro-9H-purin-9-yl)-2,2-dimethyl-tetrahydrofuro[3,4-d][1,3]dioxol-4-yl)methanol 27

A solution of 26 (412 mg, 0.85 mmol), 2,2-dimethoxypropane (530 μL, 446 mg, 4.28 mmol) and pTsOH (180 mg, 0.94 mmol) in acetone (5 mL) was stirred at room temperature for 2 hrs. The reaction was quenched by the addition of Et₃N (200 μL) and concentrated in vacuo. The crude product was purified by flash chromatography using the gradient eluent from 75% to 100% EtOAc in hexanes giving the title compound 27 in 66% yield (292 mg).

MS: calc 521; found 522 (MH⁺)

### Step 3: ((3aR,4R,6R,6aR)-6-(6-(2-(biphenyl-4-yl)ethylamino)-2-chloro-9H-purin-9-yl)-2,2-dimethyl-tetrahydrofuro[3,4-d][1,3]dioxol-4-yl)methyl 4-methylbenzenesulfonate 28

NaH (13 mg 60% mineral oil suspension, 0.32 mmol) was added to a solution of 27 (83 mg, 0.16 mmol) in THF (2 mL) at 0° C. and stirred for 15 min. pTsCl (33 mg, 0.17 mmol) was then added and the reaction mixture was allowed to stir for 1 hour at 0° C. It was diluted in EtOAc (5 mL) and washed sequentially with water (5 mL) and brine (5 mL). The organic layer was dried with Na₂SO₄, filtered and concentrated in vacuo giving the title compound 28 quantitatively (107 mg)

MS: calc 675; found 676 (MH⁺)

### Step 4: Methyl 4-(((3aS,4S,6R,6aR)-6-(6-(2-(biphenyl-4-yl)ethylamino)-2-chloro-9H-purin-9-yl)-2,2-dimethyl-tetrahydrofuro[3,4-d][1,3]dioxol-4-yl)methylthio)-2-(tert-butoxycarbonylamino)butanoate 29

Crude 28 (107 mg, 0.16 mmol) was dissolved in dry MeOH (2 mL) and treated with a solution prepared by mixing Boc thiolactone 4 (51 mg, 0.24 mmol) and NaOMe (480 μL 0.5M solution, 0.24 mmol) for 15 min at room temperature. The resultant reaction mixture was heated at reflux for 4 hours. It was allowed to cool to room temperature and concentrated in vacuo. The residue was diluted in EtOAc (5 mL) and washed with water (5 mL) and brine (5 mL). The organic layer was dried with Na₂SO₄, filtered and concentrated in vacuo. The crude product was purified by flash chromatography eluting with 40%-50% EtOAc in hexanes to give the title compound 29 in 94% yield (87 mg).

MS: calc 752; found 753 (MH⁺)

### Step 5: 2-Amino-4-(((2S,3S,4R,5R)-5-(6-(2-(biphenyl-4-yl)ethylamino)-2-chloro-9H-purin-9-yl)-3,4-dihydroxy-tetrahydrofuran-2-yl)methylthio)butanoic acid 30a

A solution of 29 (87 mg, 0.11 mmol) and KOH (40 mg, 0.71 mmol) in 1:1 mixture of THF and water (3 mL) was stirred at room temperature for 2 hours. The reaction mixture was then concentrated in vacuo and diluted in water (5 mL). It was washed with Et₂O (5 mL) and acidified to pH=2 by addition of citric acid. The aqueous phase was then extracted with EtOAc (3×5 mL) and the combined organic layer was dried with Na₂SO₄, filtered and concentrated in vacuo. The obtained crude product was treated with 2:1 mixture of TFA/water (3 mL) containing trace amount of BHT. The reaction mixture was stirred at room temperature for 45 min and then it was treated with 5 drops of water. It was allowed to stir for another 1 hour before it was concentrated in vacuo. The crude product was purified by flash chromatography using the gradient from 30% MeOH/DCM to 60:30:10 CHCl₃/MeOH/NH₄OH to give the title compound 30a in 32% yield (22 mg). ¹H NMR (DMSO-d₆) δ ppm □8.45 (m, 1H), 8.35 (s, 1H), 7.55 (m, 4H), 7.2-7.4 (m, 5H), 5.79 (d, 1H, J=5.9 Hz), 4.61 (m, 1H), 3.99-4.11 (m, 3H), 3.65 (m, 2H), 2.93 (m, 3H), 2.80 (m, 1H), 2.60 (m, 1H), 2.47 (m, 1H), 1.97 (m, 1H), 1.80 (m, 1H)

MS: calc 598; found 599 (MH⁺)

Compounds belonging to this series, presented in Table 3, were prepared either following Scheme 2, examples 26-42, compounds 24c-s or scheme 2a, example 44-49, compounds 30b-g.

TABLE 3

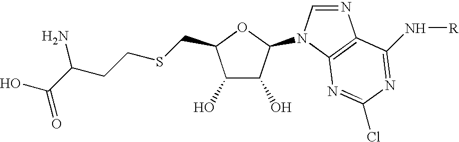

| Ex | Cpd | R/Structure | Name | Characterization | Scheme |
|---|---|---|---|---|---|
| 26 | 24c | 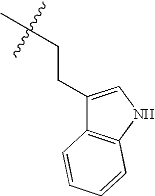 | 4-(((2S,3S,4R,5R)-5-(6-(2-(1H-in-dol-3-yl)ethyl-amino)-2-chlor-o-9H-purin-9-yl)-3,4-di-hydroxy-tetrahydrofuran-2-yl)meth-ylthio)-2-amino-butanoic acid | ¹H NMR(80% CD₃OD, 20% DMSO-d₆,400 MHz): δ 2.06(m, 1H), 2.20(m, 1H),2.76(m, 2H), 2.97(m, 1H), 3.09(m, 1H),3.15(t,2H, J=7.6Hz), 3.63(m, 1H),3.87(t, 2H, J=7.6Hz), 4.22(m, 1H),4.32(m, 1H), 4.76(dd, 1H, J=5.2, 5.2Hz),5.96(d, 1H, J=5.2Hz), 7.06(dd,1H, J=7.6, 7.8Hz), 7.14(dd, 1H, J=7.2,8.0Hz), 7.19(s, 1H), 7.49(d, 1H, J=8.0Hz),7.77(d, 1H, J=8.0Hz), 8.28(s,1H). LCMS(M + H)⁺ calcd 562, found562. | 2 Method A |
| 27 | 24d | 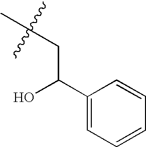 | 2-amino-4-(((2S,3S,4R,5R)-5-(2-chlor-o-6-(2-hy-droxy-2-phenyl-ethylamino)-9H-pur-in-9-yl)-3,4-di-hydroxy-tetra-hydrofuran-2-yl)methyl-thio)butanoicacid | ¹H NMR(DMSO-d₆/D₂O, 400 MHz): δ1.84(m, 1H), 1.98(m, 1H), 2.60(t, 2H,J=7.6Hz), 2.76–2.94(m, 2H),3.30(m,1H), 2H assumed under D₂O, 4.02(m,1H), 4.08–4.14(m, 1H), 4.63(m, 1H),5.80(d, 1H, J=6.0Hz), 7.21–7.43(m,5H), 8.35(s, 1H). LCMS(M + H)⁺ calcd539, found 539. | 2 Method A |
| 28 | 24e | 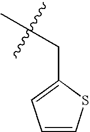 | 2-amino-4-(((2S,3S,4R,5R)-5-(2-chlor-o-6-(thio-phen-2-yl-methylamino)-9H-pur-in-9-yl)-3,4-di-hydroxy-tetrahydrofuran-2-yl)methyl-thio)butanoicacid | ¹H NMR(DMSO-d₆/D₂O, 400 MHz):δ1.75(m, 1H), 1.94(m, 1H), 2.55(t, 2H,J=7.6Hz), 2.69–2.88(m, 2H), 3.22(m,1H),3.96(m, 1H), 4.01–4.08(m, 1H),4.56(dd, 1H, J=5.2, 5.6Hz), 4.70(bs,2H), 5.75(d, 1H, J=5.6Hz), 6.87(m, 1H),6.95(d, 1H, J=3.2Hz), 7.27(dd, 1H,J=0.8, 4.8Hz), 8.33(s, 1H). MS calc514; found 515(MH⁺) | 2 Method A |
| 29 | 24f | 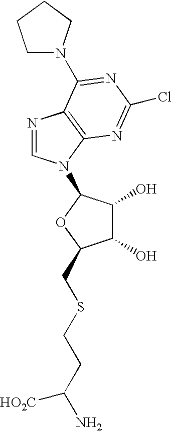 | 2-amino-4-(((2S,3S,4R,5R)-5-(2-chlor-o-6-(pyr-rolidin-1-yl)-9H-pur-in-9-yl)-3,4-dihydroxy-tetrahydrofuran-2-yl)methyl-thio)butanoic acid | NMR(DMSO-d₆/D₂O, 400 MHz);δ 1.76–1.99(m,6H), 2.59(t, 2H, J=7.6Hz),2.77(m, 1H), 2.89(m, 1H), 3.22–3.29(m,1H), 3.56(t, 2H, J=6.8Hz) 4.07(m,3H),4.06–4.12(m, 1H), 4.59(m, 1H),5.80(d, 1H, J=5.6Hz), 8.33(s, 1H).MS calc 472; found 473(MH⁺) | 2 Method A |
| 30 | 24g | 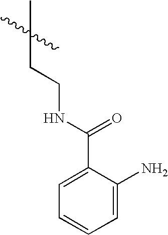 | 2-amino-4-(((2S,3S,4R,5R)-5-(6-(2-(2-amino-benzamido)eth-ylamino)-2-chloro-9H-pur-in-9-yl)-3,4-dihydroxy-tetrahydrofuran-2-yl)methyl-thio)butanoic acid | ¹H NMR(50% DMSO-d₆, 50% CD₃OD,400 MHz): δ 1.89(m, 1H), 2.04(m, 1H,2.61(m, 2H), 2.79–2.93(m,2H), 3.43–3.50(m,3H), 3.63(t, 2H,J=5.6Hz),4.04(m, 1H), 4.13(m, 1H), 4.61(t, 1H,J=5.2Hz), 5.80(d, 1H, J=5.2Hz), 6.46(dd,1H, J=6.8Hz, 7.2Hz), 6.60(dd, 1H,J=1.2, 8.0Hz), 7.06(ddd, 1H, J=1.6, 7.2,8.4Hz), 7.35(d, 1H, J=7.6Hz), 8.18(s,1H). MS calc 580; found 581 (MH⁺). | 2-Method A |
| 31 | 24h | 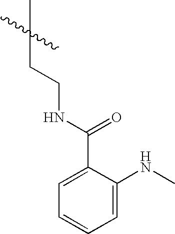 | 2-amino-4-(((2S,3S,4R,5R)-5-(2-chlor-o-6-(2-(2-(methyl-amino)benza-mido)ethylamino)-9H-pur-in-9-yl)-3,4-dihydroxy-tetrahydrofuran-2-yl)methyl-thio)butanoic acid | ¹H NMR(DMSO-d₆/D₂O, 400 MHz): δ1.83(m, 1H), 1.96(m, 1H), 2.54(m, 2H),2.66(s, 3H), 2.74–2.89(m, 2H), 3.34(m,1H), 3.43(m,2H), 3.58(m, 2H), 4.02(m,1H), 4.09(m, 1H), 4.59(t, 1H, J=5.6Hz),5.75(d, 1H, J=6.0Hz), 6.49(m, 1H),6.56(d, 1H, J=8.0Hz), 7.33(m, 1H),7.35(d, 1H, J=7.2Hz), 8.25(s, 1H). MScalc 594, found 595 (MH⁺). | 2 Method A |
| 32 | 24i | 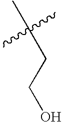 | 2-amino-4-(((2S,3S,4R,5R)-5-(2-chlor-o-6-(2-hydroxy-ethylamino)-9H-pur-in-9-yl)-3,4-dihydroxy-tetrahydrofuran-2-yl)methyl-thio)butanoic acid | ¹H NMR(DMSO-d₆/D₂O, 400 MHz): δ1.89(m, 1H), 2.05(m, 1H), 2.68(t, 2H,J=7.6Hz), 2.82–3.00(m, 2H), 3.29–3.36(m,1H), 3.56(m, 2H), 3.61(m, 2H),4.08(m,1H), 4.07–4.20(m, 1H), 4.69(t, 1H,J=4.8Hz), 5.87(d, 1H, J=6.0Hz), 8.49(s,1H). MS calc 462, found 463(MH⁺). | 2 Method B |
| 33 | 24j | 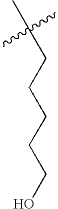 | 2-amino-4-(((2S,3S,4R,5R)-5-(2-chlor-o-6-(5-hydroxy-pentylamino)-9H-pur-in-9-yl)-3,4-dihydroxy-tetrahydrofuran-2-yl)methyl-thio)butanoic acid | ¹H NMR(DMSO-d₆/D₂O, 400 MHz): δ1.31(m, 2H), 1.42(m, 2H), 1.57(m, 2H),1.81(m, 1H), 1.98(m, 1H), 2.60(t, 2H,J=7.6Hz), 2.78(m, 1H), 2.90(m,1H),3.23–3.30(m, 1H), 3.34(m, 2H), 2Hassumed under D₂O,4.00(m, 1H), 4.07–4.13(m,1H), 4.62(dd, 1H, J=5.2,5.6Hz), 5.79(d, 1H, J=5.6Hz), 8.35(s,1H). MS calc 504, found 505(MH⁺) | 2 Method B |
| 34 | 24k | 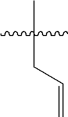 | 4-(((2S,3S,4R,5R)-5-(6-(allyl-amino)-2-chlor-o-9H-purin-9-yl)-3,4-dihydroxy-tetrahydrofuran-2-yl)methyl-thio)-2-amino-butanoic acid | ¹H NMR(DMSO-d₆/D₂O, 400 MHz): δ1.86(m, 1H), 1.99(m, 1H), 2.62(t, 2H,J=7.6Hz), 2.71–2.91(m, 2H), 1Hassumed under D₂O,4.04(m, 3H),4.09–4.13(m,1H), 4.62(m, 1H), 5.06(d, 1H,J=10.4Hz), 5.13(d, 1H, J=17.2Hz),5.81(d, 1H, J=6.0Hz), 5.91(m, 1H),8.38(s, 1H). MS calc 459; found 459(MH⁺) | 2 Method A |
| 35 | 24l | 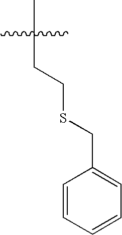 | 2-amino-4-(((2S,3S,4R,5R)-5-(6-(2-(benzyl-thio)ethylami-no)-2-chloro-9H-pur-in-9-yl)-3,4-dihydroxy-tetrahydrofuran-2-yl)methyl-thio)butanoic acid | ¹H NMR(DMSO-d₆/D₂O, 400 MHz): δ1.82(m, 1H), 1.98(m, 1H), 2.60(m, 4H),2.76–2.94(m, 2H), 3.30(m, 1H), 3.61(t,2H, J=7.2Hz), 3.77(s, 2H),4.01(m,1H), 4.08–4.13(m, 1H), 4.63(dd, 1H,J=5.2, 6.0Hz), 5.80(d, 1H, J=6.0Hz),7.17–7.32(m, 5H), 8.36(s, 1H). MS calc568; found 569(MH⁺). | 2 Method B |
| 36 | 24m | 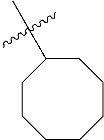 | 2-amino-4-(((2S,3S,4R,5R)-5-(2-chlor-o-6-(cyclo-octylamino)-9H-pur-in-9-yl)-3,4-dihydroxy-tetrahydrofuran-2-yl)methyl-thio)butanoic acid | ¹NMR(DMSO-d₆/D₂O, 400 MHz): δ1.53–2.03(m, 16H), 2.61(t, 2H, J=7.6Hz),2.75–2.94(m, 2H), 3.25–3.43(m,1H), 3.99–4.04(m, 1H),4.07–4.12(m,1H), 4.21(m, 1H), 4.61(m, 1H), 5.79(d,1H, J=6.0Hz), 8.35(s, 1H). MS calcdm/z 528; found 529(MH⁺). | 2 Method A |
| 37 | 24n | 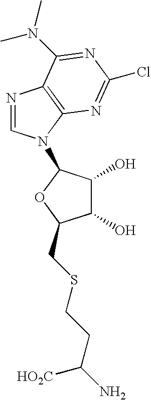 | 2-amino-4-(((2S,3S,4R,5R)-5-(2-chlor-o-6-(di-methylamino)-9H-pur-in-9-yl)-3,4-dihydroxy-tetrahydrofuran-2-yl)methyl-thio)butanoic acid | ¹H NMR(DMSO-d₆/D₂O, 400 MHz): δ1.83(m, 1H), 1.97(m, 1H), 2.59(m, 2H),2.80(m, 1H), 2.89(m, 1H), 3.15(bs,3H), 3.31(m, 1H), 3.61(bs, 3H),4.01(m,1H), 4.10(m, 1H), 4.59(dd, 1H, J=5.2,5.6Hz), 5.80(d, 1H, J=6.0Hz),8.32(s, 1H). MS calc 446; found 447(MH⁺). | 2-Method A |
| 38 | 24o | 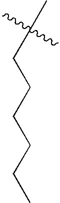 | 2-amino-4-(((2S,3S,4R,5R)-5-(2-chlor-o-6-(hexyl-amino)-9H-pur-in-9-yl)-3,4-dihydroxy-tetrahydrofuran-2-yl)meth-ylthio)butanoic acid | ¹H NMR(DMSO-d₆/D₂O, 400 MHz): δ0.83(m, 3H), 1.26(m, 6H), 1.54(m, 2H),1.83(m, 1H), 1.97(m, 1H), 2.59(m, 2H),2.75–2.96(m, 2H), 3.30(m, 1H),3.39(m,2H), 4.00(m, 1H), 4.09(m, 1H),4.62(dd, 1H, J=5.2, 5.6Hz), 5.78(d, 1H,J=6.4Hz), 8.32(s, 1H). MS calc 502,found 503(MH⁺). | 2 Method B |
| 39 | 24p | 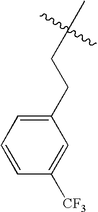 | 2-amino-4-(((2S,3S,4R,5R)-5-(2-chlor-o-6-(3-(tri-fluoromethyl)phenethyl-amino)-9H-pur-in-9-yl)-3,4-dihydroxy-tetrahydrofuran-2-yl)methyl-thio)butanoic acid | ¹H NMR(DMSO-d₆/D₂O, 400 MHz): δ1.82(m, 1H), 1.99(m, 1H), 2.60(t, 2H,J=7.2Hz), 2.74–2.93(m, 2H), 3.00(t,2H,J=6.8Hz), 3.28(m, 1H), 3.69(t, 2H,J=6.8Hz), 4.00(m, 1H), 4.07–4.12(m,1H), 4.61(dd, 1H, J=5.6, 5.6Hz), 5.78(d,1H, J=6.0Hz), 7.50–7.62(m, 4H),8.34(s, 1H). MS calc 590; found 591(MH⁺). | 2 Method B |
| 40 | 24q | 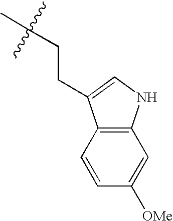 | 2-amino-4-(((2S,3S,4R,5R)-5-(2-chlor-o-6-(2-(6-meth-oxy-1H-indol-3-yl)eth-ylamino)-9H-pur-in-9-yl)-3,4-dihydroxy-tetrahydrofuran-2-yl)meth-ylthio)butanoic acid | ¹H NMR(DMSO-d₆/D₂O, 400 MHz): δ1.83(s, 1H), 1.98(s, 1H), 2.60(t, 2H,J=7.6Hz), 2.78–2.96(m, 4H),3.29(m,1H), 2H presumed under D₂O, 3.72(s,3H), 4.01(m, 1H), 4.10(m, 1H), 4.63(dd,1H, J=5.2, 5.6Hz), 5.80(d, 1H,J=6.0Hz), 6.60(dd, 1H, J=2, 8.4Hz),6.82(d, 1H, J=2.4Hz), 7.00(s, 1H), 7.53(d,1H, J=8.8Hz), 8.33(s, 1H). MS calc591; found 592(MH⁺). | 2 Method B |
| 41 | 24r | 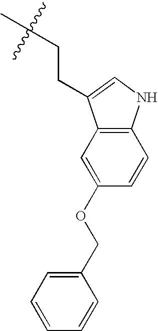 | 2-amino-4-(((2S,3S,4R,5R)-5-(6-(2-(5-(benzyl-oxy)-1H-indol-3-yl)eth-ylamino)-2-chlor-o-9H-purin-9-yl)-3,4-dihydroxy-tetrahydrofuran-2-yl)meth-ylthio)butanoic acid | ¹H NMR(DMSO-d₆/D₂O, 400 MHz): δ1.83(s, 1H), 1.98(s, 1H), 2.60(t, 2H),2.75–2.96(m, 4H), 3.31(m, 1H), 2Hpresumedunder D₂O, 4.02(m, 1H), 4.11(m,1H), 4.63(dd, 1H, J=5.2, 5.6Hz),5.05(s, 2H), 5.80(d, 1H, J=6.0Hz), 6.75(dd,1H, J=2, 8.8Hz), 7.12(s, 1H), 7.21(m,2H), 7.27(m, 1H), 7.34(m, 2H), 7.42(m,2H, J=7.2Hz), 8.34(s, 1H). MS calc667, found 668(MH⁺). | 2 Method B |
| 42 | 24s | 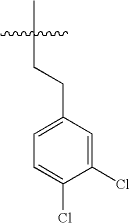 | 2-amino-4-(((2S,3S,4R,5R)-5-(2-chlor-o-6-(3,4-di-chlorophenethyla-mino)-9H-purin-9-yl)-3,4-dihydroxy-tetrahydrofuran-2-yl)methyl-thio)butanoic acid | ¹H NMR(DMSO-d₆/D₂O, 400 MHz): δ1.80(m, 1H), 1.96(m, 1H), 2.60(m, 2H),2.75–2.90(m, 4H), 3.25(m, 1H), 3.65(m,2H), 4.02(m, 1H),4.07–4.12(m, 1H),4.61(dd, 1H, J=5.6Hz, 5.6Hz), 5.79(d,1H, J=6.0Hz), 7.19(m, 1H), 7.47–7.53(m,2H), 8.35(s, 1H). MS calc 590,found 591(MH⁺). | 2 Method B |
| 44 | 30b | 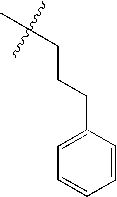 | 2-amino-4-(((2S,3S,4R,5R)-5-(2-chlor-o-6-(3-phenyl-propylamino)-9H-pur-in-9-yl)-3,4-dihydroxy-tetrahydrofuran-2-yl)methyl-thio)butanoic acid | ¹H NMR(DMSO-d₆) δ8.45(s, 1H), 6.48(s,2H), 6.38(s, 1H), 5.89(d, 1H, J=5.3Hz),4.69(t, 1H, J=5.2Hz), 4.0–4.2(m,2H), 3.76(s, 6H), 3.37(m,2H), 2.90(m,3H), 2.70(m, 2H), 2.56(s, 2H), 2.05(m,1H), 1.90(m, 1H)MS: calc 582; found 583(MH⁺) | 2a |
| 45 | 30c | 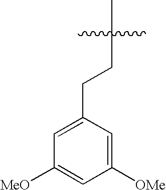 | 2-amino-4-(((2S,3S,4R,5R)-5-(2-chlor-o-6-(3,5-di-methoxyphenethylamino)-9H-purin-9-yl)-3,4-dihydroxy-tetrahydrofuran-2-yl)meth-ylthio)butanoic acid | ¹H NMR(DMSO-d₆) δ8.45(s, 1H), 6.48(s,2H), 6.38(s, 1H), 5.89(d, 1H, J=5.3Hz),4.69(t, 1H, J=5.2Hz), 4.0–4.2(m,2H), 3.76(s, 6H),3.37(m, 2H), 2.90(m,3H), 2.70(m, 2H), 2.56(s, 2H), 2.05(m,1H), 1.90(m, 1H)MS: calc 582; found 583(MH⁺) | 2a |
| 46 | 30d | 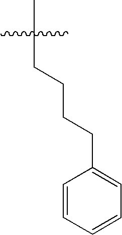 | 2-amino-4-(((2S,3S,4R,5R)-5-(2-chlor-o-6-(4-phenyl-butylamino)-9H-pur-in-9-yl)-3,4-dihydroxy-tetrahydrofuran-2-yl)methyl-thio)butanoic acid | ¹H NMR(CD₃OD) δ8.17(s, 1H), 7.1–7.3(m,5H), 5.91(d, 1H, J=4.5Hz), 4.75(, 1H),4.33(m, 1H), 4.20(m, 1H), 4.07(m,1H), 3.57(m, 2H), 3.01(m, 2H),2.74(m,2H), 2.66(m, 2H), 2.23(m, 1H), 2.08(m,1H)MS: calc 550; found 551(MH⁺) | 2a |
| 47 | 30e | 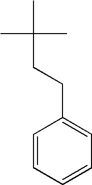 | 2-amino-4-(((2S,3S,4R,5R)-5-(2-chlor-o-6-(phenethyl-amino)-9H-pur-in-9-yl)-3,4-dihydroxy-tetrahydrofuran-2-yl)methyl-thio)butanoic acid | ¹H NMR(DMSO-d₆) δ8.45(s,1H), 7.2–7.4(m,5H), 5.89(d, 1H, J=4.7Hz), 4.69(m,1H), 4.0–4.3(m, 2H), 3.71(m, 1H),3.38(m, 1H), 2.8–3.1(m, 4H),2.68(m,2H), 2.56(m, 1H), 2.05(m, 1H), 1.91(m,1H)MS: calc 522; found)523(MH⁺) | 2a |
| 48 | 30f | 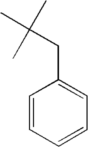 | 2-amino-4-(((2S,3S,4R,5R)-5-(6-(benzyl-amino)-2-chlor-o-9H-purin-9-yl)-3,4-dihydroxy-tetrahydrofuran-2-yl)methyl-thio)butanoic acid | ¹H-NMR: DMSO-d₆ δ(ppm) 9.02(br,1H), 8.45(s, 1H), 7.4–7.2(m, 5H), 5.85(d,J=5.5Hz, 1H), 4.65(m, 2H), 4.15–3.95(m,2H), 3.35(m, 1H), 2.91(m,1H),2.83(m, 1H), 2.65(m, 2H), 1.99(m, 1H),1.87(m, 1H)MS: calc. 508.13(100%; 510.13(38%);found 509.1(100%), 511.1(40%) (MH⁺) | |
| 49 | 30g | 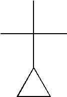 | 2-amino-4-(((2S,3S,4R,5R)-5-(2-chlor-o-6-(cyclo-propylamino)-9H-pur-in-9-yl)-3,4-dihydroxy-tetrahydrofuran-2-yl)methyl-thio)butanoic acid | ¹H-NMR: DMSO-d₆ δ(ppm) 8.52(br,1H), 8.41(s, 1H), 5.83(d, J=5.3Hz, 1H),4.61(m, 1H), 4.13(m, 1H), 4.04(m, 1H),3.37(m, 1H), 2.91(m, 1H),2.81(m,1H), 2.64(m, 2H), 2.51(m, 1H), 1.98(m,1H), 1.88(m, 1H), 0.75(m, 2H), 0.64(m, 2H)MS: calc. 458.11(100%), 460.11(37%);found 459.2(100%), 461.2(27%) (MH⁺) | |

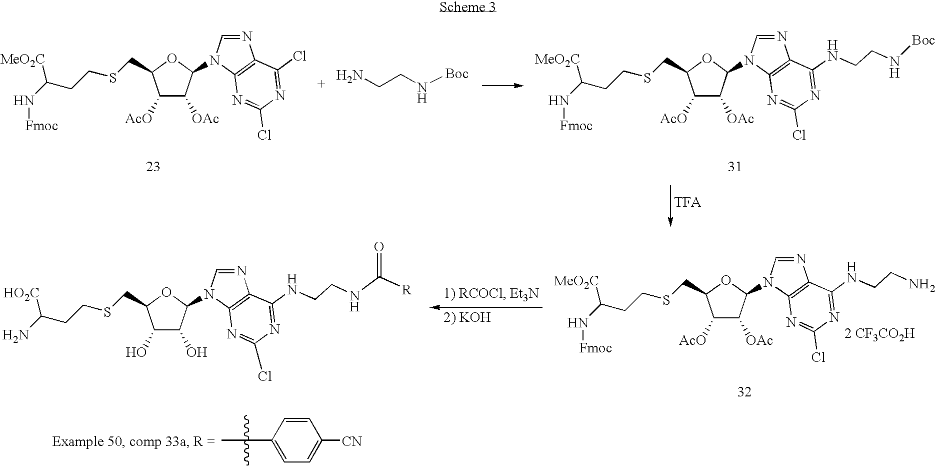

### EXAMPLE 50

### 2-amino-4-(((2S,3S,4R,5R)-5-(2-chloro-6-(2-(4-cyanobenzamido)ethylamino)-9H-purin-9-yl)-3,4-dihydroxy-tetrahydrofuran-2-yl)methylthio)butanoic acid 33a

### Step 1: (2S,3S,4R,5R)-2-((3-(((9H-fluoren-9-yl)methoxy)carbonylamino)-4-methoxy-4-oxobutylthio)methyl)-5-(6-(2-(tert-butoxycarbonylamino)ethylamino)-2-chloro-9H-purin-9-yl)-tetrahydrofuran-3,4-diyl diacetate 31

A solution of 23 (500 mg, 0.659 mmol), N-boc-ethylenediamine (211 mg, 1.32 mmol) and triethylamine (275 ml, 1.98 mmol) in ethanol (5 ml) and 1,2-dichloroethane (5 ml) was stirred overnight at room temperature. The reaction was then diluted with CH₂Cl₂ (50 ml) and washed with 5% HCl(aq) (2×25 ml), saturated NaHCO₃ (25 ml) and brine (25 ml). The organic phase was then dried with Na₂SO₄, filtered and evaporated to give the crude product. The crude product was purified by flash chromatography using 80% CH₂Cl₂ and 20% acetone to give the title compound 31 in 74.8% yield (435 mg) as white solid. MS: calc. 882.4, found 882.3 (MH⁺).

### Step 2: (2S,3S,4R,5R)-2-((3-(((9H-fluoren-9-yl)methoxy)carbonylamino)-4-methoxy-4-oxobutylthio)methyl)-5-(6-(2-aminoethylamino)-2-chloro-9H-purin-9-yl)-tetrahydrofuran-3,4-diyl diacetate 32

To a solution of 31 (300 mg, 0.340 mmol) in 1.0 ml CH₂Cl₂ was added trifluoroacetic acid (1.0 ml). The reaction was stirred for 1 hour and the solvent was evaporated. The residue was re-dissolved in methylene chloride and the solvent was removed (twice) to give the title compound 32 in a quantitative yield as the di-trifluoroacetic acid salt.

### Step 3: 2-amino-4-(((2S,3S,4R,5R)-5-(2-chloro-6-(2-(4-cyanobenzamido)ethylamino)-9H-purin-9-yl)-3,4-dihydroxy-tetrahydrofuran-2-yl)methylthio)butanoic acid 33a

A solution of 32 (76 mg, 0.075 mmol), triethylamine (62.6 μl, 0.45 mmol) and 4-cyanobenzoyl chloride (18.5 mg, 0.112 mmol) in 0.75-1.0 ml THF was stirred for 16 hours at room temperature. 1.5 ml of 1N KOH was then added and the reaction was stirred for 3 hours. The reaction was then quenched with formic acid (100 μl) and the solvent was evaporated. The crude product was purified by flash chromatography using 65% CHCl₃, 30% methanol and 5% NH₄OH then triturated with CH₃CN. The title compound 33a was obtained in 15% yield as white solid (10 mg). ¹H NMR (DMSO-d₆/D₂O): δ 1.83 (m, 1H), 1.97 (m, 1H), 2.60 (t, 2H, J=7.6 Hz), 2.77-289 (m, 2H), 3.27 (m, 1H), 2H presumed under D₂O, 3.65 (m, 2H), 4.01 (m, 1H), 4.08-4.13 (m, 1H), 4.62 (dd, 1H, J=4.8, 5.6 Hz), 5.79 (d, 1H, J=5.6 Hz), 7.89-7.95 (m, 4H), 8.35 (s, 1H).

MS: calc 590, found 591 ((MH⁺).

Examples 51-61 compounds 33b-33l, Table 4, were prepared from compound 32 and the appropriate acid chloride, scheme 3, as described for example 50, step 3.

TABLE 4

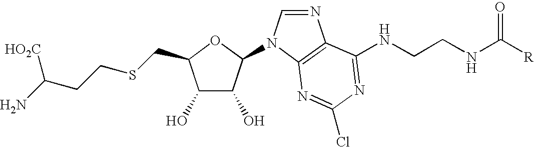

| Ex | Cpd | R/Structure | Name | Characterization | Scheme |
|---|---|---|---|---|---|
| 51 | 33b | 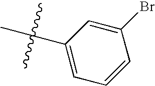 | 2-amino-4-(((2S,3S,4R,5R)-5-(6-(2-(3-bromo-benzamido)eth-ylamino)-2-chloro-9H-pur-in-9-yl)-3,4-dihydroxy-tetrahydrofuran-2-yl)meth-ylthio)butanoic acid | ¹H NMR(DMSO-d₆/D₂O): δ 1.81(m,1H), 1.98(m, 1H), 2.61(m, 2H),2.77–2.91(m, 2H), 3.25(m, 1H),3.50(m, 2H), 3.60(m, 2H),4.01(m,1H), 4.08–4.14(m, 1H), 4.63(dd,1H, J=5.2, 5.2Hz), 5.80(d, 1H,J=5.6Hz), 7.40(m, 1H), 7.69(d, 1H,J=7.6Hz), 7.79(d, 1H, J=7.2Hz),7.97(s, 1h), 8.37(s, 1H), 8.70(bs,1H). MS calc 645; found 646(MH⁺). | 3 |
| 52 | 33c | 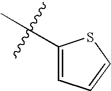 | 2-amino-4-(((2S,3S,4R,5R)-5-(2-chlor-o-6-(2-(thio-phene-2-carbox-amido)ethylami-no)-9H-purin-9-yl)-3,4-dihydroxy-tetrahydrofuran-2-yl)methyl-thio)butanoic acid | ¹H NMR(DMSO-d₆/D₂O): δ1.81(m,1H), 1.99(m, 1H), 2.61(m, 2H),2.75–2.94(m, 2H), 3.29(m, 1H), 2Hpresumedunder D₂O, 3.58(m, 2H),4.00(m, 1H), 4.08–4.13(m, 1H),4.63(m, 1H), 5.80(d, 1H, J=6.0Hz),7.10(dd, 1H, J=3.6, 4.8Hz),7.70(m, 2H), 8.37(s, 1H). MS calc571; found 572(MH⁺). | 3 |
| 53 | 33d | 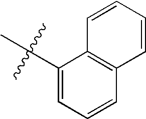 | 4-(((2S,3S,4R,5R)-5-(6-(2-(1-naph-thamido)ethylami-no)-2-chloro-9H-pur-in-9-yl)-3,4-dihydroxy-tetrahydrofuran-2-yl)meth-ylthio)-2-aminobutanoic acid | ¹H NMR(DMSO-d₆/D₂O): δ 1.81(m,1H), 1.97(m, 1H), 2.60(m, 2H),2.75–2.93(m, 2H), 3.26(m, 1H), 2Hassumed under D₂O,3.69(m, 2H),4.01(m, 1H), 4.07–4.13(m, 1H),4.63(dd, 1H, J=4.8, 6.0Hz), 5.80(d,1H, J=6.0Hz), 7.41–7.55(m,3H), 7.56(d, 1H, J=6.8Hz), 7.91(d,1H, J=8.0Hz), 7.95(d, 1H,J=8.0Hz), 8.07(d, 1H, J=8.8Hz),8.36(s, 1H), 8.62(t, 1H, J=5.6Hz).MS calc 615, found 616(MH⁺). | 3 |
| 54 | 33e | 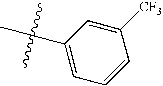 | 2-amino-4-(((2S,3S,4R,5R)-5-(2-chlor-o-6-(2-(3-(tri-fluoromethyl)benza-mido)ethylamino)-9H-pur-in-9-yl)-3,4-dihydroxy-tetrahydrofuran-2-yl)methyl-thio)butanoic acid | ¹H NMR(DMSO-d₆/D₂O): δ 1.81(m,1H), 1.99(m, 1H), 2.60(m, 2H),2.75–2.91(m, 2H), 3.26(m, 1H),2Hassumed under D₂O, 3.62(m, 2H),4.04(m, 1H), 4.07–4.13(m, 1H),4.62(dd, 1H, J=5.2, 5.6Hz), 5.79(d,1H, J=5.6Hz), 7.68(dd, 1H,J=8.0, 8.0Hz), 7.86(d, 1H,J=7.2Hz), 8.08(d, 1H, J=8.0Hz),8.12(s, 1H), 8.35(s, 1H). MS calc633; found 634(MH⁺) | 3 |
| 55 | 33f | 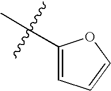 | 2-amino-4-(((2S,3S,4R,5R)-5-(2-chlor-o-6-(2-(furan-2-carbox-amido)ethylami-no)-9H-purin-9-yl)-3,4-di-hydroxy-tetra-hydrofuran-2-yl)meth-ylthio)butanoic acid | ¹H NMR(DMSO-d₆/D₂O): δ 1.83(m,1H), 1.98(m, 1H), 2.61(m, 2H),2.76–2.92(m, 2H), 3.31(m, 1H),3.47(m,2H), 2H under D₂O, 4.03(m,1H), 4.08–4.13(m, 1H), 4.62(dd,1H, J=5.2, 5.2Hz), 5.80(d, 1H,J=6.0Hz), 6.58(m, 1H), 7.05(d,1H, J=2.8Hz), 7.76(s, 1H), 8.21(s,1H), 8.36(s, 1H). MS calc 555;found 556(MH⁺) | 3 |
| 56 | 33g | 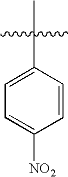 | 2-amino-4-(((2S,3S,4R,5R)-5-(2-chlor-o-6-(2-(4-nitro-benzamido)ethylamino)-9H-purin-9-yl)-3,4-dihydroxy-tetrahydrofuran-2-yl)meth-ylthio)butanoic acid | ¹H NMR(DMSO-d₆/D₂O): δ 1.81(m,1H), 1.97(m, 1H), 2.60(m, 2H),2.75–2.93(m, 2H), 3.28(m, 1H), 2Hpresumed under D₂O,3.63(m, 2H),4.01(m, 1H), 4.07–4.13(m, 1H),4.62(dd, 1H, J=4.8, 6.0Hz), 5.80(d,1H, J=5.6Hz), 8.02(d, 2H,J=8.8Hz), 8.16(s, 1H), 8.26(d, 2H,J=8.8Hz), 8.35(s, 1H). MS calc610; found 611(MH⁺) | 3 |
| 57 | 33h | 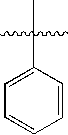 | 2-amino-4-(((2S,3S,4R,5R)-5-(6-(2-benz-amidoethylamino)-2-chlor-o-9H-purin-9-yl)-3,4-dihydroxy-tetrahydrofuran-2-yl)meth-ylthio)butanoic acid | ¹H NMR(DMSO-d₆/D₂O): δ 1.83(m,1H), 1.99(m ,1H), 2.60(m, 2H),2.79–2.93(m, 2H), 3.28(m, 1H),3.61(m, 2H), 2H presumed underD₂O,4.01(m, 1H), 4.09–4.13(m,1H), 4.62(dd, 1H, J=5.2, 5.6Hz),5.80(d, 1H, J=6.0Hz), 7.40–7.50(m,3H), 7.79(m, 2H), 8.19(s, 1H),8.35(s, 1H), 8.57(m, 1H). MS calc565; found 566(MH⁺) | 3 |
| 58 | 33i | 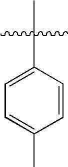 | 2-amino-4-(((2S,3S,4R,5R)-5-(2-chlor-o-6-(2-(4-methyl-benzamido)eth-ylamino)-9H-purin-9-yl)-3,4-dihydroxy-tetrahydrofuran-2-yl)meth-ylthio)butanoic acid | ¹H NMR(DMSO-d₆/D₂O): δ 1.80(m,1H), 1.97(m, 1H), 2.33(s, 3H),2.60(m, 2H), 2.76–2.90(m, 2H),3.24(m, 1H), 3.49(m, 2H),3.60(m,2H), 4.01(m, 1H), 4.07–4.13(m,1H), 4.62(dd, 1H, J=5.2, 5.6Hz),5.80(d, 1H, J=6.0Hz), 7.22(d, 2H,J=8.0Hz), 7.70(d, 2H, J=8.0Hz),8.60(s, 1H). ). MS calc: 579;found 580(MH⁺) | 3 |
| 59 | 33j | 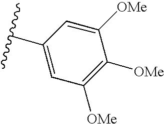 | 2-amino-4-(((2S,3S,4R,5R)-5-(2-chlor-o-6-(2-(3,4,5-tri-methoxybenzamido)eth-ylamino)-9H-pur-in-9-yl)-3,4-dihydroxy-tetrahydrofuran-2-yl)meth-ylthio)butanoic acid | ¹H NMR(DMSO-d₆/D₂O): δ 1.82(m,1H), 1.98(m ,1H), 2.61(m, 2H),2.75–2.91(m, 2H), 3.25(m, 1H),3.49(m,2H), 3.60(m, 2H), 3.68(s,3H), 3.80(s, 6H), 4.01(m, 1H),4.08–4.14(m, 1H), 4.63(dd, 1H,J=5.2, 5.6Hz), 5.80(d, 1H,J=5.6Hz), 7.13(s, 2H), 8.37(s, 1H),8.56(m, 1H). MS calc 655; found656(MH⁺) | 3 |
| 60 | 33k | 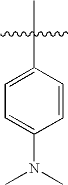 | 2-amino-4-(((2S,3S,4R,5R)-5-(2-chlor-o-6-(2-(4-(dimethyl-amino)benzamido)eth-ylamino)-9H-purin-9-yl)-3,4-dihydroxy-tetrahydrofuran-2-yl)methyl-thio)butanoic acid | ¹H NMR(DMSO-d₆/D₂O): δ 1.81(m,1H), 1.98(m, 1H), 2.61(m, 2H),2.75–2.92(m, 2H), 2.94(s, 6H),3.26(m, 1H), 2Hassumed underD₂O, 3.58(m, 2H), 4.03(m, 1H),4.08–4.13(m, 1H), 4.63(dd, 1H,J=5.2, 5.6Hz), 5.80(d, 1H,J=6.0Hz), 6.66(d, 2H, J=8.8Hz),7.67(d, 2H, J=8.4Hz), 8.23(m,1H), 8.36(s, 1H). MS calc 608;found 609(MH)⁺ | 3 |
| 61 | 33l | 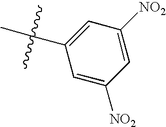 | 2-amino-4-(((2S,3S,4R,5R)-5-(2-chlor-o-6-(2-(3,5-dinitro-benzamido)ethylamino)-9H-pur-in-9-yl)-3,4-dihydroxy-tetrahydrofuran-2-yl)methyl-thio)butanoic acid | ¹H NMR(DMSO-d₆/D₂O): δ 1.81(m,1H), 1.96(m, 1H), 2.59(m, 2H),2.79–2.91(m, 2H), 3.26(m, 1H), 2Hassumed underD₂O, 3.65(m, 2H),4.00(m, 1H), 4.08–4.13(m, 1H),4.61(m, 1H), 5.78(d, 1H, J=6.4Hz),8.35(s, 1H), 8.92(m, 1H), 8.97(d,2H, J=1.6Hz). MS calc 655; found656(MH)⁺ | 3 |

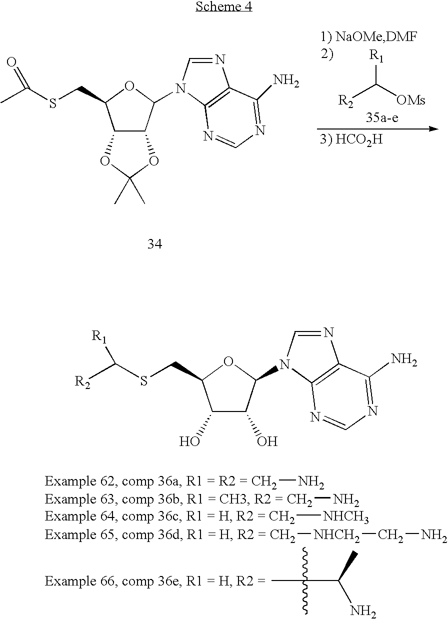

### General Procedure for the Synthesis of the BOC-Protected Amino Alcohols 35a-e

A solution of the appropriate amino alcohol (6.0 mmol), di-tert-butyl dicarbonate (1.96 g, 9.0 mmol) and NaHCO₃ (2.5 g, 30.0 mmol) in 10 ml of dioxane and 10 ml H₂O was stirred overnight at room temperature. Ethyl acetate (50 ml) was then added and the organic phase was washed with saturated NaHCO₃ (2×50 ml), 10% HCl (2×50 ml), and brine (50 ml). The organic phase was then dried with MgSO₄, filtered and evaporated. The product was purified as indicated.

### Di-tert-butyl 2-hydroxypropane-1,3-diyldicarbamate 35a

The title compound 35a was prepared in 35% yield (610 mg) using the general procedure and employing 3.92 g of di-tert-butyl dicarbonate instead of the indicated amount and the product was obtained as white solid after recrystalized from hexanes.

### (R)-Tert-butyl 2-hydroxypropylcarbamate 35b

The title compound 35b was obtained in 69.5% yield (730 mg) as white solid after flash chromatography using 50% hexanes and 50% ethyl acetate.

### Tert-butyl 2-hydroxyethyl(methyl)carbamate 35c

The title compound 35c was obtained in 32.4% yield (340 mg) as white solid after flash chromatography using 50% hexanes and 50% ethyl acetate.

### Tert-butyl 2-tert-butyloxycarbonyl-amidoethyl(2-hydroxyethyl)carbamate 35d

The title compound 35d was prepared using the general procedure with the following modification. 3.92 g of di-tert-butyl dicarbonate was used instead of the indicated amount. Purification by flash chromatography using 50% hexanes and 50% ethyl acetate gave the desired in 18.6% yield (340 mg) as white solid.

### (R)-Tert-butyl 1-hydroxypropan-2-ylcarbamate 35e

The title compound 35e was obtained in 72.4% yield (760 mg) as white solid after purification by flash chromatography using 50% hexanes and 50% ethyl acetate.

### EXAMPLE 62

### (2R,3R,4S,5S)-2-(6-amino-9H-purin-9-yl)-5-((1,3-diaminopropan-2-ylthio)methyl)-tetrahydrofuran-3,4-diol 36a

Methane sulfonyl chloride (103 mg, 0.9 mmol) was added to a solution of di-tert-butyl 2-hydroxypropane-1,3-diyldicarbamate 35a (174.2 mg, 0.6 mmol) in 1.0 ml pyridine. The reaction was mixed and left to stand for 30 minutes. Methylene chloride (10 ml) was then added and the reaction was washed with H₂O (3×10 ml). The organic phase was dried with MgSO₄, filtered and evaporated to give the crude mesylate.

A solution of 34 (73 mg, 0.200 mmol, Baddiley and Jamieson, J. Chem. Soc. 1955, 1085; Guillerm et al. J. Med Chem. 2001, 44, 2743Pignot et al. Eur. J. Org. Chem 2000, 549) and NaOMe (400 μl, 0.20 mmol, 0.5 M in methanol) in DMF (1.0 ml) was stirred for 10 minutes under an N₂ atmosphere. Then, the mesylate of 35a from above, dissolved in 1.0 ml DMF, was added and the reaction was stirred overnight at room temperature under an N₂ atmosphere. The reaction was diluted with ethyl acetate (10 ml) and washed with H₂O (3×10 ml). The organic phase was evaporated and 1.0 ml of 50% TFA in dichloromethane was added. After 1 h the solvent was evaporated and the title compound 36a was purified by prep-HPLC (compound could be isolated as di-TFA salt). ¹H NMR (DMSO-d₆) δ (ppm): 8.32 (s, 1H), 8.13 (s, 1H), 7.23 (s, 2H), 5.88 (d, 1H, J=5.6 H-z), 4.73 (dd, 1H, J=4.8, 5.2 Hz), 4.15 (dd, 1H, J=4.0, 4.4 Hz), 4.05 (m, 1H), 3.09 (m, 3H), 2.87-3.03 (m, 4H). MS: calc 355.4; found 356 (MH)⁺.

Examples 63-66, Table 5, were prepared in a manner similar to example 62, scheme 3, using compounds 35b-e in place of 35a The compounds were isolated as the formate salts after prep-HPLC purification in 20.6%; 33.8%; 18.9; and 47% yields respectively.

TABLE 5

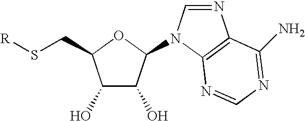

| Ex. | Cpd | R/Structure | Name | Characterization | Scheme |
|---|---|---|---|---|---|
| 63 | 36b | 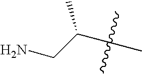 | (2R,3R,4S,5S)-2-(6-ami-no-9H-purin-9-yl)-5-(((S)-1-amino-propan-2-yl-thio)methyl)-tetra-hydrofuran-3,4-diol | ¹H NMR(DMSO-d₆) δ (ppm): 8.33(s,1H), 8.29(s, 1H), 8.12(s, 1H),7.28(s, 2H), 5.87(d, 1H, J=5.6Hz),5.87(d, 1H, J=5.6Hz), 4.73(dd,1H, J=5.2, 5.6Hz), 4.14(m,1H),4.00(m, 1H), 3.17(m, 1H), 2.84–2.98(m,2H), 2.57–2.70(m,2H), 1.11(d, 3H, J=6.4Hz). MS calc340.4, found 341(MH)⁺ | 4 |
| 64 | 36c | 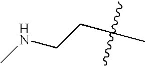 | (2R,3R,4S,5S)-2-(6-ami-no-9H-pur-in-9-yl)-5-((2-(meth-ylamino)eth-ylthio)methyl)-tetra-hydrofuran-3,4-diol | ¹H NMR(DMSO-d₆) δ (ppm): 8.33(s,1H), 8.27(s, 1H), 8.12(s, 1H),7.28(bs, 2H), 5.87(d, 1H, J=6.0Hz),4.73(dd, 1H, J=5.2, 5.2Hz),4.13(dd, 1H,J=4.0, 4.4Hz), 3.99(m,1H), 2.82–2.95(m, 2H), 2.77(t,2H, J=6.8Hz), 2.63–2.67(m, 2H),2.31(s, 3H). MS calc 340.4, found341(MH)⁺ | 4 |
| 65 | 36d | 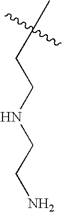 | (2R,3R,4S,5S)-2-(6-ami-no-9H-pur-in-9-yl)-5-((2-(2-amino-ethylami-no)ethylthio)meth-yl)-tetrahydrofuran-3,4-diol | ¹H NMR(CD₃OD) δ (ppm): 8.34(s,2H), 8.30(s, 1H), 8.20(s, 1H), 5.99(d,1H, J=4.4Hz), 4.80(dd, 1H,J=4.8, 5.2Hz), 4.35(dd, 1H, J=5.2,5.2Hz),4.21(m, 1H), 2.97–3.04(m,4H), 2.87–2.91(m, 4H), 2.74–2.78(m,2H). MS calc 369.4, found370(MH)⁺ | 4 |
| 66 | 36e | 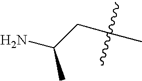 | (2R,3R,4S,5S)-2-(6-ami-no-9H-pur-in-9-yl)-5-(((R)-2-amino-propylthio)meth-yl)-tetrahydrofuran-3,4-diol | ¹H NMR(DMSO-d₆) δ (ppm): 8.33(s,1H), 8.31(s, 1H), 8.12(s, 1H),7.28(s, 2H), 5.86(d, 1H, J=5.6Hz),4.74(dd, 1H, J=4.8, 5.6Hz),4.13(dd, 1H, J=4.0, 4.8Hz),4.01(m,1H), 3.12(m, 1H), 2.90(m,2H), 2.59(d, 2H, J=6.8Hz),1.08(d, 3H, J=6.8Hz). MS calc 340.4;found 341(MH)⁺ | 4 |

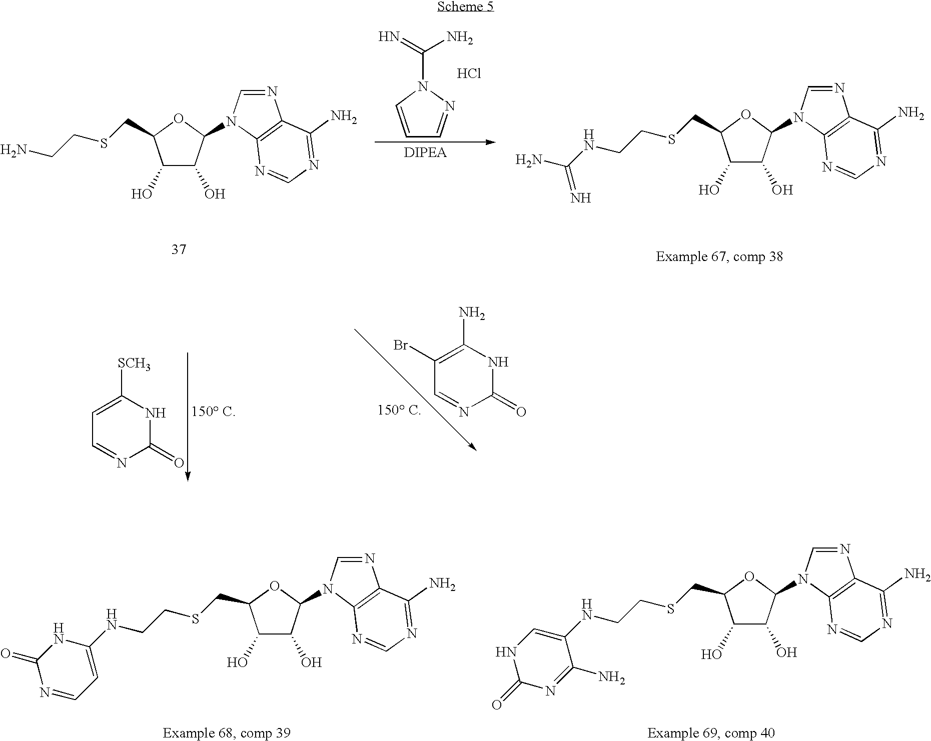

### EXAMPLE 67

### 1-(2-(((2S,3S,4R,5R)-5-(6-amino-9H-purin-9-yl)-3,4-dihydroxy-tetrahydrofuran-2-yl)methylthio)ethyl)guanidine 38

A solution of (2R,3R,4S,5S)-2-(6-amino-9H-purin-9-yl)-5-((2-aminoethylthio)methyl)-tetrahydrofuran-3,4-diol 37 (65 mg, 0.2 mmol, Jamieson, G. A., J. Org. Chem. 1963, 28, 2397-2400), 1H-pyrazole-1-carboxamidine hydrochloride (32 mg, 0.22 mmol) and DIPEA (77 μl, 0.44 mmol) in dry DMF (1.0 ml) was stirred at room temperature for 4 hours. The solvent was then evaporated and the product was purified by preparative HPLC to give the title compound 38 as the formate salt in 48.8% yield (36 mg). ¹H NMR (DMSO-d₆/D₂O) δ (ppm): 8.45 (s, 1H), 8.34 (s, 1H), 8.29 (s, 1H), 8.11 (s, 1H), 5.84 (d, 1H, J=6.0 Hz), 4.69 (dd, 1H, J=5.6, 5.6 Hz), 4.11 (m, 1H), 3.99 (m, 1H), 3.23 (m, 2H), 2.82-2.94 (m, 2H), 2.61 (m, 2H). MS calc 368.4; found 369 (MH)⁺.

### EXAMPLE 68

### 6-(2-(((2S,3S,4R,5R)-5-(6-amino-9H-purin-9-yl)-3,4-dihydroxy-tetrahydrofuran-2-yl)methylthio)ethylamino)pyrimidin-2(1H)-one 39

A solid mixture of 34 (100 mg, 0.306 mmol) and 4-methylthiouracil (14.5 mg, 0.102 mmol) was heated at 150° C. for 15 minutes according to the method of Delia, T. J. et al (J. Org. Chem. 1965, 30, 2766-2768). After the reaction had cooled to room temperature, the product was purified by preparative HPLC to give the title compound 39 in 28% yield (12 mg). ¹H NMR (DMSO-d₆/D₂O) δ (ppm): 8.46 (s, 1H), 8.28 (s, 1H), 7.69 (d, 1H, J=7.6 Hz), 5.91 (d, 1H, J=7.6 Hz), 5.89 (d, 1H, J=6.0 Hz), 4.68 (dd, 1H, J=5.2, 5.6 Hz), 4.12 (m, 1H), 4.03 (m, 1H), 2H assumed under D₂O, 2.93 (m, 2H), 2.71 (m, 2H). MS: calc 420.45; found 421 (MH)⁺

### EXAMPLE 69

### 4-amino-5-(2-(((2S,3S,4R,5R)-5-(6-amino-9H-purin-9-yl)-3,4-dihydroxy-tetrahydrofuran-2-yl)methylthio)ethylamino)pyrimidin-2(1H)-one 40

A solid mixture of 34 (100 mg, 0.306 mmol) and 5-bromocytosine (58 mg, 0.305 mmol) was heated at 150° C. for 4 hours. After the reaction had cooled to room temperature, the product was purified by preparative HPLC to give the title compound 40 as the formate salt in 8.4% yield (11 mg). ¹H NMR (DMSO-d₆/D₂O) δ (ppm): 8.31 (s, 1H), 8.15 (s, 1H), 8.11 (s, 1H), 6.62 (s, 1H), 5.85 (d, 1H, J=5.6 Hz), 4.70 (dd, 1H, J=5.2, 5.6 Hz), 4.13 (m, 1H), 4.00 (m, 1H), 2.90 (m, 4H), 2.65 (m, 2H). MS calc 435.46; found 436 (MH)⁺.

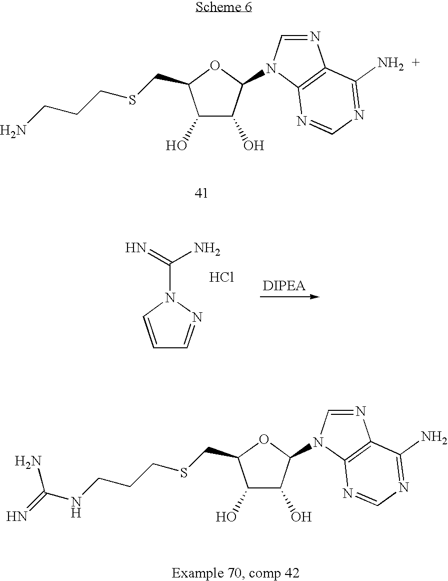

### EXAMPLE 70

### 1-(3-(((2S,3S,4R,5R)-5-(6-amino-9H-purin-9-yl)-3,4-dihydroxy-tetrahydrofuran-2-yl)methylthio)propyl)guanidine 42

The title compound 42, was prepared in 57.5% yield (44 mg) as the formate salt according to the procedure described in example 67 starting from (2R,3R,4S,5S)-2-(6-amino-9H-purin-9-yl)-5-((3-aminopropylthio)methyl)-tetrahydrofuran-3,4-diol 41 (63 mg, 0.2 mmol, prepared according to the method of Borchardt, R. T., and Wu, Y. S. J. Med. Chem. 1974, 17, 862-7). ¹H NMR (CD₃OD) δ (ppm): 8.43 (s, 1H), 8.29 (s, 1H), 8.19 (s, 1H), 5.99 (d, 1H, J=4.8 Hz), 4.81 (dd, 1H, J=4.8, 5.2 Hz), 4.34 (dd, 1H, J=4.8, 5.2 Hz), 4.20 (m, 1H), 3.23 (t, 2H, J=6.8 Hz), 2.97 (m, 2H), 2.63 (t, 2H, J=6.8 Hz), 1.82 (m, 2H). MS calc 382.4; found 383 (MH)⁺

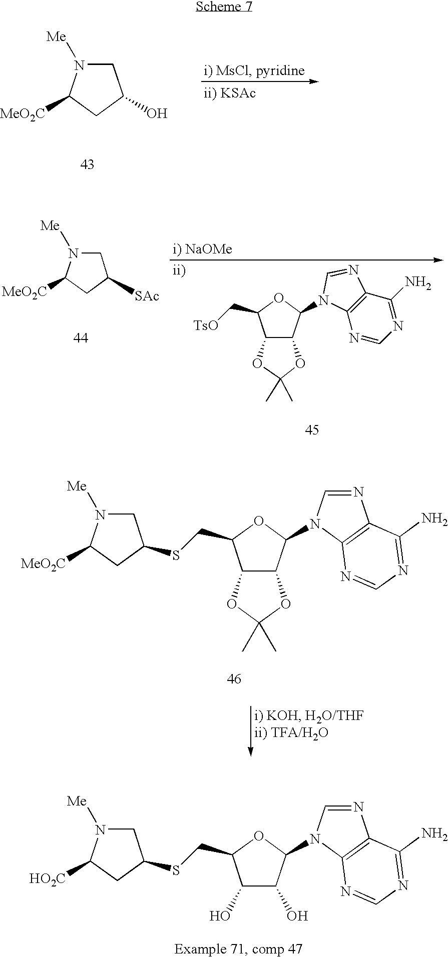

### EXAMPLE 71

### (2S,4S)-4-(((2S,3S,4R,5R)-5-(6-amino-9H-purin-9-yl)-3,4-dihydroxy-tetrahydrofuran-2-yl)methylthio)-1-methylpyrrolidine-2-carboxylic acid 47

### Step 1: (2S,4S)-methyl 4-(acetylthio)-1-methylpyrrolidine-2-carboxylate 44

Mesyl chloride (170 μL, 251 mg, 2.25 mmol) was added to a solution of (2S,4R)-methyl 4-hydroxy-1-methylpyrrolidine-2-carboxylate 43 (239 mg, 1.5 mmol, prepared according to the procedure of R. L. Elliott et al., Synthesis, 1995, 772) in pyridine (3 mL) and stirred at room temperature overnight. The reaction mixture was diluted in DCM (20 mL) and washed with water (15 mL). The organic phase was separated, dried with Na₂SO₄, filtered and concentrated in vacuo. Thecrude mesylate was diluted in DMF and treated with KSAc (800 mg, 7.5 mmol). The reaction mixture was stirred for 2 hours at 80° C. and then it was concentrated in vacuo. The title compound 44 was obtained in 29% yield (95 mg) after flash chromatography using 30% EtOAC in hexane.

MS: calc 217; found 240 (M+Na⁺)

### Step 2: (2S,4S)-methyl 4-(((3aS,4S,6R,6aR)-6-(6-amino-9H-purin-9-yl)-2,2-dimethyl-tetrahydrofuro[3,4-d[[1,3]dioxol-4-yl)methylthio)-1-methylpyrrolidine-2-carboxylate 46

A solution of thioacetate 44 (95 mg, 0.43 mmol) in MeOH (2 mL) was treated with 0.5 M solution of NaOMe (876 μL). The reaction mixture was stirred for 15 minutes at room temperature and then it was transferred to a solution of ((3aR,4R,6R,6aR)-6-(6-amino-9H-purin-9-yl)-2,2-dimethyl-tetrahydrofuro[3,4-d][1,3]dioxol-4-yl)methyl 4-methylbenzenesulfonate 45 (184 mg, 0.4 mmol, Thompson et al., J. Org. Chem. 1999, 64,(20), 7467-73) in MeOH (2 mL). The combined mixture was further refluxed for 2 hours. After it was allowed to cool down, it was concentrated in vacuo. The crude material was diluted in EtOAc (10 mL) and water. The layers were separated and the organic phase was washed with brine, dried with Na₂SO₄, filtered and concentrated in vacuo. The title compound 46 was obtained in 24% yield (45 mg) after flash chromatography using 5% MeOH in DCM.

MS: calc 464; found 465 (MH⁺)

### Step 3: (2S,4S)-4-(((2S,3S,4R,5R)-5-(6-amino-9H-purin-9-yl)-3,4-dihydroxy-tetrahydrofuran-2-yl)methylthio)-1-methylpyrrolidine-2-carboxylic 47

A solution of 46 (24 mg, 0.05 mmol) and KOH (40 mg, 0.71 mmol) in 1:1 mixture of THF and water (3 mL) was stirred at room temperature for 2 hours. The reaction mixture was then concentrated in vacuo and diluted in water (5 mL). It was washed with Et₂O (5 ml) and acidified to pH=2 by addition of citric acid. The aqueous phase was then extracted with EtOAc (3×5 mL) and the combined organic layer was dried with Na₂SO₄, filtered and concentrated in vacuo. The residue was treated with 2:1 mixture of TFA/water (3 mL) containing trace amount of BHT. The reaction mixture was stirred at room temperature for 45 min and then it was treated with 5 drops of water. It was allowed to stir for another 1 hour before it was concentrated in vacuo. The title compound 47 was obtained in 44% yield (9 mg) after flash chromatography eluting with 30% MeOH/DCM to 60:30:10 CHCl₃/MeOH/NH₄OH. ¹H NMR (D₂O) δppm); 8.12 (s, 1H), 8.02 (s, 1H), 5.86 (d, 1H, J=4.9 Hz), 4.70 (t, 1H, J=5.1 Hz), 4.26 (t, 1H, J=5.2 Hz), 4.17 (dd, 1H, J=4.9 Hz, J=11.2 Hz), 3.72 (dd, 1H, J=6.9 Hz, J=9.5 Hz), 3.54 (m, 1H), 3.39 (dd, 1H, J=4.0 Hz, J=11.6 Hz), 3.13 (dd, 1H, J=6.7 Hz, J=12.1 Hz); 2.92 (dd, 1H, J=4.5 Hz, J=14.3 Hz), 2.83 (dd, J=6.2 Hz, J=14.3 Hz), 2.69 (s, 3H), 1.92 (m, 1H)

MS: calc 410; found 411 (MH⁺)

Examples 72-76, compounds 48a-48e, Table 6, were prepared in a manner similar to example 71, scheme 7 with the following modifications

Example 72, compound 48a, was prepared in a manner similar to example 71, were step 1, was conducted according to the procedure in J. Org. Chem. 1196, 61, 2226, replacing (2S,4R)-methyl 4-hydroxy-1-methylpyrrolidine-2-carboxylate 43, with (2S,4S)-1-tert-butyl 2-methyl 4-hydroxypiperidine-1,2-dicarboxylate.

Example 73, compound 48b, was prepared in a manner similar to example 71, scheme 7. Step 1 was carried out according to the procedure for example 72 replacing (2S,4R)-methyl 4-hydroxy-1-methylpyrrolidine-2-carboxylate 43, with (2S,4R)-1-tert-butyl 2-methyl 4-hydroxypiperidine-1,2-dicarboxylate.

Example 74, compound 48c, was prepared in a manner similar to example 71. Step 1 was conducted according to the procedure in J. Med. Chem. 2001, 44, 94, replacing (2S,4R)-methyl 4-hydroxy-1-methylpyrrolidine-2-carboxylate 43, with tert-butyl 3-hydroxyazetidine-1-carboxylate.

Example 75, compound 48d, was prepared in a manner similar to example 71, replacing (2S,4R)-methyl 4-hydroxy-1-methylpyrrolidine-2-carboxylate 43, with (R)-tert-butyl 2-(hydroxymethyl)pyrrolidine-1-carboxylate.

Example 76, compound 48e, was prepared in a manner similar to example 71, replacing (2S,4R)-methyl 4-hydroxy-1-methylpyrrolidine-2-carboxylate 43, with (S)-tert-butyl 2-(hydroxymethyl)pyrrolidine-1-carboxylate.

TABLE 6

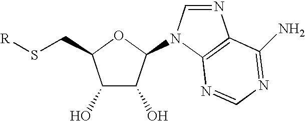

| Ex. | Cpd | R/Structure | Name | Characterization | Scheme |
|---|---|---|---|---|---|
| 72 | 48a | 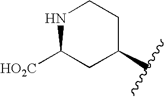 | (2S,4R)-4-(((2S,3S,4R,5R)-5-(6-ami-no-9H-purin-9-yl)-3,4-dihydroxy-tetrahydrofuran-2-yl)meth-ylthio)piperidine-2-car-boxylic acid | ¹H NMR(D₂O) δ8.17(s, 1H), 8.07(s,1H), 5.91(br.s, 1H), 4.32(,1H), 4.21(m, 1H), 3.78(m, 1H),2.9–3.1(m, 4H), 2.02(m, 2H),1.81(m, 1H),1.60(m, 1H), 1.56(m,1H)MS: calc 410; found 411(MH⁺) | 7 |
| 73 | 48b | 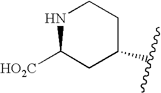 | (2S,4S)-4-(((2S,3S,4R,5R)-5-(6-ami-no-9H-purin-9-yl)-3,4-dihydroxy-tetrahydrofuran-2-yl)meth-ylthio)piperidine-2-car-boxylic acid | ¹H NMR(D₂O) δ8.28(m, 1H),8.17(m, 1H), 6.02(d, 1H, J=4.9Hz),4.4(m, 1H), 4.37(m, 1H),3.84(m, 1H), 2.8–3.4(m, 5H),2.05(m, 2H), 1.86(m,1H), 1.50(m,1H)MS: calc 410; found 411(MH⁺) | 7 |
| 74 | 48c | 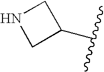 | (2R,3R,4S,5S)-2-(6-ami-no-9H-purin-9-yl)-5-((aze-tidin-3-yl-thio)methyl)-tetra-hydrofuran-3,4-diol | ¹H NMR(CH₃OH-d3) δ8.27(s,1H), 8.22(s, 1H), 5.97(d, 1H,J=4.7Hz), 4.81(t, 1H, J=5.1Hz),4.31(t, 1H, J=5.1Hz), 4.16(m,3H), 4.01(m, 1H), 3.88(dd,1H,J=6.8Hz, J=10.6Hz), 3.79(dd,1H, J=7.0Hz, J=10.8Hz), 3.02(m,2H)MS: calc 338; found 339(MH⁺) | 7 |
| 75 | 48d | 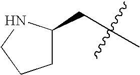 | (2R,3R,4S,5S)-2-(6-ami-no-9H-purin-9-yl)-5-(((R)-pyr-rolidin-2-yl-methylthio)methyl)-tetra-hydrofuran-3,4-diol | ¹H NMR(CH₃OH-d3) δ8.28(s,1H), 8.19(s, 1H), 5.99(d, 1H,J=4.6Hz), 4.84(t, 1H, J=5.1Hz),4.36(t, 1H, J=5.3Hz), 4.20(m,1H), 3.70(m,1H), 2.9–3.2(m,5H), 2.77(dd, 1H, J=9.2Hz,J=14.3Hz), 2.05(m, 2H), 1.91(m,1H), 1.61(m, 1H)MS: calc 366; found 367(MH⁺) | 7 |
| 76 | 48e | 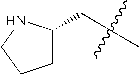 | (2R,3R,4S,5S)-2-(6-ami-no-9H-purin-9-yl)-5-(((S)-pyr-rolidin-2-yl-methylthio)methyl)-tetra-hydrofuran-3,4-diol | ¹H NMR(CH₃OH-d3) δ8.27(s,1H), 8.20(s, 1H), 5.99(d, 1H,J=4.9Hz), (1H assumed underH₂O at 4.87), 4.36(t, 1H,J=5.1Hz), 4.24(m, 1H), 3.68(m,1H),3.24(m, 2H), 3.0–3.2(m, 3H),2.73(dd, 1H, J=9.4Hz, 14.1Hz),1.9–2.1(m, 3H), 1.57(m, 1H)MS: calc 366; found 367(MH⁺) | 7 |

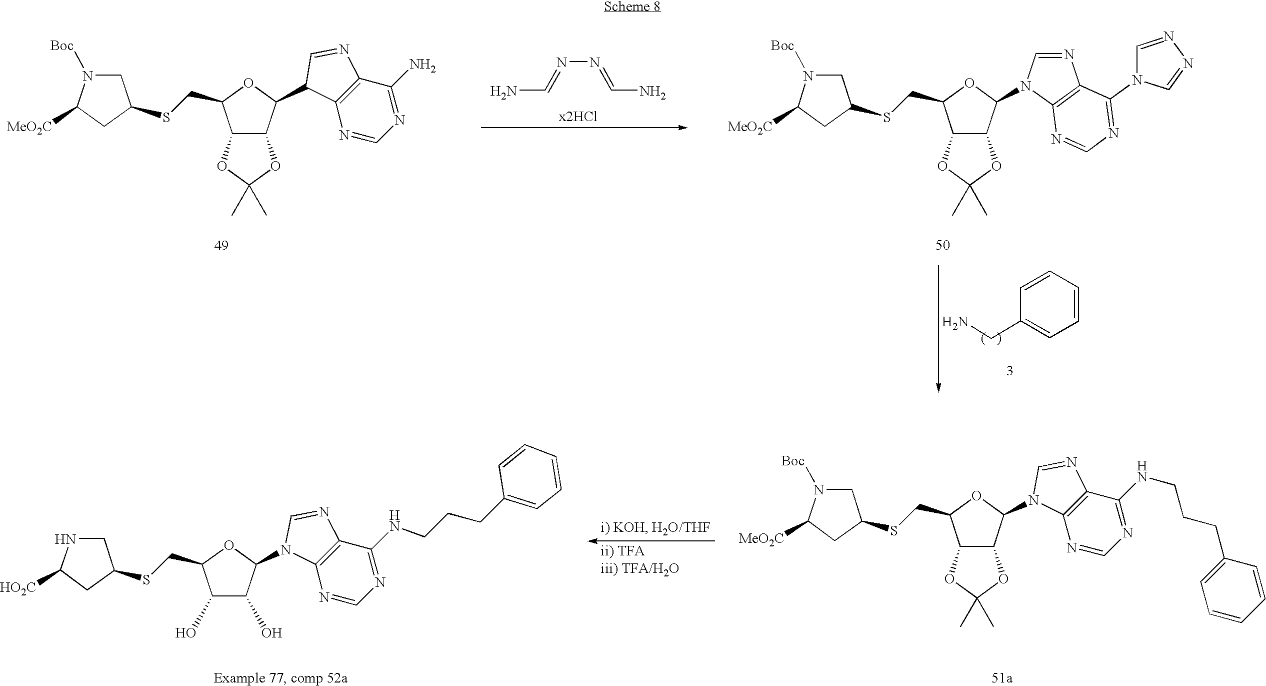

### EXAMPLE 77

### (2S,4S)-4-(((2S,3S,4R,5R)-3,4-dihydroxy-5-(6-(3-phenylpropylamino)-9H-purin-9-yl)-tetrahydrofuran-2-yl)methylthio)pyrrolidine-2-carboxylic acid 52a

### Step 1: (2S,4S)-1-tert-butyl 2-methyl 4-(((3aS,4S,6R,6aR)-6-(6-(4H-1,2,4-triazol-4-yl)-9H-purin-9-yl)-2,2-dimethyl-tetrahydrofuro[3,4-d][1,3]dioxol-4-yl)methylthio)pyrrolidine-1,2-dicarboxylate 50

Azine dihydrochloride (603 mg, 2.82 mmol) was added to a solution of (2S,4S)-1-tert-butyl 2-methyl 4-(((3aS,4S,6R,6aR)-6-(6-amino-9H-purin-9-yl)-2,2-dimethyl-tetrahydrofuro[3,4-d][1,3]dioxol-4-yl)methylthio)pyrrolidine-1,2-dicarboxylate 49 (310 mg, 0.56 mmol, prepared as described by J. Q. Guo et al, Bioorg Med. Chem. Let., 1993, 3 (2), 147-152) in dry pyridine (3 mL) and the reaction mixture was refluxed overnight. It was then cooled to room temperature and concentrated in vacuo. The crude product was purified by flash chromatography using EtOAc giving the title compound 50 in 32% yield (108 mg).

MS: calc 602; found 603 (MH⁺)

### Step 2: (2S,4S)-1-tert-butyl 2-methyl 4-(((3aS,4S,6R,6aR)-2,2-dimethyl-6-(6-(3-phenylpropylamino)-9H-purin-9-yl)-tetrahydrofuro[3,4-d][1,3]dioxol-4-yl)methylthio)pyrrolidine-1,2-dicarboxylate 51a

A solution of 50 (45 mg, 0.085 mmol, 1 equiv.) and 3-phenylpropan-1-amine (77 μL, 72 mg, 0.54 mmol) in dry DMF (1 mL) was stirred at room temperature for 2 days. The reaction mixture was concentrated, diluted in EtOAc (10 mL) and washed with brine (10 mL). The organic phase was dried with Na₂SO₄, filtered and concentrated. The crude product was purified by flash chromatography using EtOAc in hexanes giving the title compound 51 a in 37% yield (44 mg).

MS: calc 668; found 669 (MH⁺)

### Step 3: (2S,4S)-4-(((2S,3S,4R,5R)-3,4-dihydroxy-5-(6-(3-phenylpropylamino)-9H-purin-9-yl)-tetrahydrofuran-2-yl)methylthio)pyrrolidine-2-carboxylic acid 52a

A solution of 51a (44 mg, 0.06 mmol) and KOH (40 mg, 0.71 mmol) in 1:1 mixture of THF and water (3 mL) was stirred at room temperature for 3 hours. The reaction mixture was then concentrated in vacuo and diluted in water (5 mL). It was washed with Et₂O (5 mL) and acidified to pH=2 by addition of citric acid. The aqueous phase was then extracted with EtOAc (3×5 mL) and the combined organic layer was dried with Na₂SO₄, filtered and concentrated in vacuo. The crude product was treated with 2:1 mixture of DCM/TFA (3 mL) containing trace amount of BHT. The reaction mixture was stirred at room temperature for 45 min and then it was treated with 5 drops of water. It was allowed to stir for another 1 hour before it was concentrated in vacuo. The crude product was purified by flash chromatography using the gradient from 30% MeOH/DCM to 60:30:10 CHCl₃/MeOH/NH₄OH to give the title compound 52a in 47% yield (14 mg). ¹H NMR (DMSO-d₆) δ8.32 (s, 1H), 8.19 (s, 2H), 7.89 (br.s, 1H), 7.1-7.3 (m, 5H), 5.87 (d, 1H, J=5.5 Hz), 4.70 (m, 1H), 4.13 (m, 1H), 4.02 (m, 1H), 3.60 (m, 2H), 2.8-3.0 (m, 5H), 2.63 (m, 2H), 2.47 (m, 1H), 1.91 (m, 2H), 1.70 (m, 2H).

MS: calc 514; found 515 (MH⁺).

Examples 78-83, compounds 52b-52g, Table 7, were prepared according to the descriptions below:

Example 78, compound 52b, was prepared according to the method shown in scheme 2a, example 43, replacing 2-(biphenyl-4-yl)ethanamine with 3-phenylpropan-1-amine in step 1, and utilizing (2S,4S)-1-tert-butyl 2-methyl 4-(acetylthio)pyrrolidine-1,2-dicarboxylate in place of tert-butyl 2-oxo-tetrahydrothiophen-3-ylcarbamate in step 4.

Example 79, 52c, was prepared as shown in scheme 1a, example 1, in step 3 replacing tert-butyl 2-oxo-tetrahydrothiophen-3-ylcarbamate with (2S,4S)-1-tert-butyl 2-methyl 4-(acetylthio)pyrrolidine-1,2-dicarboxylate.

Example 80, 52d, was prepared according to scheme 2a, example 43, step 4 reacting 28 with (2S,4S)-1-tert-butyl 2-methyl 4-(acetylthio)pyrrolidine-1,2-dicarboxylate in place of tert-butyl 2-oxo-tetrahydrothiophen-3-ylcarbamate.

Example 81, 52e, was prepared as shown in scheme 1a, example 1, in step 3 replacing tert-butyl 2-oxo-tetrahydrothiophen-3-ylcarbamate with (S)-tert-butyl 3-(acetylthio)pyrrolidine-1-carboxylate.

Example 82, 52f, was prepared according to scheme 2a, example 43, step 4 reacting 28 with (S)-tert-butyl 3-(acetylthio)pyrrolidine-1-carboxylate in place of tert-butyl 2-oxo-tetrahydrothiophen-3-ylcarbamate.

Example 83, 52 g, was prepared according to the method shown in scheme 2a, example 43, replacing 2-(biphenyl-4-yl)ethanamine with 3-phenylpropan-1-amine in step 1, and utilizing (S)-tert-butyl 3-(acetylthio)pyrrolidine-1-carboxylate in place of tert-butyl 2-oxo-tetrahydrothiophen-3-ylcarbamate in step 4.

TABLE 7

| Ex. | Cpd | R/Structure | Name | Characterization | Scheme |
|---|---|---|---|---|---|
| 78 | 52b | 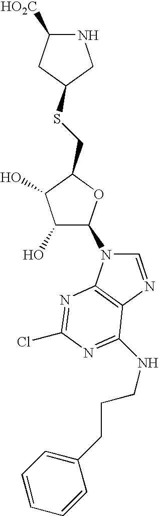 | (2S,4S)-4-(((2S,3S,4R,5R)-5-(2-chlor-o-6-(3-phenyl-propylamino)-9H-pur-in-9-yl)-3,4-dihydroxy-tetrahydrofuran-2-yl)meth-ylthio)pyrrolidine-2-car-boxylic acid | ¹H NMR(CH₃OH-d3) δ8.21(s,1H), 7.2–7.5(m, 5H), 6.04(d,1H, J=4.3Hz), 4.80(m,1H),4.47(m, 1H), 4.37(m, 1H),4.08(m, 1H), 3.73(m, 2H),3.63(m, 1H), 3.46(m, 2H),3.40(m, 1H), 3.21(m, 2H),2.86(m, 3H), 2.1–2.3(m, 2H),MS: calc 548; found 549(MH⁺) | 8 |
| 79 | 52c | 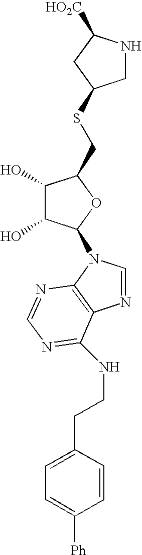 | (2S,4S)-4-(((2S,3S,4R,5R)-5-(6-(2-(bi-phenyl-4-yl)ethyl-amino)-9H-pur-in-9-yl)-3,4-dihydroxy-tetrahydrofuran-2-yl)meth-ylthio)pyrrolidine-2-car-boxylic acid | ¹H NMR(DMSO-d₆) δ8.26(s,1H), 8.18(s, 2H), 7.86(br.s,1H), 7.54(m, 4H), 7.35(m,2H),7.24(m, 3H), 5.82(d, 1H,J=5.5Hz), 4.64(m, 1H), 4.06(m,1H), 3.94(m, 1H), 3.65(m,3H), (3H assumed under H₂Oat 4.87), 2.8–3.0(m, 5H), 1.68(m,1H)MS: calc 576; found 577(MH⁺) | 8 |
| 80 | 52d | 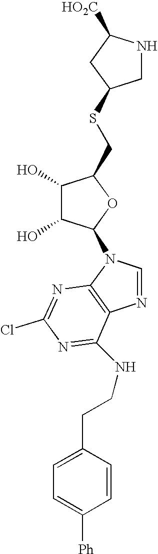 | (2S,4S)-4-(((2S,3S,4R,5R)-5-(6-(2-(bi-phenyl-4-yl)ethyl-amino)-2-chlor-o-9H-purin-9-yl)-3,4-dihydroxy-tetrahydrofuran-2-yl)meth-ylthio)pyrrolidine-2-car-boxylic acid | ¹H NMR(DMSO-d₆) δ8.49(s,1H), 7.6–7.7(m, 4H), 7.1–7.5(5H),5.85(d, 1H, 5.9Hz),4.66(m,1H), 4.13(m, 1H), 4.04(m,1H), 3.3–3.8(m, 7H), 2.98(m,4H), 1.76(m, 1H)MS: calc 610; found 611(MH⁺) | 8 |
| 81 | 52e | 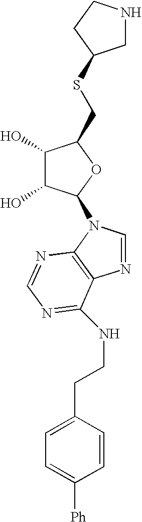 | (2R,3R,4S,5S)-2-(6-(2-(bi-phenyl-4-yl)ethyl-amino)-9H-pur-in-9-yl)-5-(((S)-pyr-rolidin-3-yl-thio)methyl)-tetra-hydrofuran-3,4-diol | ¹H NMR(CH₃OH-d3) δ8.27(br.s,1H), 7.5–7.6(m, 4H), 7.2–7.5(m,5H), 5.99(m, 1H, J=4.7Hz),4.78(t, 1H,J=5.1Hz),4.34(t, 1H, J=5.1Hz), 4.22(m,1H), 3.87(br.s, 1H), (2Hassumed under H₂O at 3.33),2.9–3.1(m, 6H), 2.83(m, 1H),2.70(dd, 1H, J=5.2Hz, J=12.0Hz),2.15(m, 1H), 1.63(m, 1H)MS: calc 532; found 533(MH⁺) | 8 |
| 82 | 52f | 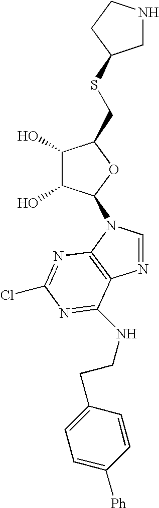 | (2R,3R,4S,5S)-2-(6-(2-(bi-phenyl-4-yl)eth-ylamino)-2-chlor-o-9H-purin-9-yl)-5-(((S)-pyr-rolidin-3-yl-thio)methyl)-tetra-hydrofuran-3,4-diol | ¹H NMR(CH₃OH-d3) δ8.13(s,1H), 7.53(m, 4H), 7.38(m,2H), 7.28(m, 3H), 5.90(d, 1H,J=4.7Hz),4.77(m, 1H), 4.35(m,1H), 4.21(m, 1H), 3.81(m,1H), 3.68(m, 1H), 3.1–3.5(m,5H), 3.08(m, 2H), 2.99(, 2H),2.31(m, 1H), 1.94(m, 1H)MS: calc 566; found 567(MH⁺) | 8 |
| 83 | 52g | 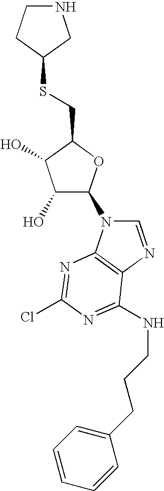 | (2R,3R,4S,5S)-2-(2-chlor-o-6-(3-phenyl-propylamino)-9H-pur-in-9-yl)-5-(((S)-pyr-rolidin-3-yl-thio)methyl)-tetra-hydrofuran-3,4-diol | ¹H NMR(CH₃OH-d3) δ8.14(s,1H), 7.1–7.3(m, 5H), 5.91(d,1H, J=4.7Hz), 4.80(m, 1H),4.36(m, 1H),4.22(m, 1H),3.70(m, 1H), 3.2–3.6(m, 6H),3.09(m, 2H), 2.71(t, 2H, J=7.5Hz),2.34(m, 1H), 1.95(m, 3H)MS: calc 504; found 505(MH⁺) | 8 |

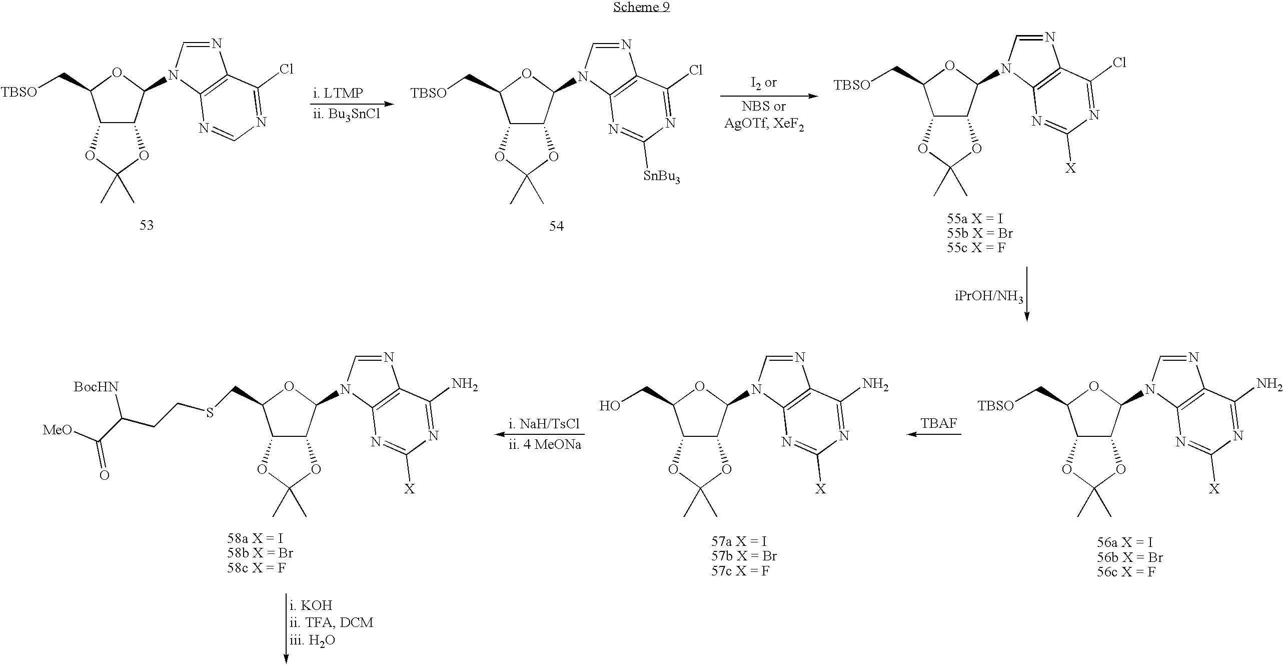

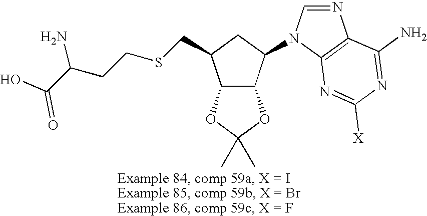

Synthesis of the common intermediate, compound 54 used for Examples 84-86

### 9-((3aR,4R,6R,6aR)-6-((tert-butyldimethylsilyloxy)methyl)-2,2-dimethyl-tetrahydrofuro[3,4-d][1,3]dioxol-4-yl)-6-chloro-2-(tributylstannyl)-9H-purine 54

The reaction is a modification of the procedures reported procedures (K. Kato et al Tet Let 1995, 36, 6507-10; and K. Kato et al, J. Org. Chem 1997, 62, 6833-41). n-BuLi (2.5 M in hexanes, 15 mL) was added to a solution of 2,2,6,6-tetramethylpiperidine (141.26 mL, 0.837 mmol) in THF (76 mL) at 0° C. and the reaction mixture stirred 30 min. The solution was cooled down to −78° C. and a solution of 53 (3.37 g, 7.64 mmol, prepared according to the method of Camp, David; Li, Ying; McCluskey, Adam; Moni, Roger W.; Quinn, Ronald J; Biorg. Med. Chem. Lett. 1998, 8 (6), 695-698) in THF (50 mL) was added drop wise maintaining the temperature bellow −70° C. The mixture stirred 30 min at −78° C. Bu₃SnCl (10.3 mL, 38.2 mmol) was added and the reaction mixture stirred 1 h at −78° C. The mixture was quenched with NH₄Cl (aq. sat. solution), warmed up at room temperature and concentrated under reduced pressure. The residue dissolved in EtOAc, washed with water and brine, dried (Na₂SO₄) and concentrated. The residue was purified by flash chromatography (EtOAc/Hex 1/5) to give the title compoundin 82% yield as clear syrup (4.59 g). ¹H-NMR CDCl₃ δ (ppm): 7.90 (s, 1H), 6.15 (d, J=2.3 Hz, 1H), 5.59 (br, 1H), 5.0 (dd, J=2.9 Hz, J=6.3 Hz, 1H), 4.36 (m, 1H), 3.82 (dd, J=4.5 Hz, J=11.2 Hz, 1H), 3.72 (dd, J=5.9 Hz, J=11.2 Hz, 1H), 1.61 (s, 9H), 1.41 (s, 3H), 1.37 (m, 6H), 1.27 (m, 6H), 0.91 (t, J=7.2 Hz, 1H), 0.85 (s, 9H), −0.016 (s, 3H) −0.024 (s, 3H).

MS calc 730.27, found 730.5 (MH⁺).

### EXAMPLE 84

### 2-amino-4-(((2S,3S,4R,5R)-5-(6-amino-2-iodo-9H-purin-9-yl)-3,4-dihydroxy-tetrahydrofuran-2-yl)methylthio)butanoic acid 59a

### Step 1: 9-((3aR,4R,6R,6aR)-6-((tert-butyldimethylsilyloxy)methyl)-2,2-dimethyl-tetrahydrofuro[3,4-d][1,3]dioxol-4-yl)-6-chloro-2-iodo-9H-purine 55a

Iodine was added in one portion to a solution of 54 (1.5 gr. 2.04 mmol) in THF (20 mL), and the solution was stirred for 1 h at room temperature. The crude was diluted with EtOAc, washed with aqueous NaₐSₛO₃, brine, dried (Na₂SO₄) and concentrated. The crude was purified by flash chromatography (EtOAc/Hex 1/5) giving the title compound 55a in 95% yield (1.1 g) as a pale yellow solid.

¹H-NMR: CDCl₃ δ (ppm) 8.27 (s, 1H), 6.16 (d, J=2.2 Hz, 1H), 5.09 (dd, J=2.2, J=5.7 Hz, 1H), 4.91 (dd, J=2.4 Hz, J=5.7 Hz, 1H), 4.41 (m, 1H), 3.89 (dd, J=3.1 Hz, J=11.4 Hz, 1H), 3.79 (dd, J=3.5 Hz, J=3.8 Hz, 1H), 1.63 (s, 3H), 1.40 (s, 3H), 0.88 (s, 9H), 0.07 (s, 3H). MS: calc 566.06 (100%), 568.06 (38%); found 567.1 (100%), 569.1 (38%) (MH⁺)

### Step 2: 9-((3aR,4R,6R,6aR)-6-((tert-butyldimethylsilyloxy)methyl)-2,2-dimethyl-tetrahydrofuro[3,4-d][1,3]dioxol-4-yl)-2-iodo-9H-purin-6-amine 56a

Ammonia gas was bubbled at 0° C. in a suspension of 55a (1.1 gr, 1.94 mmol) in i-PrOH. The mixture was stirred over night in a sealed tube at 100° C. The mixture was cooled down to 0° C., the sealed tube opened and the system slowly warmed up to room temperature. The solvent was removed under reduced pressure and the residue purified by flash chromatography EtOAc/Hex 1:1 giving the title compound 56a in 79% yield (871 mg) as white solid. ¹H-NMR: CDCl₃ δ (ppm) 7.92 (s, 1H), 6.12 (d, J=2.2 Hz, 1H), 6.06 (s, 2K), 5.19 (dd, J=2.2 Hz, J=6.2 Hz, 1H), 4.96 (dd, J=3.1 Hz, J=4.9 Hz, 1H), 4.36 (m, 1H), 3.89 (dd, J=3.9 Hz, J=11.4 Hz, 1H), 3.80 (dd, J=4.8 Hz, J=11.4 Hz, 1H), 1.63 (s, 3H), 1.40(s, 3H), 0.88 (s, 9H), 0.05 (s, 3H). MS: calc 547.46; found 548.3 (MH⁺)

### Step 3: ((3aR,4R,6R,6aR)-6-(6-amino-2-iodo-9H-purin-9-yl)-2,2-dimethyl-tetrahydrofuro[3,4-d][1,3]dioxol-4-yl)methanol 57a

TBAF was added to a solution of 56a (250 mg, 0.46 mmol) in THF at −25° C. The solution was allowed to warm up to room temperature. The crude was concentrated, and the residue purified by flash chromatography (MeOH/DCM 20:1) giving the title compound 57a in 80% yield (158.6 mg) as light yellow solid. ¹H-NMR: CDCl₃ δ (ppm): 7.75 (s, 1H), 6.77 (br, 1H), 5.82 (d, J=4.7 Hz, 1H), 5.2 (dd, J=4.7 Hz, J=5.9 Hz, 1H), 5.09 (dd, J=1 Hz, J=5.9 Hz, 1H), 4.51 (m, 1H), 3.98 (dd, J=1.6 Hz, J=12.7 Hz, 1H), 3.83 (dd, J=2.3 Hz, J=12.7 Hz, 1H), 1.64 (s, 3H), 1.38 (s, 3H).

MS: calc 433.2; found 434.0 (MH⁺)

### Step 4: methyl 4-(((3aS,4S,6R,6aR)-6-(6-amino-2-iodo-9H-purin-9-yl)-2,2-dimethyl-tetrahydrofuro[3,4-d][1,3]dioxol-4-yl)methylthio)-2-(tert-butoxycarbonylamino)butanoate 58a

Sodium hydride (60% in mineral oil, 28 mg, 0.62 mmol) was added in one portion to a solution of 57a (150 mg, 0.346 mmol) in THF at 0° C. and the mixture stirred 30 min. TsCl (73 mg, 0.38 mmol) was added in one portion, the system warmed up to room temperature and stirred 1 h. The mixture was poured into EtOAc, washed and water, and the organic layer was separated, dried (Na₂SO₄) and concentrated and used without further purification. A mixture of tert-butyl 2-oxo-tetrahydrothiophen-3-ylcarbamate 4 (112 mg, 0.519 mmol) and sodium methoxide (0.5 N in MeOH, 1 mL) was stirred 1 for 10 min at room temperature, then it was added to the crude tosylate in MeOH (3.5 ml) and the resulting solution was refluxed for 1 h. The crude material was concentrated under reduced pressure; the residue dissolved in EtOAc, washed with water, dried (Na₂SO₄) and concentrated. The residue was purified by flash chromatography (pure EtOAc) giving the title compound 58a in 76% yield (175 mg) as clear oil. ¹H-NMR: CDCl₃ δ (ppm) 7.96 (s, 1H), 6.05 (d, H=1.7 Hz, 1H), 6.1 (d, J=6.1 Hz, 1H), 5.94 (dd, J=1.7 Hz, J=6.1 Hz, 1H), 5.06 (dd, J=3.3 Hz, J=6.6 Hz, 1H), 4.37 (m, 2H), 2.95 (ddd, J=1.5 Hz, J=7.4 Hz, J=13.5 Hz, 1H), 2.86 (ddd, J=1.5 Hz, J=6.3 Hz, J=13.5 Hz, 1H), 6.23 (m, 2H), 1.64 (s, 3H), 1.45 (s, 9K), 1.43 (s, 3H).

MS: calc 664.5; found 665.2 (MH⁺)

### Step 5: 2-amino-4-((2S,3S,4R,5R)-5-(6-amino-2-iodo-9H-purin-9-yl)-3,4-dihydroxy-tetrahydrofuran-2-yl)methylthio)butanoic acid 59a

A mixture of 58a (175.1 mg, 0.263 mmol), KOH (29.5 mg, 0.53 mmol), H₂O (1.75 mL) and THF (1.75 mL) was stirred for 1.5 h at room temperature (The reaction mixture turned homogeneous afterwards). The THF was removed under reduced pressure; water was added (10 mL) and the solution extracted with Et₂O. The aqueous solution was acidified (pH˜3) by addition of 1N HCl; the suspension extracted with DCM (2×), the combined organic extracts dried (Na₂SO₄) and concentrated. The residue was dissolved in DCM (2 mL), TFA (2 mL) was added at room temperature and the mixture stirred 1 h. H₂O (1 mL) was added and the mixture was stirred for one more hour. The solvent was removed under reduced pressure, the residue dissolved in water and lyophilized. The crude material was purified by preparative HPLC (MeOH 5% to 20% in water, in 35 min, C-18 reverse phase column) giving the title compound 59a in 7% yield as white solid (10 mg). ¹H-NMR: DMSO-d₆ δ (ppm) 8.31 (s, 1H), 7.73 (br, 2H), 5.80 (d, J=5.7 Hz, 1H), 5.64 (br, 1H), 4.69 (m, 1H), 4.14 (m, 1H), 4.10 (m, 1H), 4.03 (m, 1H), 3.2-3.8 (br, 2H), 2.93 (m, 1H), 2.80 (m, 1H), 2.70 (m, 2H), 2.14 (m, 1H), 1.9 (m, 1H).

MS: calc 510.5; found 511.0 (MH⁺)

### EXAMPLE 85

### 2-amino-4-(((2S,3S,4R,5R)-5-(6-amino-2-bromo-9H-purin-9-yl)-3,4-dihydroxy-tetrahydrofuran-2-yl)methylthio)butanoic acid 59b

The title compound 59b, was prepared in 5.4% yield as light brown solid in a manner similar to example 84, scheme 9, replacing I₂ with NBS in step 1. ¹H-NMR: DMSO-d₆ δ (ppm) 8.38 (s, 1H), 7.86 (br, 2H), 5.81 (d, J=4.8 Hz, 1H), 4.63 (m, 1H), 4.10 (m, 1H), 4.03 (m, 1H), 3.2 (br, 3H), 2.9 (m, 1H), 2.82 (m, 1H), 2.63 (m, 1H), 2.24 (m, 1H), 1.86 (m, 1H).

MS: calc 464 (100%), 462 (97%); found 463.1 (100%), 465.1 (97%) (MH⁺)

### EXAMPLE 86

### 2-amino-4-(((2S,3S,4R,5R)-5-(6-amino-2-fluoro-9H-purin-9-yl)-3,4-dihydroxy-tetrahydrofuran-2-yl)methylthio)butanoic acid 59c

The title compound 59c, was prepared in a manner similar to example 84, scheme 9, with the following modifications to step 1. XeF₂ (524 mg, 2.04 mmol) was added in one portion to a solution of 54 (500 mg, 0.68 mmol), 2,6-di-tert-butyl-4-methylpyridine (140 mg, 0.68 mmol) and AgTfO (542 mg, 2.04 mmol) in DCM and the mixture stirred 15 min at room temperature. The reaction mixture was poured over NaHCO₃ (aq. sat. solution) and extracted with DCM (2×). The combined organic layers were filtered through Celite™ and concentrated. The residue was dissolved in DCM, filtered again and concentrated. The crude was purified by flash chromatography (EtOAc/Hex 1:5) giving 55c in 48% yield (150 mg) as a yellow syrup. MS: calc 458.16 (100%), 460.15 (35%); found 459.3 (100%) 461.3 (34%) (MH⁺)

All other steps are the same as described for example 84. The title compound 59c, was obtained in 15% yield as white solid. ¹H-NMR: DMSO-d₆ δ (ppm) 8.32 (s, 1H), 8.12 (s, 1H), 7.80 (br, 2H), 6.56 (s, 1H), 5.75 (d, J=4.8 Hz, 1H), 5.72 (br, 1H), 4.68 (m, 1H), 4.52 (m, 1H), 4.20 (m, 2H), 4.05 (m, 1H), 3.92 (m, 1H), 2.80 (br, 2H), 2.70 (m, 2H), 2.00 (m, 1H), 1.80 (m, 1H).

MS: calc 402.11; found 403.4 (MH⁺)

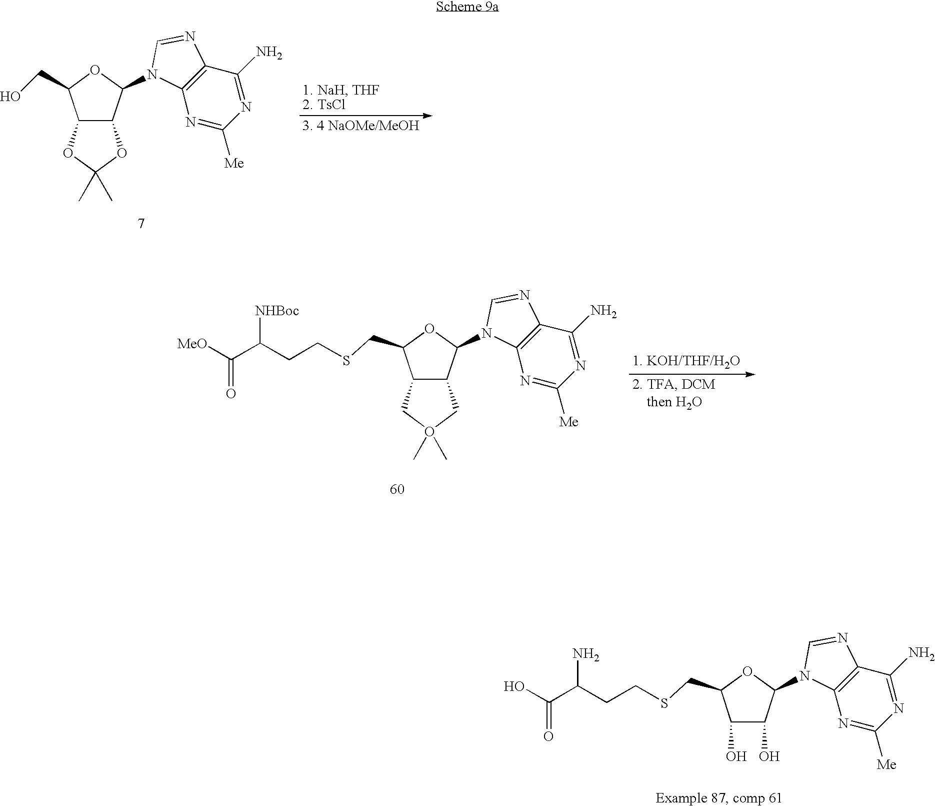

### EXAMPLE 87

### 2-amino-4-(((2S,3S,4R,5R)-5-(6-amino-2-methyl-9H-purin-9-yl)-3,4-dihydroxy-tetrahydrofuran-2-yl)methylthio)butanoic acid 61

### Step 1: methyl 4-(((3aS,4S,6R,6aR)-6-(6-amino-2-methyl-9H-purin-9-yl)-2,2-dimethyl-tetrahydrofuro[3,4-d][1,3]dioxol-4-yl)methylthio)-2-(tert-butoxycarbonylamino)butanoate 60

The title compound 60 was prepared following the procedure detailed for example 84, scheme 9, steps 4, replacing 57a with ((3aR,4R,6R,6aR)-6-(6-amino-2-methyl-9H-purin-9-yl)-2,2-dimethyl-tetrahydrofuro[3,4-d][1,3]dioxol-4-yl)methanol 7 (51 mg, 0.16 mmol, prepared according to the procedure of Yamazaki et al, J. Org. Chem. 1968, 33, 2583). MS calc 552.64; found 553.2 (MH⁺)

### Step 2: 2-Amino-4-(((2S,3S,4R,5R)-5-(6-amino-2-methyl-9H-purin-9-yl)-3,4-dihydroxy-tetrahydrofuran-2-yl)methylthio)butanoic acid 61

The title compound 61, was obtained as white solid in 81% yield (30 mg) utilizing the procedures detailed in scheme 9 for example 84, in step 5, replacing 58a, with 60. ¹H NMR (CD₃OD): ppm: 8.32(s, 1H), 6.06(d, 1H, J=4.8, 1-H), 4.80(m, 1H), 4.68 (s, 1H), 4.40(m, 1H), 4.30 (m, 1H), 3.74 (m, 1H), 3.08 (m, 2H), 2.80(m, 2H), 2.58(s, 3H, Me), 2.40(m, 1H), 2.10(m, 1H).

MS: calc 398.44; found 399.4(MH⁺)

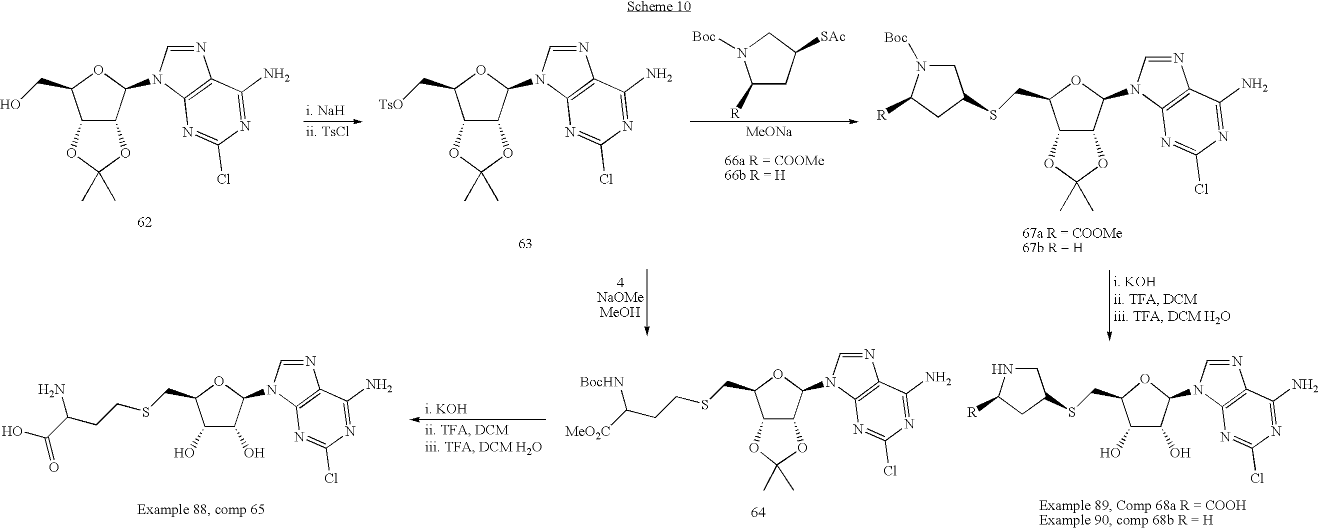

### EXAMPLE 88

### 2-amino-4-(((2S,3S,4R,5R)-5-(6-amino-2-chloro-9H-purin-9-yl)-3,4-dihydroxy-tetrahydrofuran-2-yl)methylthio)butanoic acid 65

### Step 1: methyl 4-(((3aS,4S,6R,6aR)-6-(6-amino-2-chloro-9H-purin-9-yl)-2,2-dimethyl-tetrahydrofuro[3,4-d][1,3]dioxol-4-yl)methylthio)-2-(tert-butoxycarbonylamino)butanoate 64

Compound 64 was prepared in quantitative yield as a clear syrup following the procedure detailed in step 4 for compound 58a, example 84, scheme 9, replacing 57a with 62 (prepared according to the method of Andrzejewska, Mariola; Kaminski, Jaroslaw; Kazimierczuk, Zygmunt Nucleosides Nucleotides 2002, 21 (1), 73-78).

MS: calc 572.18 (100%, 574.18 (37%); found 573.3 (100%), 575.2 (41%) (MH⁺)

### Step 2: 2-amino-4-(((2S,3S,4R,5R)-5-(6-amino-2-chloro-9H-purin-9-yl)-3,4-dihydroxy-tetrahydrofuran-2-yl)methylthio)butanoic acid 65

The title compound 65 was prepared in 14% yield as white solid following the procedure detailed in step 5 for compound 59a, example 84, scheme 9, and replacing 58a with 64. ¹H-NMR: DMSO-d₆ δ (ppm) 8.4 (s, 1H), 7.86 (br, 2H), 5.82 (d, J=6.2 Hz, 1H), 4.63 (m, 1H), 4.14 (m, 1H), 4.10 (m, 1H), 3.2-3.8 (br, 3H), 2.9 (m, 1H), 2.80 (m, 1H), 2.62 (m, 2H), 2.26 (m, 1H), 1.86 (m, 1H).

MS: calc 418.08 (100%), 420.08 (37%); found 419.2 (100%), 421.1 (38%) (MH⁺)

### EXAMPLE 89

### (2S,4R)-4-(((2S,3S,4R,5R)-5-(6-amino-2-chloro-9H-purin-9-yl)-3,4-dihydroxy-tetrahydrofuran-2-yl)methylthio)pyrrolidine-2-carboxylic acid 68a

### Step 1: (2S,4R)-methyl 4-(((2S,3S,4R,5R)-5-(6-amino-2-chloro-9H-purin-9-yl)-3,4-dihydroxy-tetrahydrofuran-2-yl)methylthio)pyrrolidine-2-carboxylate 67a

Compound 67a was prepared in 47% yield as a clear syrup following the procedure detailed in step 4 for compound 58a, example 84, scheme 9, replacing 57a with (2S,4S)-1-tert-butyl 2-methyl 4-(acetylthio)pyrrolidine-1,2-dicarboxylate 66a.

### Step 2: (2S,4R)-4-(((2S,3S,4R,5R)-5-(6-amino-2-chloro-9H-purin-9-yl)-3,4-dihydroxy-tetrahydrofuran-2-yl)methylthio)pyrrolidine-2-carboxylic acid 68a

The title compound 68a, was prepared in 36% yield as white solid following the procedure detailed in step 5 for compound 59a, example 84, scheme 9, replacing 58a with 67a. ¹H NMR (D₂O) δ (ppm) 7.88 (s, 1H), 5.61 (d, 1H, J=4.5 Hz, 1H), 4.46 (dd, J=4.5 Hz, J=5 Hz, 1H), 4.14 (dd, J=5 Hz, J=5.3 Hz, 1H), 4.05 (m, 2H), 3.45 (m, 2H), 3.16 (m, 1H), 2.88 (dd, J=14 Hz, J=5.1 Hz, 1H), 2.78 (dd, J=14 Hz, J=6.1 Hz, 1H), 2.57 (m, 1H).

MS: calc 430.08 (100%), 432.08 (37%); found 431.3 (100%), 433.3 (17%) (MH⁺)

### EXAMPLE 90

### (2R,3R,4S,5S)-2-(6-amino-2-chloro-9H-purin-9-yl)-5-(((R)-pyrrolidin-3-ylthio)methyl)-tetrahydrofuran-3,4-diol 68b

### Step 1: (R)-tert-butyl 3-(((3aS,4S,6R,6aR)-6-(6-amino-2-chloro-9H-purin-9-yl)-2,2-dimethyl-tetrahydrofuro[3,4-d][1,3]dioxol-4-yl)methylthio)pyrrolidine-1-carboxylate 67b

Compound 67b was prepared in 33% yield as a clear syrup following the procedure detailed in step 4 for 58a, example 84, scheme 9, replacing 57a with (S)-tert-butyl 3-(acetylthio)pyrrolidine-1-carboxylate 66b.

MS: Calc 526.18 (100%), 528.17 (37%); found 565.2 (100%), 567.2 (45%) (MH⁺)

### Step 2: (2R,3R,4S,5S)-2-(6-amino-2-chloro-9H-purin-9-yl)-5-(((R)-pyrrolidin-3-ylthio)methyl)-tetrahydrofuran-3,4-diol 68b

The title compound 68b was prepared in 12% yield as white solid following the procedure detailed in step 5 for compound 59a, example 84, scheme 9 and replacing 58a with 67b. ¹H NMR (D₂O) δ (ppm) 8.26 (br, 1H), 8.08 (s, 1H), 5.8 (d, 1H, J=4.7 Hz, 1H), 4.7 (dd, J=4.7 Hz, J=5.1 Hz, 1H), 4.27 (dd, J=5.1 Hz, J=5.1 Hz, 1H), 4.16 (m, 1H), 3.48 (m, 1H), 3.33 (m, 1H), 3.33 (m, 2H), 3.21 (m, 1H), 3.02 (dd, J=4.9 Hz, J=12.1 Hz, 1H), 2.26 (dd, J=4.5 Hz, J=14.1 Hz, 1H), 2.89 (dd, J=7 Hz, J=14.1 Hz, 1H), 2.20 (m, 1H), 1.82 (m, 1H)

MS: calc 386.09 (100%), 388.09 (37%); found 387.2 (100%), 389.1 (40%) (MH⁺)

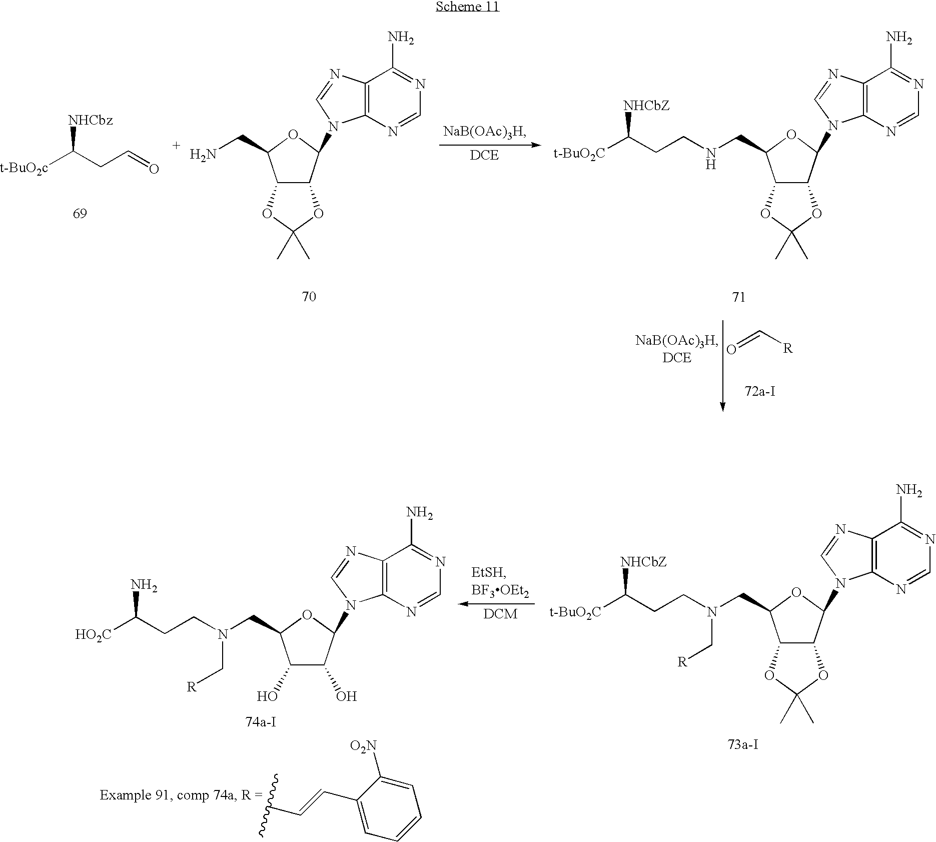

### EXAMPLE 91

### (S)-4-(N-(2-nitrocinnamyl)-N-(((2R,3S,4R,5R)-5-(6-amino-9H-purin-9-yl)-tetrahydro-3,4-dihydroxyfuran-2-yl)methyl)amino)-2-aminobutanoic acid 74a

### Step 1: benzyl (S)-3-(((3aR,4R,6R,6aR)-4-(6-amino-9H-purin-9-yl)-tetrahydro-2,2-dimethylfuro[3,4-d][1,3]dioxol-6-yl)methylamino)-1-(tert-butoxycarbonyl)propylcarbamate 71

To a solution of (S)-tert-butyl 2-(benzyloxycarbonylamino)-4-oxobutanoate 69 (4.0 ml, 1.41 mmol, prepared according to I. Weits et al, J. Org. Chem. 1997, 62, 2527-2534) in dichloroethane (7.5 ml) at room temperature was added to 9-((3aR,4R,6R,6aR)-6-(aminomethyl)-2,2-dimethyl-tetrahydrofuro[3,4-d][1,3]dioxol-4-yl)-9H-purin-6-amine 70 (538.0 mg, 1.76 mmol, prepared according to A. M. Reeve et al, Tetrahedron 1998, 54, 15959-15974). The mixture was heated slightly until it became homogeneous and then was allowed to cool to room temperature. Sodium triacetoxyborohydride (418.0 mg, 1.97 mmol) was added portion wise and the mixture was stirred for 2 h. The reaction was quenched with aqueous sodium bicarbonate and then the aqueous layer was extracted repeatedly with dichloromethane. The organic washings were dried over sodium sulfate, filtered and concentrated under reduced pressure. Column chromatography using a solvent gradient of ethyl acetate to 10%MeOH/EtOAc yielded the title compound 71 as white solid (538 mg, 64%). ¹H NMR: (300 MHz, DMSO-d₆) δ (ppm): 8.39 (1H, s), 8.22 (1H, s), 7.70 (1H, d, J=7.7), 7.40-7.35 (7H, m), 6.12 (1H, d, J=3.3), 5.50 (1H, dd, J=6.2, 3.5), 5.08 (2H, s), 5.02 (1H, dd, J=6.3, 2.5), 4.25 (1H, m), 4.10 (1H, m), 2.80 (1H, m), 2.69 (1H, m), 2.62-2.57 (2H, m), 2.08 (1H, m), 1.84 (1H, m), 1.73 (1H, m), 1.59 (3H, s), 1.43 (9H, s), 1.36 (3H, s). MS calc.: 597.66 observed.: 598.2

### Step 2: benzyl (S)-1-(tert-butoxycarbonyl)-3-(N-(2-nitrocinnamyl)-N-(((3aR,4R,6R,6aR)-4-(6-amino-9H-purin-9-yl)-tetrahydro-2,2-dimethylfuro[3,4-d][1,3]dioxol-6-yl)methyl)amino)propylcarbamate 73a

To a solution of amine 71 (300.0 mg, 0.502 mmol) in dichloroethane (4 ml) at room temperature was added (E)-3-(2-nitrophenyl)acrylaldehyde 72a (156.0 mg, 0.88 mmol). The mixture was allowed to stir at rt for 15 min. then sodium triacetoxyborohydride (150.0 mg, 0.703 mmol) was added and the mixture was stirred for 3 h. The reaction was quenched with aqueous sodium bicarbonate, allowed to stir vigorously for 30 min. and then the aqueous layer was extracted repeatedly with dichloromethane. The organic extracts were dried over sodium sulfate, filtered and concentrated under reduced pressure. Column chromatography using a solvent gradient of ethyl acetate to 5%MeOH/EtOAc yielded the title compound 73a as white solid (376 mg, 98%). ¹H NMR: (300 MHz, DMSO-d₆) δ (ppm): 8.37 (1H, s), 8.15 (1H, s), 7.97 (1H, d, J=8.0), 7.76-7.67 (2H, m), 7.63 (1H, d, J=7.7), 7.54 (1H, m), 7.39-7.34 (7H, m), 6.83 (1H, d, J=16.0), 6.34 (1H, m), 6.21 (1H, d, J=2.2), 5.54 (1H, dd, J=6.2, 2.3), 5.09-5.05 (2H, m), 4.92 (1H, d, J=12.6), 4.34 (1H, m), 4.12 (1H, m), 3.39 (1H, m), 3.23 (1H, dd, J=14.7, 7.9), 2.64-2.60 (2H, m), 2.50 (1H, m). MS calc: 758.8 observed.: 759.3

### Step 3: (S)-4-(N-(2-nitrocinnamyl)-N-(((2R,3S,4R,5R)-5-(6-amino-9H-purin-9-yl)-tetrahydro-3,4-dihydroxyfuran-2-yl)methyl)amino)-2-aminobutanoic acid (74a)

To a solution of EtSH (0.44 ml, 5.94 mmol)) and BF₃.OEt₂ (0.25 ml, 1.98 mmol) in dichloromethane (1.5 ml) at 0° C. was added a solution of 73a (150 mg, 0.20 mmol) in dichloromethane (0.5 ml) and then a cap was placed tightly on top. After 30 min. the mixture was allowed to warm to rt, the cap removed and mixture vented and then the cap was placed tightly on top again. After 18 h. the mixture was concentrated, re-dissolved in acetone/H₂O and concentrated again. The crude was purified by column chromatography (CHCl₃/MeOH/NH₄OH, 6:3:1) and then Dowex 50WX8-200 resin to afford the title compound 74a as an off-white solid in 36% yield (38 mg). ¹H NMR: (300 MHz, DMSO-d₆) δ (ppm): 8.22 (1H, s), 8.13 (1H, s), 7.97 (1H, d, J=8.2), 7.65 (1H, m), 7.57-7.49 (2H, m), 7.01 (1H, d, J=15.9), 6.25 (1H, m), 6.05 (1H, d, J=3.8), 4.76 (1H, m), 4.48 (1H, t, J=5.6), 4.40 (1H, m), 3.74 (1H, dd, J=9.4, 2.9), 3.57 (1H, dd, J=15.7, 5.5), 3.34 (1H, m), 3.15 (1H, dd, J=13.3, 9.8), 3.03 (1H, m), 2.94-2.85 (2H, m), 2.21 (1H, m), 2.00 (1H, m).

Examples 92, compound 74b, Table 8, was prepared according to the procedure described for example 91, in step 2 using 72b, 3-(pyridin-3-yl)propanal (prepared according to the procedure of M. Stocks et al, Tet. Let. 1995, 36, 6555-8) in place of aldehyde 72a.

Example 93, compound 74c, Table 8, was prepared according to the procedure described for example 91, in step 2 using 72c, 3-(2-nitrophenyl)propanal (previously reported by Harmon et al. J. Org. Chem. 1969, 34, 3684; however, it was prepared according to the method in JOC, 1992, 57(11), 3218-3225) in place of aldehyde 72a.

Example 94, compound 74d, Table 8, was prepared according to the procedure described for example 91, in step 2 using 72d, 4-nitrobutanal (previously reported by R. Kimura et al. Bull. Chem. Soc. Jpn., 2002, 75(11), 2517-2526, however it was prepared in 94% yield by DIBAL-H reduction of methyl 4-nitrobutanoate in DCM at −78° C.) in place of aldehyde 72a.

Examples 95-102, compounds 74e-74l, Table 8, were prepared according to the method described for example 91, utilizing commercially available aldehydes 72e-72l.

TABLE 8

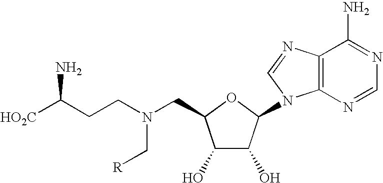

| ex | cpd | R—X | Name | Characterization | Scheme |
|---|---|---|---|---|---|
| 92 | 74b | 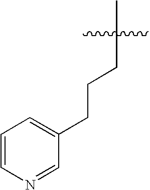 | (S)-4-(N-(((2R,3S,4R,5R)-5-(6-ami-no-9H-purin-9-yl)-tetra-hydro-3,4-di-hydroxyfuran-2-yl)meth-yl)-N-(3-(pyri-din-3-yl)pro-pyl)amino)-2-amino)-butanoic acid | 1H NMR: (300 MHz, CD₃OD)δ(ppm): 8.43–8.40(2H, m), 8.32(1H,s), 8.27(1H, s), 7.66(1H, d,J=7.7),7.38(1H, dd, J=7.8, 4.8),6.08(1H, d, J=4.4), 4.84(2H, t,J=4.7), 4.51–4.44(2H, m), 3.83(1H,dd, J=9.1, 3.8), 3.71–3.64(1H,m), 3.53(1H, d, J=14.0),3.50–3.40(1H, m), 3.30–3.12(2H,m), 2.77–2.68(2H, m), 2.09–2.03(3H,m) MS calc.: 486.5observed.: 487.3 | 11 |
| 93 | 74c | 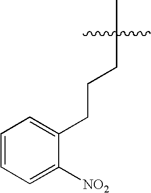 | (S)-4-(N-(((2R,3S,4R,5R)-5-(6-ami-no-9H-purin-9-yl)-tetra-hydro-3,4-dihydroxy-furan-2-yl)meth-yl)-N-(3-(2-nitro-phenyl)propyl)ami-no)-2-amino-butanoic acid | ¹H NMR(400 MHz, CD₃OD)δ(ppm): 8.38(s, 1H), 8.18(s,1H), 7.94(d, J=8.35, 1H), 7.63(t,J=7.47,1H), 7.48(dd, J=7.91,7.47, 1H), 7.43(d, J=7.91, 1H),7.33(brs, 2H), 5.93(d, J=4.84,1H), 5.52(br, 1H), 4.70(brm,1H), 4.20(m, 1H), 4.08(m, 1H),2.85–2.64(m, 6H), 1.96(brm,1h), 1.75(brm, 3H) MS calc.:530.54 observed.: 531.5 | 11 |
| 94 | 74d | 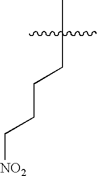 | (S)-4-(N-(((2R,3S,4R,5R)-5-(6-ami-no-9H-purin-9-yl)-tetra-hydro-3,4-dihydroxy-furan-2-yl)meth-yl)-N-(4-nitro-butyl)amino)-2-amino-butanoic acid | ¹H NMR(400 MHz, CD₃OD)δ(ppm): 8.23(s, 1H), 8.22(s,1H), 5.99(d, J=4.40, 1H), 4.77(t,J=4.8 1H), 4.34(t, J=5.2,1H),4.30(m, 1H), 3.67(t, J=6.2, 1H),3.01(brm, 2–3H), 2.81(brm, 2H),2.14(m, 1H), 1.92(m, 2–3H),1.62(m, 2H) MS calc.: 468.47observed.: 469.4 | 11 |
| 95 | 74e | 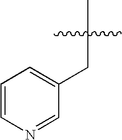 | (S)-4-(N-(((2R,3S,4R,5R)-5-(6-ami-no-9H-purin-9-yl)-tetra-hydro-3,4-dihydroxy-furan-2-yl)meth-yl)-N-((pyridin-3-yl)meth-yl)amino)-2-amino-butanoic acid | 1H NMR: (300 MHz, CD₃OD) δ(ppm): 8.54(1H, d, J=1.4), 8.50(1H,dd, J=4.9, 1.7), 8.21(1H, s),8.17(1H, s), 7.84(1H,m), 7.34(1H,dd, J=7.1, 4.9), 6.03(1H, d,J=4.1), 4.72(1H, t, J=4.5), 4.36–4.31(2H,m), 3.93(1H, d,J=14.0), 3.75(1H, d, J=14.0),3.68(1H, dd, J=8.8, 3.6), 2.98–2.82(4H,m), 2.23(1H, m), 2.04(1H,m) MS calc.: 458.5observed.: 459.1 | 11 |
| 96 | 74f | 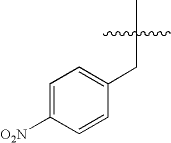 | (S)-4-(N-(4-nitro-benzyl)-N-(((2R,3S,4R,5R)-5-(6-ami-no-9H-purin-9-yl)-tetra-hydro-3,4-dihydroxy-furan-2-yl)meth-yl)amino)-2-amino-butanoic acid | ¹H NMR(400 MHz, CD₃OD) δ(ppm): 8.11(d, J=8.4, 2H), 7.97(s,1H), 7.94(s, 1H), 7.44(d,J=8.4, 2H), 6.03(d, J=3.5,1H),4.76(m, 1H), 4.46–4.37(m, 2H),4.03(d, J=14.07, 1H), 3.83(dd,J=8.79, 3.52, 1H), 3.67(d,J=14.07 1H), 3.03–2.91(m, 2H),2.84(d, J=5.7, 2H), 2.21(m, 1H),2.10(m, 1H) MS calc.: 502.49observed.: 503.4 | 11 |
| 97 | 74g | 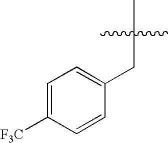 | (S)-4-(N-(4-(tri-fluoromethyl)ben-zyl)-N-(((2R,3S,4R,5R)-5-(6-ami-no-9H-purin-9-yl)-tetra-hydro-3,4-dihydroxy-furan-2-yl)meth-yl)amino)-2-amino-butanoic acid | ¹H NMR(400 MHz, CD₃OD)δ(ppm): 8.33(s, 1H), 8.15(s,1H), 7.65(d, J=7.91, 2H), 7.56(d,J=7.91,2H), 7.34(s, 2H), 5.92(d,J=4.84, 1H), 4.65(m, 1H),4.17(m, 2H), 3.78(dd, J=23.74,14.07, 2H), 3.37(br, 2H), 2.88(m,1H), 2.75–2.66(m, 2H), 2.04(brm,1H), 1.85(brm, 1H)MS calc.: 525.49; found.: 526.3 | 11 |
| 98 | 74h | 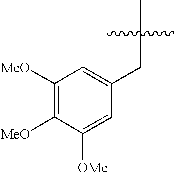 | (S)-4-(N-(3,4,5-tri-methoxybenzyl)-N-(((2R,3S,4R,5R)-5-(6-ami-no-9H-pur-in-9-yl)-tetra-hydro-3,4-dihydroxy-furan-2-yl)meth-yl)amino)-2-amino-butanoic acid | ¹H NMR(400 MHz, CD₃OD)δ(ppm): 8.32(s, 1H), 8.16(s,1H), 7.34(brs, 2H), 6.67(s, 2H),5.93(d,J=4.84, 1H), 5.66(br,1H), 4.63(br, 1H), 4.17(brs, 2H),3.74(s, 6H), 3.68(s, 3H), 3.40–3.35(m,6H), 2.90(m, 1H), 2.75–2.68(m,3H), 2.05(m, 1H), 1.85(m,1H) MS calc.: 547.57; found.:548.3 | 11 |
| 99 | 74i | 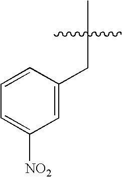 | (S)-4-(N-(3-nitro-benzyl)-N-(((2R,3S,4R,5R)-5-(6-ami-no-9H-purin-9-yl)-tetra-hydro-3,4-dihydroxy-furan-2-yl)meth-yl)amino)-2-amino-butanoic acid | ¹H NMR(400 MHz, CD₃OD)δ(ppm): 8.20–8.09(m, 4H), 7.73(d,J=7.47, 1H), 7.49(t, J=7.91,1H), 6.03(d, J=3.52,1H), 4.69(m,1H), 4.37(m, 2H), 4.01(d,J=14.07, 1H), 3.79(d, J=14.07,1H), 3.70(m, 1H), 3.0–2.84(m,4H), 2.22(m, 1H), 2.05(m, 1H)MS calc.: 502.49; found.: 503.4 | 11 |
| 100 | 74j | 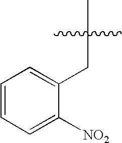 | (S)-4-(N-(2-nitro-benzyl)-N-(((2R,3S,4R,5R)-5-(6-ami-no-9H-purin-9-yl)-tetra-hydro-3,4-di-hydroxyfuran-2-yl)meth-yl)amino)-2-amino-butanoic acid | ¹H NMR(400 MHz, CD₃OD)δ(ppm): 8.31(d, J=7.47, 1H),8.18(s, 1H), 8.17(s, 1H), 7.79(m,1H), 7.65(d, J=7.03,1H),7.47(m, 1H), 5.98(d, J=3.96,1H), 4.70(t, J=4.40, 1H), 4.30(m,2H), 4.19(d, J=14.51, 1H),3.90(d, J=14.95, 1H), 3.59(dd,J=8.79, 3.96, 1H), 2.94–2.83(m,4H), 2.16(m, 1H). MS calc.:502.49; found.: 503.4 | 11 |
| 101 | 74k | 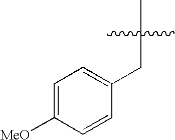 | (S)-4-(N-(4-meth-oxybenzyl)-N-(((2R,3S,4R,5R)-5-(6-ami-no-9H-purin-9-yl)-tetra-hydro-3,4-dihydroxy-furan-2-yl)meth-yl)amino)-2-amino-butanoic acid | ¹H NMR(400 MHz, CD₃OD)δ(ppm): 8.16(s, 1H), 8.06(s,1H), 7.18(d, J=8.61, 2H), 6.79(d,J=8.22, 2H), 5.97(d,J=3.91,1H), 4.63(t, J=4.5, 1H), 4.32(m,1H), 4.27(m, 1H), 3.88(d,J=12.91, 1H), 3.77(s, 3H), 3.52(m,1h), 3.07–2.77(m, 5H), 2.11(m,1H), 1.94(m, 1H) MS calc.:487.51; found: 488.5 | 11 |
| 102 | 74l | 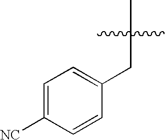 | (S)-4-(N-(4-cyano-benzyl)-N-(((2R,3S,4R,5R)-5-(6-ami-no-9H-purin-9-yl)-tetra-hydro-3,4-dihydroxy-furan-2-yl)meth-yl)amino)-2-amino-butanoic acid | ¹H NMR(400 MHz, CD₃OD)δ(ppm): 8.12(s, 1H), 8.10(s,1h), 7.53(d, J=7.83, 2H), 7.43(d,J=7.83, 2H), 5.95(d,J=3.91,1H), 4.65(m, 1H), 4.28(m, 2H),3.89(d, J=14.09, 1H), 3.68(d,J=14.09, 1H), 3.59(m, 1H), 2.86–2.76(m,4H), 2.13(m, 1H), 1.95(m,1H). MS calc.: 482.50;found.: 483.5 | 11 |

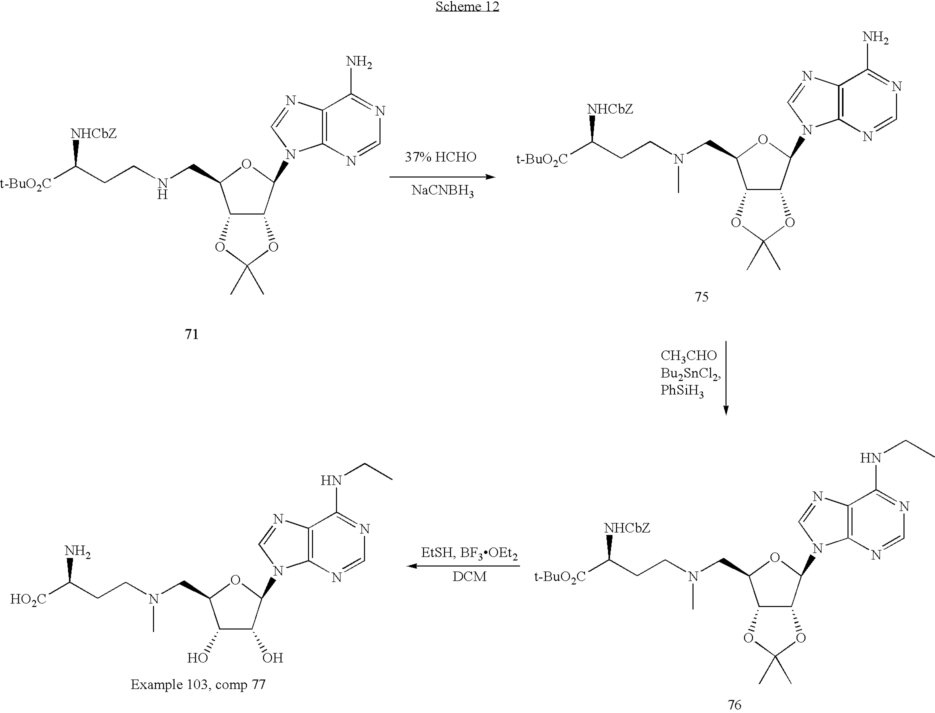

### EXAMPLE 103

### (S)-4-(N-(((2R,3S,4R,5R)-5-(6-(ethylamino)-9H-purin-9-yl)-tetrahydro-3,4-dihydroxyfuran-2-yl)methyl)-N-methylamino)-2-aminobutanoic acid 77

### Step 1: (S)-tert-butyl 4-((((3aR,4R,6R,6aR)-6-(6-amino-9H-purin-9-yl)-2,2-dimethyl-tetrahydrofuro[3,4-d][1,3]dioxol-4-yl)methyl)(methyl)amino)-2-(benzyloxycarbonylamino)butanoate 75

To a solution of amine 71, scheme 11 (0.75 g, 1.25 mmol) in DOE (dichloroethane) at rt was added formaldehyde (37% in H2O, 0.14 ml, 1.9 mmol). To this emulsion was added sodium cyanoborohydride (0.37, 1.75 mmol) and the resulting mixture was stirred 1 h. When reaction was complete, solvent was removed in vacuo and the crude residue was purified by column chromatography to afford the title compound 75 in 50% yield as a solid. MS calc.: 611.69, observed: 612.5.

### Step 2: benzyl (S)-1-(tert-butoxycarbonyl)-3-(N-(((3aR,4R,6R,6aR)-4-(6-(ethylamino)-9H-purin-9-yl)-tetrahydro-2,2-dimethylfuro[3,4-d][1,3]dioxol-6-yl)methyl)-N-methylamino)propylcarbamate 76

Acetaldehyde (0.05 ml, 0.90 mmol), Bu₂SnCl₂ (12.0 mg, 0.039 mmol) was added to a solution of 75 (250.0 mg, 0.409 mmol) in THF (0.75 ml) was added and then PhSiH₃ (0.10 ml, 0.82 mmol) quickly and the reaction flask sealed tightly. After 18 h. the reaction mixture was concentrated and the crude was purified by column chromatography (2%MeOH/EtOAc) to afford the title compound 76 in 34% yield as an off-white solid (89 mg). MS calc: 639.7; found: 640.5.

### Step 3: (S)-4-(N-(((2R,3S,4R,5R)-5-(6-(ethylamino)-9H-purin-9-yl)-tetrahydro-3,4-dihydroxyfuran-2-yl)methyl)-N-methylamino)-2-aminobutanoic acid 77

Following the procedure for compounds described for example 91, the title compound 77 was obtained as a white solid in 56% yield (9 mg). ¹H NMR: (400 MHz, CD₃OD) δ(ppm): 8.26 (s, 1H), 8.22 (s, 1H), 5.98 (d, J=4.70, 1H), 4.74 (t, J=5.09, 1H), 4.26 (m, 1H), 4.21 (t, J=5.28, 1H), 3.63 (dd, J=9.0, 3.13, 2H), 2.93 (dd, J=13.11, 9.59, 1H), 2.82-2.65 (m, 3-4H), 2.08 (m, 1H), 1.92 (m, 1H), 1.32 (t, J=7.24, 3H), MS calc: 409.44; found: 410.2

### ASSAY EXAMPLES

### ASSAY EXAMPLE 1

### Inhibition of DNMT1 and DNMT3b2 Activity

The following protocols were used to assay the compounds of the invention.

Human DNA Methyltransferases (hDNMTs) Cloning, Expression and Purification

### Construction of Baculoviruses

A 5.0-kb cDNA corresponding to the full length DNMT1 (Swissprot accession number P26358 (SEQ ID NO.1)) was cloned into the BamHI/SalI site of the pBlueBac4.5 vector (Invitrogen) Also, a 2.7-kb cDNA encoding the DNMT3 splice variant 2 of DNMT3b2 (Swissprot accession number Q9UBC3-2 (SEQ ID NO.2)) was cloned into the BamHI/XbaI site of the abovementioned vector. These constucts were used to generate recombinant baculoviruses using the Bac-N-Blue™ DNA according to the manucfacturer's instructions (Invitrogen).

### Protein Expression

Full length hDNMTs proteins were expressed in Hi-5 cells (Trichoplusia Ni) upon infection with recombinant baculoviruses constructs. Briefly, Hi-5 cells grown in suspension and maintained in serum-free medium (Sf900 II supplemented with gentamycin) were infected with the abovementioned viruses at multiplicity of infection (MOI) varying from 1 to 3 during 84 hours at 27° C. with agitation at 120 rpm on a rotary shaker. Infected cells were harvested by centrifugation at 398 g for 15 min. after which a nuclear and cytosolic fractionation was performed. Fractions were frozen at −80° C. until purifications were performed.

### Nuclear Extraction

After harvesting of Hi-5 cells infected with the DNMT-1 recombinant baculovirus, cell pellets were gently resuspended in Buffer A (10 mM Tris pH 8.0, 1.5 mM MgCl₂, 5 mM KCl, 5 mM DTT, 0.5% NP-40, 25% glycerol, 1 μg/ml pepstatin, 2 μg/ml Aprotinin and leupeptin, 50 μg/ml PMSF, 50 μg/ml TLCK and 10 μM E64) and left on ice for 10 min. Nuclei were pelleted down at 500 g and supernatant (cytoplasmic fraction) kept on ice. Nuclei pellets were resuspended in Buffer B (20 mM Tris pH 8.0, 1.5 mM MgCl₂, 5 mM KCl, 5 mM DTT, 0.5% NP-40, 25% glycerol, 0.2 mM EDTA, 400 mM NaCl, 1 μg/ml pepstatin, 2 μg/ml Aprotinin and leupeptin, 50 μg/ml PMSF, 50 μg/ml TLCK and 10 μM E64) and left on ice for 45 min. followed by a centrifugation at 30000 g for 30 min. The supernatant was recovered as the nuclear fraction.

The nuclear extraction of Hi-5 cells infected with the DNMT3b2 recombinant baculovirus construct was performed as described above with the following modifications in buffers composition: Buffer A did not have glycerol and DTT. Also, glycerol concentration in Buffer B was 10%.

### Purification

All the purification steps described below were performed at 4° C. For DNMT-1 purification, both cytoplasmic and nuclear fractions were pooled together and NaCl concentration was adjusted to 0.1M by diluting with Buffer C (20 mM Tris pH 7.4, 10% sucrose and 1 mM EDTA) This material was centrifuged for 10 min. at 30000 g and the supernatant was loaded onto a QsepharoseFF column (Amersham Biosciences) equilibrated with Buffer C+0.1M NaCl. Following a ten column volume (CV) wash with equilibration buffer, bound proteins were eluted with a 10 CV salt linear gradient spanning from 0.1 to 1M NaCl in Buffer C. DNMT-1 containing fractions were pooled together. Typically, the conductivity of selected fractions ranked between 13.5 and 18.6 mS/cm. This Qsepharose eluate was diluted 4-fold with Buffer C and centrifuged as described above. The supernatant was applied to a Hitrap Heparin column (Amersham Biosciences) equilibrated with Buffer C+0.1M NaCl. Column was washed with 10 CV of equilibration buffer and proteins were eluted with a 10 CV salt linear gradient (0.1 to 1.5M NaCl in buffer C) The DNMT-1 enriched fractions were pooled according to SDS-PAGE analysis (coomassie staining) Final DNMT-1 protein preparations concentration were about 7 mg/ml and purity above 95%.

DNMT3b2 was purified using only the nuclear extract as starting material. The NaCl concentration of the latter was adjusted to 0.2M by diluting with Buffer D (50 mM NaPO₄ pH 7.8, 10% glycerol and 1 mM EDTA) supplemented with the protease inhibitors described in Buffer A followed by centrifugation at 30000 g for 10 min. The supernatant was loaded onto a Hitrap SPsepharose column (Amersham Biosciences) equilibrated with Buffer D with 0.05M NaCl. Column was washed with 8 CV of equilibration buffer and proteins were eluted with an 8 CV linear gradient of NaCl (from 0.05 to 1M) in Buffer D. DNMT3b2 containing fractions were pooled based on SDS-PAGE analysis. Selected fractions from this elution had conductivity varying from 15.1 to 28.2 mS/cm. Finally, this SPsepharose eluate underwent buffer exchange, using PD-10 column (Amersham Biosciences) against Buffer D+0.3M NaCl. Typical DNMT3b2 enzyme preparation had concentration of about 2.5 mg/ml and approximately 70% purity.

Purified DNMT-1 and DNMT3b2 protein stocks were aliquoted and frozen at −80° C. prior to use in enzymatic assay.

### DNA Methyltransferase Assay

### Reagents:

Enzyme: Cloned human DNA Methyltransferase (DNMT1 and DNMT3b2)

Expressed in insect cells and purified at 98%

### Substrate 1:

Oligonucleotides

MYG167:
ATC GCA TCG ATC GCG ATT CGC GCA TCG GCG ATC
MYG166:
GAT XCG XGA TGX GXG AAT XGX GAT XGA TGX GAT
(X: 5-methylcytosine)

### Substrate 2:

S-adenosyl-L-methionine

10× buffer:

0.5M Tris, pH 7.6, 50% Glycerol, 10 mM EDTA, 1 mg/ml BSA, 10 mM DTT

### DNMT1 Assay:

Protocol for screening at 15 μM and 45 μM inhibitor concentrations.
Assay format: 96-well

One μL of 450 μM Inhibitor: (15 μM final)or add 1 μL of 1.35 mM Inhibitor: (45 μM final) is added to the Enzyme Mix (0.03 μL of 25 uM of DNA Methyltransferase 1 to make 25 nM final, 0.2 μL of 390 uM of Hemi-methylated oligo to make 2.6 uM final, 1.65 μL of 10× Buffer [50 mM Tris-HCl pH7.6, 5% Glycerol, 1 mM EDTA, 100 ug/mL BSA, 1 mM DTT]. The mixture is pre-incubated at 37° C. for 10 min and then the SAM mix is added (2.25 uL of 40 uM of S-Adenosyl-L-Methionine to make 3 uM final, 1.65 uL of 10× Buffer [50 mM Tris-HCl pH7.6, 5% Glycerol, 1 mM EDTA, 100 ug/mL BSA, 1 mM DT). The final reaction is in 30 uL. The reaction is incubated at 37° C. for 15 min and then transferred onto DEAE filtermat (Wallac cat# 1450-522) using Tomtec cell harvester. The DEAE filtermat is washed with cold 20 mM NH4HCO3. When the filter is dry, it is covered with MeltiLex™ scintillant (Wallac cat#1450-441). Counting is done in a Wallac beta-counter.

DNMT3b2 assay follows identical protocol, at enzyme concentration of 188 nM.

The activities of a number of compounds according to the invention measured by various assays are displayed in the table below.

### Enzymatic DNMT1 and DNMT3b2 Data

| | DNMT1 IC50 | DNMT3b2 |
|---|---|---|
| | uM or % inh | IC50 uM or % |
| Ex. No. | @ 45 uM | inh @ 45 uM |
| 1 | 27 | 2 |
| 2 | 34 | 0.6 |
| 3 | 5.4 | 0.6 |
| 4 | >45 | 1.3 |
| 5 | 89 | 0.7 |
| 6 | 7.4 | 0.6 |
| 7 | 18 | 0.9 |
| 8 | >45 | 2 |
| 9 | 19 | 1 |
| 10 | >45 | 4.6 |
| 11 | >45 | 1 |
| 12 | >45 | 3 |
| 13 | >45 | 4 |
| 14 | >45 | 1 |
| 15 | >45 | 0.6 |
| 16 | 8 | 0.5 |
| 17 | >45 | 2 |
| 18 | 54% | 0.8 |
| 19 | 22 | 3.6 |
| 20 | >45 | 14 |
| 21 | 14 | 3.7 |
| 22 | 25 | 6 |
| 23 | >45 | 11 |
| 24 | 49% | >45 |
| 25 | >100 | 9 |
| 26 | 4 | 20 |
| 27 | 48% | >45 |
| 28 | 64% | 58% |
| 29 | 7 | 40 |
| 30 | 18 | 106 |
| 31 | 9 | >45 |
| 32 | 46% | >45 |
| 33 | 59% | 52% |
| 34 | 53% | >45 |
| 35 | 15 | 53% |
| 36 | 50% | >45 |
| 37 | 58% | >45 |
| 38 | 69% | 62% |
| 39 | 53% | >45 |
| 40 | 77% | 54% |
| 41 | 81% | >45 |
| 42 | 66% | 11 |
| 43 | 6.7 | 29 |
| 44 | 5 | 24 |
| 45 | 6 | 31 |
| 46 | 7 | 15 |
| 47 | 12 | 31 |
| 48 | 17 | 111 |
| 49 | 41 | 317 |
| 50 | 58% | >45 |
| 51 | 48% | 74% |
| 52 | 67% | >45 |
| 53 | 11 | >45 |
| 54 | 68% | >45 |
| 55 | 65% | >45 |
| 56 | 63% | >45 |
| 57 | 18 | >45 |
| 58 | 19.5 | >45 |
| 59 | 48% | >45 |
| 60 | 5.8 | >45 |
| 61 | 58% | >45 |
| 62 | 99 | 2.8 |
| 63 | 51% | 77% |
| 64 | 62% | 54% |
| 65 | 100 | 39 |
| 66 | 33 | 37 |
| 67 | 41 | 51 |
| 68 | 100 | 33 |
| 69 | 132 | 47 |
| 70 | >45 | 69% |
| 71 | >100 | 11 |
| 72 | >100 | 39 |
| 73 | 11.9 | 1.7 |
| 74 | 44 | >100 |
| 75 | 31 | 21 |
| 76 | 19 | 100 |
| 77 | 8 | 0.25 |
| 78 | 3 | 0.89 |
| 79 | 3.7 | 0.26 |
| 80 | 2.9 | 0.92 |
| 81 | 15 | 33 |
| 82 | 5 | 27 |
| 83 | 48 | 68 |
| 84 | 21 | 256 |
| 85 | 15 | >200 |
| 86 | 2.5 | 6.8 |
| 87 | 6 | >45 |
| 88 | 3 | 57 |
| 89 | 0.7 | 2 |
| 90 | 36 | >45 |
| 91 | 98 | 3 |
| 92 | 85 | 19 |
| 93 | 197 | 37 |
| 94 | 121 | 19 |
| 95 | 70 | 15 |
| 96 | 9 | 4 |
| 97 | 73 | 17 |
| 98 | 47 | 17 |
| 99 | 24 | 14 |
| 100 | 35 | 45 |
| 101 | >45 | 16 |
| 102 | >45 | 7 |
| 103 | 890 | 23 |

We claim:

1. A compound of formula (I): 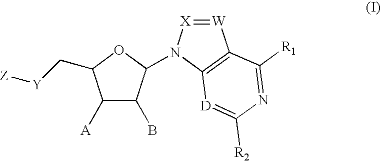
or a pharmaceutically acceptable salt or complex thereof, wherein
R₁ is H or NR₃R₄;
R₃ and R₄ independently represent H, C₁-C₆ alkyl, C₂-C₆ alkenyl, C₂-C₆ alkynyl, cycloalkyl, heterocyclyl, aryl, heteroaryl, —C₁-C₆-alkyl-cycloalkyl, —C₁-C₆-alkyl-heterocyclyl, —C₁-C₆-alkyl-aryl, —C₁-C₆-alkyl-heteroaryl, —C₁-C₆alkoxy-aryl or —(CH₂)₁₋₆-T, wherein C₁-C₆ alkyl, C₂-C₆ alkenyl, C₂-C₆ alkynyl, cycloalkyl, heterocyclyl, aryl and heteroaryl, at each occurrence, are optionally substituted; or
R₃ and R₄ taken together with the nitrogen to which they are attached form a C₅-C₉ heterocyclyl ring or a heteroaryl ring, wherein said ring is optionally substituted;
T is NH—C(═O)—R₁₄, —NH—SO₂—R₁₅, or —S—(CH₂)₁₋₃—R₁₄,
R₁₄ is C₁-C₆ alkyl, aryl or heteroaryl and R₁₅ is aryl, wherein C₁-C₆ alkyl, aryl, and heteroaryl, at each occurrence, are optionally substituted;
R₂ is H, halo, CF₃, C₁-C₆ alkyl, C₂-C₆ alkenyl, C₂-C₆ alkynyl, C₁-C₆ alkoxy, —NH—C₁-C₆ alkyl, or —S—C₁-C₆ alkyl, wherein C₁-C₆ alkyl, C₂-C₆ alkenyl, and C₂-C₆ alkynyl are each optionally substituted;
A and B independently are F, Cl, —OH, H, —NHR, or —OR;
R at each occurrence is independently benzyl or C₁-C₄ alkyl, wherein benzyl and C₁-C₄ alkyl are optionally substituted;
W is CH, N, CR, or C-halogen;
X is CH, N, C—C₁-C₆-alkyl, or C-halogen;
D is CH, or N;
Y is —S—, —O—, N(R₁₆)—, —CH═CH—, —S—CH₂—, —O—CH₂—,
where R₁₆ is H, C₁-C₆ alkyl, —C₁-C₆-alkyl-aryl, —C₁-C₆-alkyl-heteroaryl, or —C₂-C₆ alkenyl-aryl, wherein C₁-C₆ alkyl, aryl, and heteroaryl, at each occurrence, are optionally substituted;
Z is -L-C(H)(NH₂)—COOR₇, -L-NR₁₉R₂₀, or heterocyclyl, wherein heterocyclyl is optionally substituted;
L is a bond or is —(CR₁₇R₁₈)₁₋₆—;
each R₁₇ and R₁₈ independently is H or C₁-C₆-alkyl, wherein C₁-C₆-alkyl is optionally substituted;
R₁₉ and R₂₀ independently are H, C₁-C₆-alkyl, heteroaryl, or H₂N—C(═NH)—CH₂—, wherein
C₁-C₆-alkyl and heteroaryl are optionally substituted; and
R₇ is H or C₁-C₆-alkyl,
wherein compounds selected from the group consisting of: 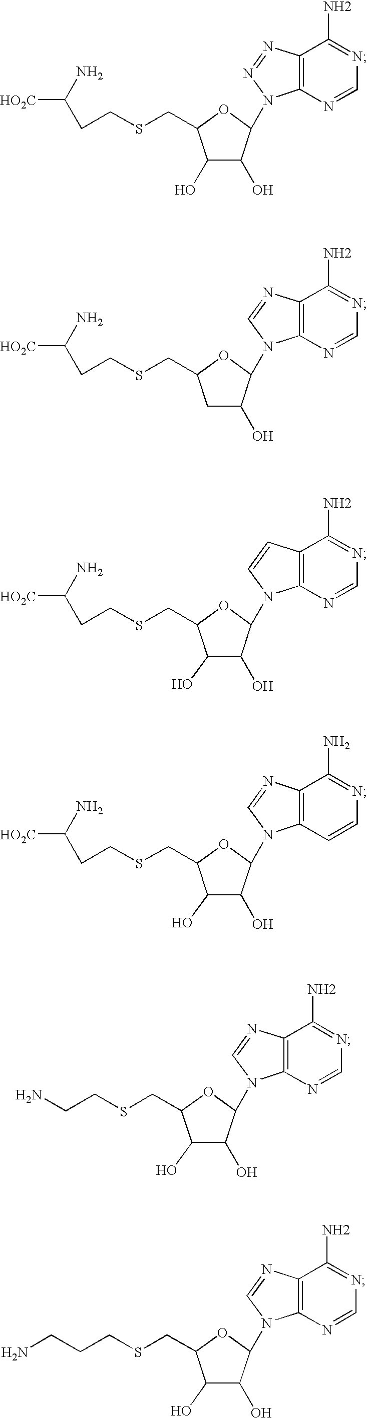 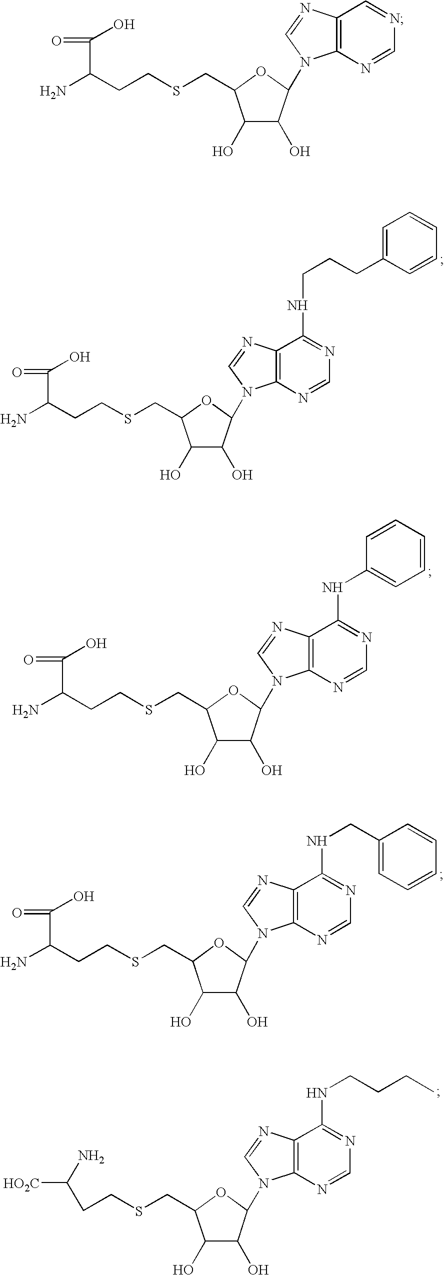 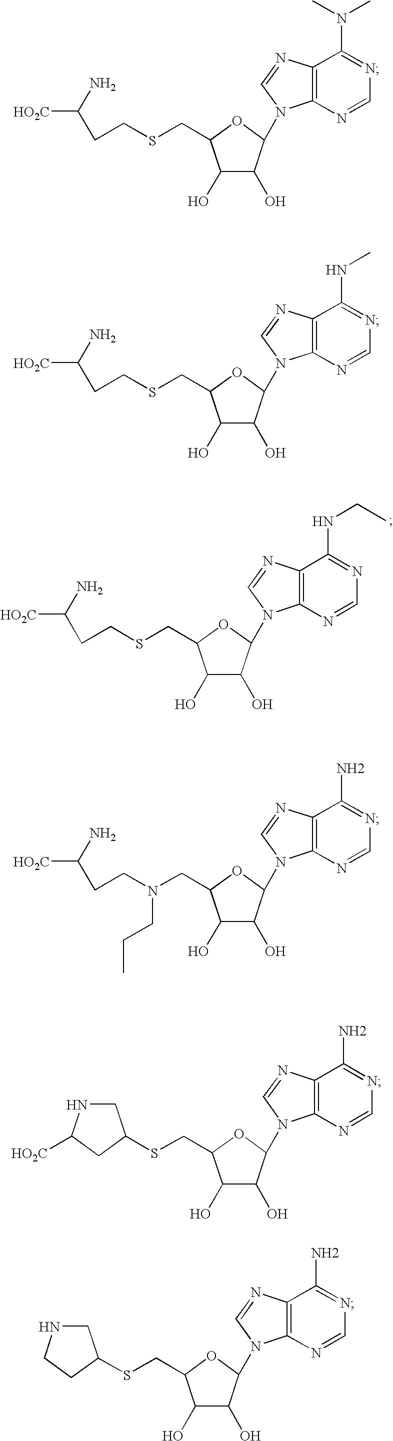 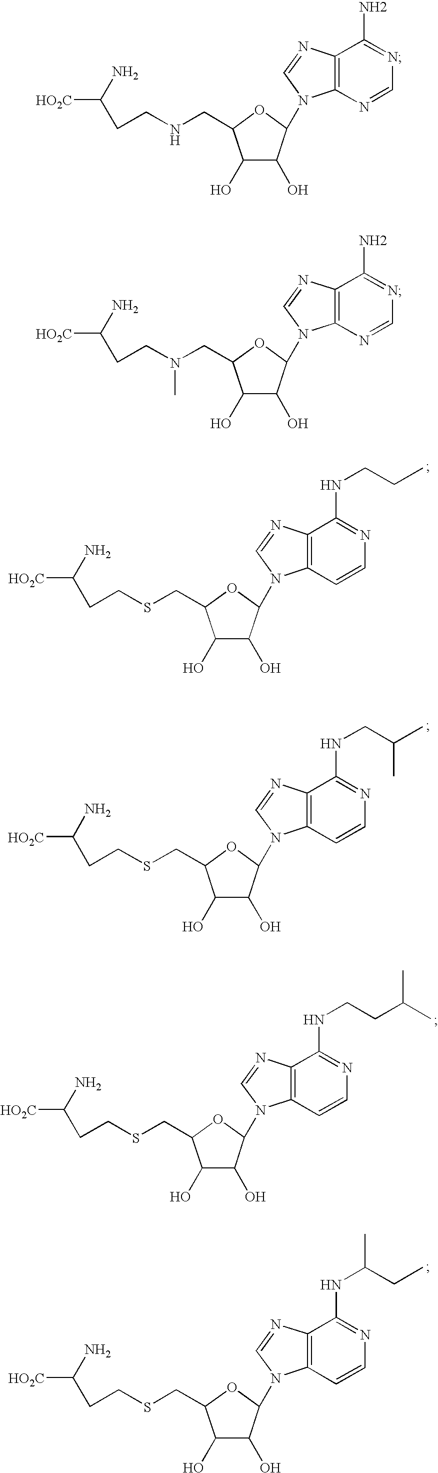 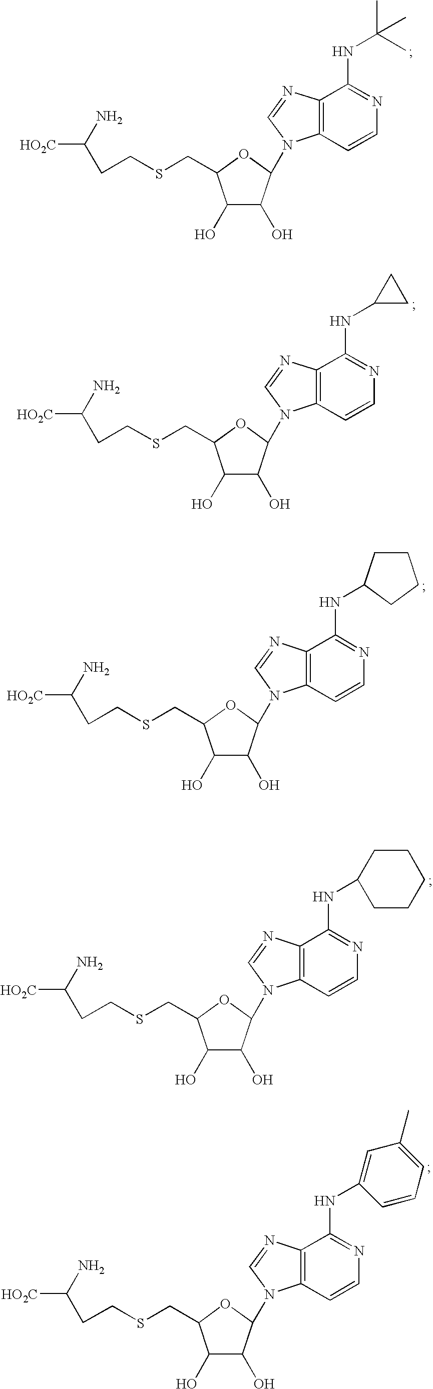 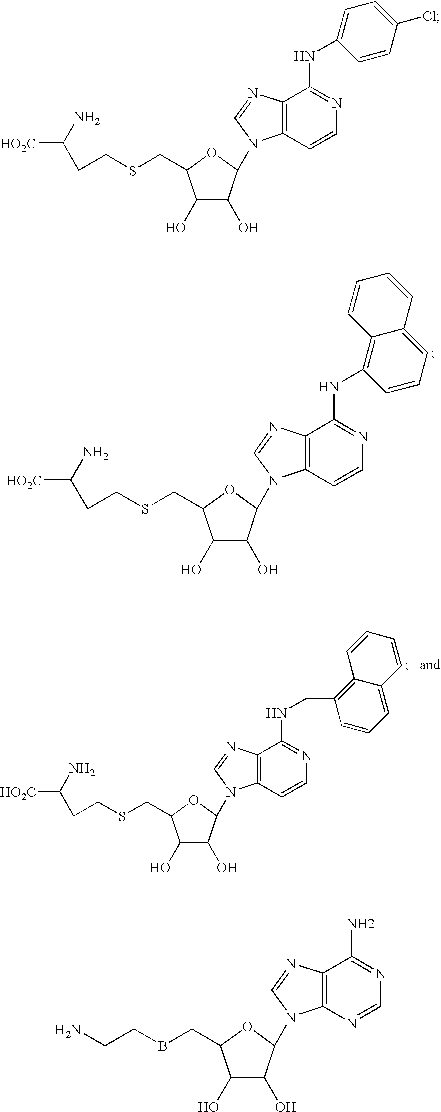
are excluded.

2. A compound according to claim 1, represented by formula (II) 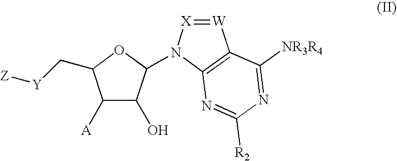
or a pharmaceutically acceptable salt or complex thereof, wherein
A is H, halogen, or OH;
R₂ is H, halo, C₁-C₆ alkyl, C₂-C₆ alkenyl or —S—C₁-C₆ alkyl, wherein C₁-C₆ alkyl and C₂-C₆ alkenyl, at each occurrence, are optionally substituted;
R₃ and R₄ independently represent H, C₁-C₆ alkyl, C₂-C₆ alkenyl, cycloalkyl, heterocyclyl, aryl, heteroaryl, —C₁-C₆-alkyl-cycloalkyl, —C₁-C₆-alkyl-heterocyclyl, —C₁-C₆-alkyl-aryl, —C₁-C₆-alkyl-heteroaryl, —C₁-C₆alkoxy-aryl or —(CH₂)₁₋₆-T, wherein C₁-C₆ alkyl, C₂-C₆ alkenyl, cycloalkyl, heterocyclyl, aryl and heteroaryl, at each occurrence, are optionally substituted; or
R₃ and R₄ taken together with the nitrogen to which they are attached form a C₅-C₉ heterocyclyl ring or a heteroaryl ring, wherein said ring is optionally substituted;
T is NH—C(═O)—R₁₄, —NH—SO₂—R₁₅, or —S—(CH₂)₁₋₃—R₁₄,
R₁₄ is C₁-C₆ alkyl, aryl or heteroaryl and R₁₅ is aryl, wherein C₁-C₆ alkyl, aryl, and heteroaryl, at each occurrence, are optionally substituted;
W and X are independently CH or N;
Y is S, O, or N(R₁₆)—,
R₁₆ is H, C₁-C₆ alkyl, —C₁-C₆-alkyl-aryl, —C₁-C₆-alkyl-heteroaryl, or —C₂-C₆ alkenyl-aryl, wherein C₁-C₈ alkyl, aryl, and heteroaryl, at each occurrence, are optionally substituted;
Z is -L-C(H)(NH₂)—COOR₇, -L-NR₁₉R₂₀, or heterocyclyl, wherein heterocyclyl is optionally substituted;
L is a bond or is —(CR₁₇R₁₈)₁₋₆—;
R₁₇ and R₁₈ independently are H or C₁-C₆-alkyl, wherein C₁-C₆-alkyl is optionally substituted; and
R₁₉ and R₂₀ independently are H, C₁-C₆-alkyl, heteroaryl, or H₂N—C(═NH)CH₂—, wherein
C₁-C₆-alkyl and heteroaryl are optionally substituted
R₇ is H or C₁-C₆-alkyl.

3. A compound according to claim 2, of the formula II-A: 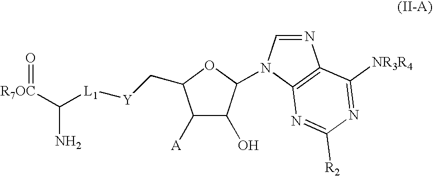
or a pharmaceutically acceptable salt or complex thereof, wherein
L₁ is —(CR₁₇R₁₈)₁₋₆—; and
R₁₇ and R₁₈ independently are H or C₁-C₆-alkyl, wherein C₁-C₆-alkyl is optionally substituted.

4. A compound according to claim 3, wherein A is OH.

5. A compound according to claim 3, wherein R₇ is H.

6. A compound according to claim 3, wherein L₁ is —CH₂CH₂—.

7. A compound according to claim 3, wherein R₂ is H, halogen, C₁-C₃ alkyl, —S—C₁-C₂ alkyl, or C₂-C₃ alkenyl.

8. A compound according to claim 3, wherein Y is S.

9. A compound according to claim 3, wherein R₃ and R₄ are independently H, C₁-C₆ alkyl, C₂-C₆ alkenyl, cycloalkyl, heterocyclyl, aryl, heteroaryl, —C₁-C₆-alkyl-cycloalkyl, —C₁-C₆-alkyl-aryl, —C₁-C₆-alkyl-heteroaryl, —C₁-C₆alkoxy-aryl or —(CH₂)₁₋₆-T, wherein C₁-C₆ alkyl, C₂-C₆ alkenyl, cycloalkyl, heterocyclyl, aryl and heteroaryl, at each occurrence, are optionally substituted, or R₃ and R₄ taken together with the nitrogen to which they are attached form a C₅-C₉ heterocyclyl ring or a heteroaryl ring, wherein said ring is optionally substituted.

10. A compound according to claim 2, of formula II-B: 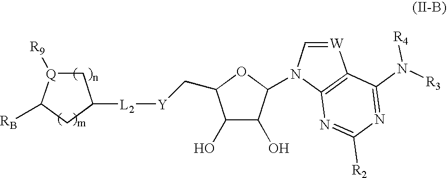
or a pharmaceutically acceptable salt or complex thereof, wherein
m is 0 or 1;
n is 1 or 2;
L₂ is a bond or is —CH₂—;
R₂ is H or halogen;
R₃ is H, C₁-C₆ alkyl, or —C₁-C₆-alkyl-aryl, wherein C₁-C₆ alkyl and aryl, at each occurrence, are optionally substituted;
R₄ is H or C₁-C₆ alkyl, wherein C₁-C₆ alkyl is optionally substituted;
R₈ is H, —CO₂H, or CO₂CH₃;
R₉ is absent, H or C₁-C₆ alkyl, wherein C₁-C₆ alkyl is optionally substituted;
W is N or CH;
Y is S or O; and
Q is N, CH or O, provided that when Q is O, R₉ is absent.

11. A compound according to claim 10 wherein R₃ and R₄ are both H.

12. A compound according to claim 10 wherein R₃ is —C₁-C₆-alkyl-aryl and R₄ is H, wherein aryl is optionally substituted.

13. A compound according to claim 2, of the formula II-C: 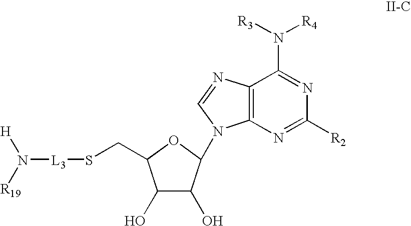
or a pharmaceutically acceptable salt or complex thereof, wherein
R₂ is H or halo;
R₃ and R₄ independently represent H, C₁-C₆ alkyl, C₂-C₆ alkenyl, C₂-C₆ alkynyl, cycloalkyl, aryl or (—C₁-C₆-alkyl)-aryl, wherein C₁-C₆ alkyl, C₂-C₆ alkenyl, C₂-C₆ alkynyl, cycloalkyl, aryl and (—C₁-C₆-alkyl)-aryl are each optionally substituted; or
R₃ and R₄ taken together with the nitrogen to which they are attached form a C₅-C₉-heterocyclyl ring or a heteroaryl ring, wherein said ring is optionally substituted;
L₃ is a bond or is —(CR₁₇R₁₈)₁₋₆—;
R₁₇ and R₁₈ at each occurrence are independently H or C₁-C₆-alkyl, wherein C₁-C₆-alkyl is optionally substituted;
R₁₉ is H, C₁-C₆-alkyl, heteroaryl, or H₂N—C(═NH)—CH₂—, wherein C₁-C₆-alkyl and heteroaryl are optionally substituted.

14. A compound according to claim 13 wherein L₃ is —CHR₁₇CHR₁₈—, R₁₇ and R₁₈ independently are H, or C₁-C₆ alkyl, and C₁-C₆ alkyl at each occurrence is optionally substituted.

15. A compound according to claim 14 wherein R₁₇ and R₁₈ independently are H, or C₁-C₆ alkyl, and C₁-C₆ alkyl is unsubstituted or is substituted with NH₂.

16. A compound according to claim 13 wherein L₃ is —CH₂CH₂CH₂—.

17. A method of inhibiting DNMT1 and/or DNMT3b2 enzymes in a cell, comprising contacting a cell in which inhibition of DNMT1 or DNMT3b2 is desired with a compound according to claim 1.

18. A composition comprising a compound according to claim 1, or salt or complex thereof, together with a pharmaceutically acceptable excipient, diluent, or carrier.


SEQUENCE LISTING (2 sequences)

SEQ ID NO: 1 - protein, 1616 aa, Homo sapiens
MPARTAPARVPTLAVPAISLPDDVRRRLKDLERDSLTEKECVKEKLNLLHEFLQTEIKNQLCDLETKLRKEELSEEGYLAKVKSLLNKDLSLENGAHAYNREVNGRLENGNQARSEARRVGMADANSPPKPLSKPRTPRRSKSDGEAKPEPSPSPRITRKSTRQTTITSHFAKGPAKRKPQEESERAKSDESIKEEDKDQDEKRRRVTSRERVARPLPAEEPERAKSGTRTEKEEERDEKEEKRLRSQTKEPTPKQKLKEEPDREARAGVQADEDEDGDEKDEKKHRSQPKDLAAKRRPEEKEPEKVNPQISDEKDEDEKEEKRRKTTPKEPTEKKMARAKTVMNSKTHPPKCIQCGQYLDDPDLKYGQHPPDAVDEPQMLTNEKLSIFDANESGFESYEALPQHKLTCFSVYCKHGHLCPIDTGLIEKNIELFFSGSAKPIYDDDPSLEGGVNGKNLGPINEWWITGFDGGEKALIGFSTSFAEYILMDPSPEYAPIFGLMQEKIYISKIVVEFLQSNSDSTYEDLINKIETTVPPSGLNLNRFTEDSLLRHAQFVVEQVESYDEAGDSDEQPIFLTPCMRDLIKLAGVTLGQRRAQARRQTIRHSTREKDRGPTKATTTKLVYQIFDTFFAEQIEKDDREDKENAFKRRRCGVCEVCQQPECGKCKACKDMVKFGGSGRSKQACQERRCPNMAMKEADDDEEVDDNIPEMPSPKKMHQGKKKKQNKNRISWVGEAVKTDGKKSYYKKVCIDAETLEVGDCVSVIPDDSSKPLYLARVTALWEDSSNGQMFHAHWFCAGTDTVLGATSDPLELFLVDECEDMQLSYIHSKVKVIYKAPSENWAMEGGMDPESLLEGDDGKTYFYQLWYDQDYARFESPPKTQPTEDNKFKFCVSCARLAEMRQKEIPRVLEQLEDLDSRVLYYSATKNGILYRVGDGVYLPPEAFTFNIKLSSPVKRPRKEPVDEDLYPEHYRKYSDYIKGSNLDAPEPYRIGRIKEIFCPKKSNGRPNETDIKIRVNKFYRPENTHKSTPASYHADINLLYWSDEEAVVDFKAVQGRCTVEYGEDLPECVQVYSMGGPNRFYFLEAYNAKSKSFEDPPNHARSPGNKGKGKGKGKGKPKSQACEPSEPEIEIKLPKLRTLDVFSGCGGLSEGFHQAGISDTLWAIEMWDPAAQAFRLNNPGSTVFTEDCNILLKLVMAGETTNSRGQRLPQKGDVEMLCGGPPCQGFSGMNRFNSRTYSKFKNSLVVSFLSYCDYYRPRFFLLENVRNFVSFKRSMVLKLTLRCLVRMGYQCTFGVLQAGQYGVAQTRRRAIILAAAPGEKLPLFPEPLHVFAPRACQLSVVVDDKKFVSNITRLSSGPFRTITVRDTMSDLPEVRNGASALEISYNGEPQSWFQRQLRGAQYQPILRDHICKDMSALVAARMRHIPLAPGSDWRDLPNIEVRLSDGTMARKLRYTHHDRKNGRSSSGALRGVCSCVEAGKACDPAARQFNTLIPWCLPHTGNRHNHWAGLYGRLEWDGFFSTTVTNPEPMGKQGRVLHPEQHRVVSVRECARSQGFPDTYRLFGNILDKHRQVGNAVPPPLAKAIGLEIKLCMLAKARESASAKIKEEEAAKD

SEQ ID NO: 2 - protein, 833 aa, Homo sapiens
MKGDTRHLNGEEDAGGREDSILVNGACSDQSSDSPPILEAIRTPEIRGRRSSSRLSKREVSSLLSYTQDLTGDGDGEDGDGSDTPVMPKLFRETRTRSESPAVRTRNNNSVSSRERHRPSPRSTRGRQGRNHVDESPVEFPATRSLRRRATASAGTPWPSPPSSYLTIDLTDDTEDTHGTPQSSSTPYARLAQDSQQGGMESPQVEADSGDGDSSEYQDGKEFGIGDLVWGKIKGFSWWPAMVVSWKATSKRQAMSGMRWVQWFGDGKFSEVSADKLVALGLFSQHFNLATFNKLVSYRKAMYHALEKARVRAGKTFPSSPGDSLEDQLKPMLEWAHGGFKPTGIEGLKPNNTQPENKTRRRTADDSATSDYCPAPKRLKTNCYNNGKDRGDEDQSREQMASDVANNKSSLEDGCLSCGRKNPVSFHPLFEGGLCQTCRDRFLELFYMYDDDGYQSYCTVCCEGRELLLCSNTSCCRCFCVECLEVLVGTGTAAEAKLQEPWSCYMCLPQRCHGVLRRRKDWNVRLQAFFTSDTGLEYEAPKLYPAIPAARRRPIRVLSLFDGIATGYLVLKELGIKVGKYVASEVCEESIAVGTVKHEGNIKYVNDVRNITKKNIEEWGPFDLVIGGSPCNDLSNVNPARKGLYEGTGRLFFEFYHLLNYSRPKEGDDRPFFWMFENVVAMKVGDKRDISRFLECNPVMIDAIKVSAAHRARYFWGNLPGMNRPVIASKNDKLELQDCLEYNRIAKLKKVQTITTKSNSIKQGKNQLFPVVMNGKEDVLWCTELERIFGFPVHYTDVSNMGRGARQKLLGRSWSVPVIRHLFAPLKDYFACE